(19) United States
(12) Patent Application Publication
(10) Pub. No.: US 2022/0243249 A1
(43) Pub. Date: Aug. 4, 2022

(54) ANTAGONISTS OF CAMKII-DELTA 9 AND USES THEREOF

(71) Applicant: PEKING UNIVERSITY, Beijing (CN)

(72) Inventors: Mao ZHANG, Beijing (CN); Yan ZHANG, Beijing (CN); Rui-Ping XIAO, Beijing (CN); Hua GAO, Beijing (CN)

(73) Assignee: PEKING UNIVERSITY, Beijing (CN)

(21) Appl. No.: 17/619,232

(22) PCT Filed: Jun. 11, 2020
(86) PCT No.: PCT/CN2020/095555

§ 371 Date: Dec. 14, 2021

(30) Foreign Application Priority Data
Jun. 18, 2019 (CN) PCT/CN2019/091685
Aug. 30, 2019 (CN) PCT/CN2019/103678

(51) Int. Cl. C12Q 1/48

(52) U.S. Cl. CPC C12Q 1/485; G01N 2333/075; G01N 2800/32; G01N 2500/02

(57) ABSTRACT

Provided are methods of treating or preventing Ca²⁺/calmodulin-dependent kinase II (CaMKII)-mediated diseases, methods of alleviating cardiac injury, methods of stimulating the activity of ubiquitin-conjugating enzyme, methods of preventing degradation of ubiquitin-conjugating enzyme, methods of preventing cardiomyocyte death, methods of reducing DNA damage in a cell, methods for diagnosing CaMKII-mediated diseases, kits for diagnosing CaMKII-mediated diseases, biomarkers for diagnosing a CaMKII-mediated disease, and use of CaMKIIδ9 as a biomarker for diagnosing a CaMKII-mediated disease. Also provided herein are methods for identifying molecules, isolated polypeptides, isolated nucleic acids, and antagonists thereof.

### TECHNICAL FIELD

The present invention relates to the biomedical field. In particular, the present invention relates to antagonists of CaMKII-δ9 and uses thereof.

### BACKGROUND ART

Throughout the lifespan of an organism, the genome is constantly attacked by various internal and external stress signals, resulting in DNA damage. Excessive DNA damage impairs genomic integrity and blocks DNA replication and transcription (Campisi, J. & d′Adda di Fagagna, F. Nature reviews. Molecular cell biology 8, 729-740, doi:10. 1038/nrm2233 (2007)). As a safeguard, DNA repair defends against harmful DNA damage, thereby preserving the genome stability and cell viability. Aberrant DNA repair causes the accumulation of DNA damage and genome instability, resulting in cell death. Since mammalian cardiomyocytes have little or no capacity for regeneration, loss of terminally-differentiated cardiomyocytes is a common etiology of many types of heart diseases, including myocardial infarction, cardiomyopathy and heart failure. However, the mechanism underlying cardiomyocyte DNA repair remains largely unknown.

Ca²⁺/calmodulin-dependent kinase II (CaMKII) is a family of multifunctional serine/threonine protein kinases, which is involved in the regulation of cardiac cell survival and cell death (Erickson, J. R., He, B. J., Grumbach, I. M. & Anderson, M. E. Physiological reviews 91, 889-915, doi:10.1152/physrev.00018.2010 (2011)). CaMKII is encoded by four genes, CaMKII-α, β, γ, and δ, and CaMKII-δ is predominantly expressed in the heart. CaMKII-δ is alternatively spliced at two variable domains—between exons 13 and 17 (Variable domain 1), and exons 20 and 22 (Variable domain 2)—generating 11 different splice variants. In particular, CaMKII-δ2 (also named CaMKII-δC) and CaMKII-δ3 (or CaMKII-δB) have been previously identified as the major cardiac splice variants, which are located in the cytoplasm and the nucleus, respectively. Emerging evidence suggests that CaMKII-δ2 and δ3 elicit different even opposing effects on cardiac myocyte viability (Peng, W. et al. Circulation research 106, 102-110, doi:10.1161/CIRCRESAHA.109.210914 (2010)). However, little is known about the physiological and pathological functions of CaMKII-δ9 in the heart.

### SUMMARY OF THE INVENTION

In the present invention, the inventors have identified CaMKII-δ9, rather than the well-studied CaMKII-δ2 and δ3, as the predominant CaMKII-δ splice variant in human heart. In response to various stimuli, CaMKII-δ9 is upregulated, and much more potent in triggering cardiomyocyte DNA damage, genome instability, and cardiac pathology than other splice variants (CaMKII-δ2 and δ3). The inventors also have deciphered that the peptide encoded by exons 13-16-17, the feature sequence of CaMKII-δ9, confers the splice variant-specific regulation of UBE2T phosphorylation and degradation.

In one aspect, the present invention discloses methods of treating or preventing a CaMKII-mediated disease in a subject, comprising administering to the subject an effective amount of an antagonist of CaMKII-δ9.

In another aspect, the present invention discloses methods of alleviating cardiac injury in a subject, comprising administering to the subject an effective amount of an antagonist of CaMKII-δ9.

In yet another aspect, the present invention discloses methods of stimulating the level or activity of ubiquitin-conjugating enzyme in a subject, comprising administering to the subject an effective amount of an antagonist of CaMKII-δ9.

In yet another aspect, the present invention discloses methods of preventing degradation of ubiquitin-conjugating enzyme in a subject, comprising administering to the subject an effective amount of an antagonist of CaMKII-δ9.

In yet another aspect, the present invention discloses methods of preventing cardiomyocyte death in a sample, comprising contacting the sample with an effective amount of an antagonist of CaMKII-δ9.

In yet another aspect, the present invention discloses methods of reducing DNA damage in a cell, comprising contacting the cell with an effective amount of an antagonist of CaMKII-δ9.

In some embodiments, the antagonist of CaMKII-δ9 disclosed herein is capable of inhibiting the activation of CaMKII-δ9 or inhibiting the kinase activity of CaMKII-δ9. In some embodiments, CaMKII-δ9 is activated by the phosphorylation and/or oxidation of CaMKII-δ9 per se. In some embodiments, the kinase activity of CaMKII-δ9 is shown as its capability of phosphorylating its substrate, for example, ubiquitin-conjugating enzyme, or more specifically ubiquitin-conjugating enzyme 2T (UBE2T). In some embodiments, the antagonist of the present invention is an antagonist for inhibiting the phosphorylation of ubiquitin-conjugating enzyme. In some embodiments, the ubiquitin-conjugating enzyme is UBE2T. In some embodiment, the antagonist is an antagonist for inhibiting the phosphorylation of UBE2T at Ser110. In some embodiments, the antagonist is a specific antagonist of CaMKII-δ9. In some embodiments, the antagonist inhibits the level or activity of CaMKII-δ9 but does not significantly inhibit the level or activity of CaMKII-δ2 or CaMKII-δ3.

In some embodiments, the antagonist is an antibody that specifically recognizes CaMKII-δ9, a small molecule compound that binds to CaMKII-δ9, an RNAi molecule that targets an encoding sequence of CaMKII-δ9, an antisense nucleotide that targets an encoding sequence of CaMKII-δ9, or an agent that competes with CaMKII-δ9 to bind to its substrate.

In some embodiments, the antibody is a monoclonal antibody or a polyclonal antibody. In some embodiments, the antibody is a humanized antibody, a chimeric antibody or a fully human antibody. In some embodiments, the antibody binds to the amino acid sequence encoded by exon 16 of CaMKII-δ gene.

In some embodiments, the RNAi molecule is a small interfering RNA (siRNA), a small hairpin RNA (shRNA) or a microRNA (miRNA). In some embodiments, the RNAi molecule has 10-100 bases. In some embodiments, the antisense nucleotide is modified to improve its stability. In some embodiments, the RNAi molecule and the antisense nucleotide bind to exon 16 of CaMKII-δ gene. In some embodiments, the RNAi molecule and the antisense nucleotide binds to exon 13 and exon 16 (also referred as “exons 13-16” in the present invention) of CaMKII-δ gene, or exon 16 and exon 17 (also referred as “exons 16-17” in the present invention) of CaMKII-δ gene, or exon 13 and exon 16 and exon 17 (also referred as “exons 13-16-17” in the present invention) of CaMKII-δ gene.

In some embodiments, the agent that competes with CaMKII-δ9 to bind to its substrate is a vector that expresses CaMKII-δ9 which is without phosphorylation or oxidation function. In some embodiments, the vector is an adeno-associated virus (AAV), an adenovirus, a lentivirus, a retrovirus, or a plasmid. In some embodiments, the AAV is AAV1, AAV2, AAV5, AAV8, AAV9 or AAVrh10.

In some embodiments, the subject is a human or non-human primate. In some embodiments, the non-human primate is a rhesus monkey. In some embodiments, the subject is a rodent, for example, rat or mouse.

In some embodiments, the CaMKII-mediated disease is associated with an increased level or activity of CaMKII-δ9. In some embodiments, the CaMKII-mediated disease is a heart disease or a metabolic disease. In some embodiments, the heart disease is selected from the group consisting of cardiomyopathy, myocarditis, diabetic heart disease, myocardial ischemia, cardiac ischemia/reperfusion injury, myocardial infarction, heart failure, arrhythmia, heart rupture, angina, cardiac hypertrophy, cardiac injury, hypertensive heart disease, rheumatic heart disease, angina, myocarditis, coronary heart disease and pericarditis. In some embodiments, the heart disease is hypertrophic cardiomyopathy. In some embodiments, the metabolic disease is selected from the group consisting of insulin resistance, obesity, diabetes, hypertension, dyslipidemia, diabetic cerebrovascular diseases, diabetic ocular complications, diabetic neuropathy, diabetic foot, hyperinsulinemia, hypercholesterolemia, hyperglycaemia, hyperlipemia, gout and hyperuricemia.

In another aspect, the present invention relates to methods for diagnosing a CaMKII-mediated disease in a subject comprising: (a) obtaining a test biological sample of the subject; (b) detecting a level or activity of CaMKII-δ9 in the test biological sample; wherein the level or activity of CaMKII-δ9 detected in the test biological sample of the subject is indicative of the subject developing or with an increased probability of developing a CaMKII-mediated disease.

In some embodiments, the level or activity of CaMKII-δ9 in the test biological sample is detected by contacting the sample with a reagent that specifically binds to CaMKII-δ9. In some embodiments, the level or activity of CaMKII-δ9 detected in the test biological sample is compared to a reference level or activity of CaMKII-δ9 detected in a reference sample. In some embodiments, a higher level or activity of the CaMKII-δ9 detected in the test biological sample than the reference level or activity of CaMKII-δ9 is indicative of the subject developing or with an increased probability of developing a CaMKII-mediated disease. In some embodiments, the reference sample is from a healthy subject or is a sample obtained from the same subject earlier or later than the test biological sample. In some embodiments, the test biological sample is from the heart of the subject. In some embodiments, the subject is a human or non-human primate.

In another aspect, the present invention discloses kits for diagnosing a CaMKII-mediated disease in a subject, comprising an antibody or an antibody fragment that specifically recognizes CaMKII-δ9.

In another aspect, the present invention discloses a biomarker for diagnosing a CaMKII-mediated disease in a subject, wherein the biomarker includes the full length protein sequence of CaMKII-δ9 or a fragment thereof. In some embodiments, the biomarker includes the amino acid sequence set forth in SEQ ID NOs: 1-5.

In another aspect, the present invention discloses use of CaMKII-δ9 as a biomarker for diagnosing a CaMKII-mediated disease in a subject.

In another aspect, the present invention discloses methods for identifying a molecule that inhibits the activity of CaMKII-δ9, comprising contacting the molecule with CaMKII-δ9 and UBE2T, and determining whether the phosphorylation of UBE2T is inhibited, wherein the inhibition of the phosphorylation of UBE2T identifies a molecule that inhibits CaMKII-δ9.

In yet another aspect, the present invention discloses methods for identifying a molecule that inhibits the phosphorylation capability of CaMKII-δ9, comprising contacting the molecule with CaMKII-δ9 and UBE2T, and determining whether the phosphorylation of UBE2T is inhibited, wherein the inhibition of the phosphorylation of UBE2T identifies a molecule that inhibits the phosphorylation capability of CaMKII-δ9.

In yet another aspect, the present invention discloses methods for identifying a molecule that inhibits the phosphorylation and/or oxidation of CaMKII-δ9 per se, comprising contacting the molecule with CaMKII-δ9 and an antibody that can detect the phosphorylation and/or oxidation status of CaMKII-δ9, and determining whether the level of phosphorylated and/or oxidized CaMKII-δ9 is decreased, wherein a decreased level of phosphorylated and/or oxidized CaMKII-δ9 identifies a molecule that inhibits the phosphorylation and/or oxidation of CaMKII-δ9.

In yet another aspect, the present invention discloses methods for identifying a molecule that treats or prevents a CaMKII-mediated disease, comprising contacting the molecule with CaMKII-δ9 and UBE2T, and determining whether the phosphorylation of UBE2T is inhibited, wherein the inhibition of the phosphorylation of UBE2T identifies a molecule that treats or prevents a CaMKII-mediated disease.

In yet another aspect, the present invention discloses methods for identifying a molecule that treats or prevents a CaMKII-mediated disease, comprising contacting the molecule with CaMKII-δ9 and an antibody that can detect the phosphorylation and/or oxidation status of CaMKII-δ9, and determining whether the level of phosphorylated and/or oxidized CaMKII-δ9 is decreased, wherein a decreased level of phosphorylated and/or oxidized CaMKII-δ9 identifies a molecule that treats or prevents a CaMKII-mediated disease.

In yet another aspect, the present invention discloses methods for identifying a molecule that alleviates cardiac injury, comprising contacting the molecule with CaMKII-δ9 and UBE2T, and determining whether the phosphorylation of UBE2T is inhibited, wherein the inhibition of the phosphorylation of UBE2T identifies a molecule that alleviates cardiac injury.

In yet another aspect, the present invention discloses methods for identifying a molecule that alleviates cardiac injury, comprising contacting the molecule with CaMKII-δ9 and an antibody that can detect the phosphorylation and/or oxidation status of CaMKII-δ9, and determining whether the level of phosphorylated and/or oxidized CaMKII-δ9 is decreased, wherein a decreased level of phosphorylated and/or oxidized CaMKII-δ9 identifies a molecule that alleviates cardiac injury.

In yet another aspect, the present invention discloses methods for identifying a molecule that prevents cardiomyocyte death, comprising contacting the molecule with CaMKII-δ9 and UBE2T, and determining whether the phosphorylation of UBE2T is inhibited, wherein the inhibition of the phosphorylation of UBE2T identifies a molecule that prevents cardiomyocyte death.

In yet another aspect, the present invention discloses methods for identifying a molecule that prevents cardiomyocyte death, comprising contacting the molecule with CaMKII-δ9 and an antibody that can detect the phosphorylation and/or oxidation status of CaMKII-δ9, and determining whether the level of phosphorylated and/or oxidized CaMKII-δ9 is decreased, wherein a decreased level of phosphorylated and/or oxidized CaMKII-δ9 identifies a molecule that prevents cardiomyocyte death.

In yet another aspect, the present invention discloses methods for identifying a molecule that reduces DNA damage, comprising contacting the molecule with CaMKII-δ9 and UBE2T, and determining whether the phosphorylation of UBE2T is inhibited, wherein the inhibition of the phosphorylation of UBE2T identifies a molecule that reduces DNA damage.

In some embodiments, the phosphorylation of UBE2T is at Ser110.

In yet another aspect, the present invention discloses methods for identifying a molecule that reduces DNA damage, comprising contacting the molecule with CaMKII-δ9 and an antibody can detect the phosphorylation and/or oxidation status of CaMKII-δ9, and determining whether the level of phosphorylated and/or oxidized CaMKII-δ9 is decreased, wherein a decreased level of phosphorylated and/or oxidized CaMKII-δ9 identifies a molecule that reduces DNA damage.

In another aspect, the present invention discloses isolated CaMKII-δ polypeptides comprising an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5.

In yet another aspect, the present invention discloses isolated CaMKII-5 nucleic acids comprising a nucleic acid sequence encoding the polypeptide of the present invention. In some embodiments, the CaMKII-δ nucleic acid comprises one of the nucleic acid sequences selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and a nucleic acid sequence having at least 80% homology to SEQ ID NOs: 6-19.

In yet another aspect, the present invention discloses CaMKII antagonists capable of inhibiting the level or activity of CaMKII-δ9. In some embodiments, the antagonist is an antagonist for inhibiting the phosphorylation of an ubiquitin-conjugating enzyme. In some embodiments, the ubiquitin-conjugating enzyme is UBE2T. In some embodiments, the antagonist is an antagonist for inhibiting the phosphorylation of UBE2T at Ser110. In some embodiments, the antagonist is a specific antagonist of CaMKII-δ9. In some embodiments, the antagonist inhibits the level or activity of CaMKII-δ9 but does not significantly inhibit the level or activity of CaMKII-δ2 or CaMKII-δ3. In some embodiments, the antagonist is an antibody that binds to the amino acid sequence encoded by exon 16 of CaMKII-δ gene, an RNAi molecule that targets exon 16 of CaMKII-δ gene, or an antisense nucleotide that targets exon 16 of CaMKII-δ gene. In some embodiments, the antagonist is an antibody that binds to the amino acid sequence encoded by exon 13 and exon 16 of CaMKII-δ gene, an RNAi molecule that targets exon 13 and exon 16 of CaMKII-5 gene, or an antisense nucleotide that targets exon 13 and exon 16 of CaMKII-δ gene. In some embodiments, the antagonist is an antibody that binds to the amino acid sequence encoded by exon 16 and exon 17 of CaMKII-δ gene, an RNAi molecule that targets exon 16 and exon 17 of CaMKII-δ gene, or an antisense nucleotide that targets exon 16 and exon 17 of CaMKII-5 gene. In some embodiments, the antagonist is an antibody that binds to the amino acid sequence encoded by exon 13, exon 16 and exon 17 of CaMKII-δ gene, an RNAi molecule that targets exon 13, exon 16 and exon 17 of CaMKII-δ gene, or an antisense nucleotide that targets exon 13, exon 16 and exon 17 of CaMKII-δ gene. In some embodiments, the antagonist is an antibody that binds to the amino acid sequence of the full-length CaMKII-δ9, an RNAi molecule that targets the encoding sequence of the full-length CaMKII-δ9, or an antisense nucleotide that targets the encoding sequence of the full-length CaMKII-δ9.

In yet another aspect, the present invention discloses a pharmaceutical composition comprising the antagonist of the present invention and a pharmaceutically acceptable carrier.

### BRIEF DESCRIPTION OF THE FIGURES

FIG. 1 illustrates that CaMKII-δ9 is an important cardiac cytosolic CaMKII-δ splice variant. (a), Splicing landscape and expression levels of all CaMKII-δ splice variants in the heart of mouse, rat, rhesus monkey, and human. The transcript map visualizes the 11 reported alternatively-spliced variants (rows) obtained by SMRT sequencing. Exons (columns) are in black if present and are numbered at bottom (lower columns, UTR regions; black lines, exon linkage). The length of each box is proportional to the length of each exon, and the percentage of each splicing variant is shown in bar graphs (right panels, CaMKII-δ9 in gray), and the absolute read numbers of some of the major variants are shown on the bars (3 samples were pooled together in each species). (b), Reciprocal immunoprecipitation of total protein from mouse heart with exon 21 antibody and probed with exon 16 antibody, and vice versa. Input lanes are from longer exposure of the same membrane. n=3 biologically independent samples. (c), Relative peptide amounts of CaMKII-δ exon junctions assayed by quantitative mass spectrometry in human heart immunoprecipitated with anti-exon 21 or anti-exon 16. n=3 (left panel) and 8 (right panel) biologically independent samples. (d), Immunofluorescent confocal microscopic images of the cytosolic location of Flag-CaMKII-δ9 (the right upper panel) and HA-CaMKII-δ2 (the left lower panel) in NRVMs infected with Ad-Flag-CaMKII-δ9 and Ad-HA-CaMKII-δ2. Scale bar, 10 μm. n=6 biologically independent samples. (e), (f), CaMKII-δ9 protein levels in NRVMs exposed to 1 μM Doxorubicin (24 h) (e, n=12 (Vehicle) and 10 (Dox) biologically independent samples) or 200 μM H₂O₂ (24 h) (f, n=7 (Vehicle) and 5 (H₂O₂) biologically independent samples). (g), (h), CaMKII-δ9 protein levels in hypertrophic mouse heart (TAC for 2 weeks) (g, n=5 (sham) and 6 (TAC) biologically independent animals) and myocardial tissue from humans with hypertrophic cardiomyopathy (HCM) (h, n=7 (normal humans) and 6 (HCM) biologically independent samples) together with their corresponding controls. Data are mean±s.e.m. One-way ANOVA (c, left panel), two-sided Student's t-test (c, right panel, e-h).

FIG. 2 illustrates that CaMKII-δ9 induces cardiomyocyte death by downregulation of UBE2T. (a), (b), Cellular caspase 3/7 activity in NRVMs treated with scrambled or CaMKII-δ9 siRNAs in the presence or absence of H₂O₂ (200 μM) (a) or Dox (1 μM) (b). n=6 biologically independent samples. (c), Cellular caspase 3/7 activity in NRVMs infected with Ad-β-gal, Ad-CaMKII-δ9, and Ad-CaMKII-δ2 at the indicated MOI for 48 h. n=6 (Ad-β-gal and Ad-CaMKII-δ9), and 4 (Ad-CaMKII-δ2) biologically independent samples. (d), Averaged data of the mRNA levels assayed by real-time PCR of the 3 genes that were upregulated by CaMKII-δ9, but not CaMKII-δ2, in NRVMs infected with Ad-β-gal, Ad-CaMKII-δ9, or Ad-CaMKII-δ2 (MOI 50, 48 h). n=14 biologically independent samples. (e), Representative western blots and statistical data showing that CaMKII-δ9, but not δ2, decreased UBE2T in a dose-dependent manner. n=8 biologically independent samples. (f), Representative western blots and statistical data showing the expression of UBE2T in NRVMs transfected with scrambled or CaMKII-δ9 siRNA. n=5 biologically independent samples. (g), Cellular caspase 3/7 activity in NRVMs infected with Ad-β-gal and Ad-CaMKII-δ9 (MOI 50, 48 h) with or without UBE2T overexpression. n=5 biologically independent samples. (h), Cellular caspase 3/7 activity in NRVMs treated with scrambled or UBE2T siRNAs for 60 h. n 5 biologically independent samples. (i), Representative western blots and statistical data showing the expression of UBE2T in NRVMs with or without H₂O₂ (200 μM). n=5 biologically independent samples. (j), Cellular caspase 3/7 activity in NRVMs with or without UBE2T overexpression and exposed to H₂O₂ (200 μM). n=8 biologically independent samples. Data are mean±s.e.m. Two-way ANOVA (a, b, g, j), one-way ANOVA (c, d, e, h), or two-sided Student's t-test (f, i).

FIG. 3 illustrates that CaMKII-δ9 induces cardiomyocyte DNA damage and genome instability by disrupting UBE2T-mediated DNA repair. (a), Representative immunostaining and statistical data of γH2AX-positive NRVMs infected with Ad-β-gal, Ad-CaMKII-δ2, or Ad-CaMKII-δ9 (MOI 50, 48 h). n=6 biologically independent samples. Arrows indicate γH2AX-positive nuclei. Scale bar, 20 μm. (b), Representative western blots and statistical data showing CaMKII-δ9 increases γH2AX dose-dependently in NRVMs. n=10 biologically independent samples. (c), DNA damage assessed by comet assays in NRVMs infected with Ad-β-gal, Ad-CaMKII-δ2, or Ad-CaMKII-δ9 (MOI 50, 48 h). n=6 biologically independent samples. Arrows indicate nuclei with DNA damage. Scale bar, 20 μm. (d), (e), DNA damage assessed by γH2AX immunostaining (d) and comet assays (e) in NRVMs treated with scrambled or CaMKII-δ9 siRNAs with or without H₂O₂ (100 μM for 10 min). n=6 biologically independent samples. (f), (g), DNA damage assessed by γH2AX immunostaining (f) and comet assays (g) in NRVMs infected with Ad-β-gal or Ad-UBE2T with or without CaMKII-δ9 overexpression (MOI 50, 48 h). n=6 biologically independent samples. (h), (i), DNA damage assessed by γH2AX immunostaining (h) and comet assays (i) in NRVMs infected with Ad-β-gal or Ad-UBE2T with or without H₂O₂ (100 μM for 10 min). n=6 biologically independent samples. (j), (k), DNA damage assessed by γH2AX immunostaining (j) and comet assays (k) in NRVMs treated with scrambled or UBE2T siRNAs for 60 h. n=6 biologically independent samples. (l), (m), Representative western blots and statistical data showing the levels of H2AX in NRVMs infected with scrambled, FANCD2 (1) or FANCI (m) siRNAs. n=4 biologically independent samples. Data are mean±s.e.m. One-way ANOVA (a, b, c, j, k, l, m), or two-way ANOVA (d-i).

FIG. 4 illustrates enhanced CaMKII-δ9-UBE2T-DNA damage signaling in cardiomyopathy and heart failure. (a-d), Myocardial CaMKII-δ9 protein levels (a, n=8 biologically independent animals), Kaplan-Meier survival curves (b, n=20 (wt) and 22 (CaMKII-δ9 tg) biologically independent animals), cardiac gross morphology (c, n=15 (wt) and 9 (CaMKII-δ9 tg) biologically independent animals), and statistical data of cardiac TUNEL staining (d, n=5 biologically independent animals) of wt and CaMKII-δ9 tg mice. Scale bar, 2 mm. (e), (f), Representative echocardiographic images (e) and statistical data (f) of wt and CaMKII-δ9 tg mice at the ages of 6 and 10 weeks. n=13 (wt 6 weeks), 15 (wt 10 weeks), 15 (CaMKII-δ9 tg 6 weeks), and 9 (CaMKII-δ9 tg 10 weeks) biologically independent animals. EF, ejection fraction; FS, fractional shortening; LVIDd and LVIDs, diastolic and systolic left ventricular internal diameter; LVPWd and LVPWs, diastolic and systolic left ventricular posterior wall thickness. (g), Statistical data of cardiac γH2AX staining of wt and CaMKII-δ9 tg mice at the age of 10 weeks. n=6 biologically independent animals. (h), Cardiac UBE2T protein levels of wt and CaMKII-δ9 tg mice at the age of 10 weeks. n=5 (wt), and 6 (CaMKII-δ9 tg) biologically independent animals. (i), CaMKII-δ9 protein levels in the hearts of wt and CaMKII-δ9 shRNA transgenic (shRNA tg) mice at the age of 10 weeks. n=14 (wt) and 11 (shRNA tg) biologically independent animals. (j-m), Statistical data of the EF and FS (j, n=10 (sham), and 16 (wt TAC), and 9 (shRNA tg TAC) biologically independent animals, Kaplan-Meier survival curves (k, n=17 (wt), and 28 (shRNA tg) biologically independent animals, cardiac γH2AX (1, n=5 biologically independent animals) and TUNEL (m, n=5 biologically independent animals) staining of wt and shRNA tg mice 4 weeks after TAC surgery. Data are mean±s.e.m. Two-sided Student's t-test (a, d, g-i, l, m), log-rank (Mantel-Cox) test (b, k), or two-way ANOVA (f, j).

FIG. 5 illustrates that overexpression of UBE2T attenuates CaMKII-δ9-induced DNA damage, cardiomyocyte death and cardiomyopathy. (a), Schematic of the construction of UBE2T tg mice. (b), Kaplan-Meier survival curves of wt and CaMKII-69 tg mice crossed with wt and UBE2T tg mice. n=6 (wt+wt), 5 (wt+UBE2T tg), 22 (CaMKII-39 tg+wt), and 8 (CaMKII-δ9 tg+UBE2T tg) biologically independent animals. c, Left ventricular ejection fraction (EF) and fractional shortening (FS) evaluated by echocardiography of wt and CaMKII-δ9 tg mice crossed with wt and UBE2T tg mice. n=6 (wt+wt), 5 (wt+UBE2T tg), 10 (CaMKII-δ9 tg+wt), and 7 (CaMKII-δ9 tg+UBE2T tg) biologically independent animals. (d), (e), Statistical data of cell death indexed by TUNEL positive cells (d) and DNA damage evidenced by γH2AX positive cells (e) in hearts from wt and CaMKII-δ9 tg mice crossed with wt and UBE2T tg mice. n=5 biologically independent animals. (f), Representative western blots and statistical data showing the levels of UBE2T from the hearts of wt and CaMKII-δ9 tg mice crossed with wt and UBE2T tg mice. n=4 biologically independent animals. Data are mean s.e.m. Log-rank (Mantel-Cox) test (b), or two-way ANOVA (c-f).

FIG. 6 illustrates increased CaMKII-δ9-UBE2T-DNA damage signaling in myocardium from patients with hypertrophic cardiomyopathy and human cardiomyocytes treated with doxorubicin. (a-c), Representative western blots and statistical data of cleaved caspase 3 (a), UBE2T (b), and γH2AX (c) in myocardial tissue from humans with hypertrophic cardiomyopathy (HCM) or normal controls. n=4 (normal human), and 8 (HCM) biologically independent samples. (d-f), Cell viability assayed by caspase 3/7 activity (d), and representative western blots and statistical data showing the levels of γH2AX (e) and UBE2T (f) in human embryonic stem cell-derived cardiomyocytes infected with Ad-β-gal, Ad-CaMKII-δ9, or Ad-CaMKII-δ2 (MOI 100, 48 h). n=4 biologically independent samples. (g-i), Cell viability assayed by caspase 3/7 activity (g, n=6 biologically independent samples), and representative western blots and statistical data showing the levels of γH2AX (h, n=4 biologically independent samples) and UBE2T (i, n=4 biologically independent samples) in human embryonic stem cell-derived cardiomyocytes infected with scrambled or CaMKII-d9 siRNA with or without doxorubicin (Dox) treatment (1 mM, 24 h). Data are mean±s.e.m. Two-sided student's t-test (a-c), one-way ANOVA (d-f), or two-way ANOVA (g-i).

FIG. 7 illustrates that CaMKII-δ9 increases UBE2T phosphorylation at Ser110 and facilitates its degradation. (a), (b), Representative western blots and statistical data showing that the proteasome inhibitors β-lac (a, 5 μM, n=8 biologically independent samples) and MG132 (b, 10 μM, n=6 biologically independent samples) block CaMKII-δ9-mediated UBE2T degradation and γH2AX upregulation in NRVMs. (c), Representative immunostaining images showing the location of myc-tagged UBE2T in the absence and presence of CaMKII-δ9 with or without MG132 (10 M) in NRVMs. n=6 biologically independent samples. Scale bar, 20 μm. (d), Co-immunoprecipitation of CaMKII-δ9 with UBE2T in NRVMs infected with Ad-Flag-CaMKII-δ9 and Ad-UBE2T-myc. The input represents 6% of the whole cell lysate used for each immunoprecipitation. n=4 biologically independent samples. (e), (f), Representative western blots showing that CaMKII-δ9 increases the serine phosphorylation of UBE2T (e) but not the threonine phosphorylation (f) in NRVMs. n=4 biologically independent samples. (g), Representative western blots and average data showing that UBE2T-S110A, but not WT UBE2T or the UBE2T-S193A mutant, resists CaMKII-δ9-mediated degradation (MOI 50, 48 h). n=4 biologically independent samples. (h), Typical western blots and averaged data illustrating the serine phosphorylation and total levels of UBE2T recombinant protein with or without CaMKII-δ9 or CaMKII-δ2 protein co-incubation in a cell-free system. n=6 biologically independent samples. (i), Co-immunoprecipitation of UBE2T and CaMKII-δ2 in lysates of NRVMs infected with Ad-UBE2T-myc and Ad-HA-CaMKII-δ2. n=4 biologically independently samples. Data are mean±s.e m. Two-way ANOVA (a, b), or one-way ANOVA (g, h).

FIG. 8 illustrates specific phosphorylation of UBE2T by CaMKII-δ9. (a), Co-immunoprecipitation of CaMKII-δ1 with UBE2T in NRVMs infected with Ad-Flag-CaMKII-δ1 and Ad-UBE2T-myc. The input represents 6% of the whole cell lysate used for each immunoprecipitation. n=4 biologically independently samples. (b), Representative western blots and statistical data showing that CaMKII-δ9, but not 51, decreased UBE2T in NRVMs. n=4 biologically independently samples. (c), Co-immunoprecipitation of CaMKII-53 with UBE2T in NRVMs infected with Ad-HA-CaMKII-δ3 and Ad-UBE2T-myc. The input represents 6% of the whole cell lysate used for each immunoprecipitation. n=4 biologically independently samples. (d), Representative western blots and statistical data showing that CaMKII-δ9, but not 53, decreases UBE2T in NRVMs. n=6 biologically independently samples. (e), Co-immunoprecipitation of the peptides encoded by CaMKII-5 exon junctions of exons 13-16-17 with UBE2T in HEK293 cells transfected with plasmids of the corresponding exon junctions (tagged with Flag-GFP), or UBE2T-myc. n=4 biologically independently samples. (f), Schematic presentation showing CaMKII-δ9-mediated cardiac DNA damage and cardiomyocyte death signaling. Under normal conditions, UBE2T guard the genome against various types of DNA damage to maintain the survival of cardiomyocytes. When the cardiomyocytes are subjected to insults, CaMKII-δ9 is upregulated and hyper-activated, and this enhances the Ser110 phosphorylation and subsequent degradation of UBE2T. The decreased UBE2T level impairs the DNA repair machinery, leading to the accumulation of DNA damage, genome instability and cell death. Data are mean±s.e.m. One-way ANOVA.

FIG. 9 illustrates that CaMKII-δ9 is present in the heart. (a), Schematic of CaMKII-5 splice variants. CaMKII-5 mainly undergoes alternative splicing events at two variable domains, one between exons 13 and 17, and the other after exon 20. Exons are numbered, and full-size boxes represent coding exons, while smaller, green boxes stand for untranslated regions (UTRs), with special exons colored. (b), Strategy for SMRT sequencing of full-length CaMKII-δ transcripts. CaMKII-δ transcripts were reverse-transcribed from total RNA isolated from the hearts of different species. Each cDNA was amplified with paired primers, whose locations are marked with arrows of different colors. The forward primer (left) is located on exon 1, and the reverse primer (right) is located on exon 22. The PCR products were concentrated and purified for SMRT sequencing. (c-e), Percentages of exon junctions of CaMKII-δ assayed by RNA-seq in variable domain 1 (between exons 13 and 17 (c) and 14-17 (d)), and variable domain 2 (between exons 20 and 22) (e) in the hearts of human, rhesus monkey, dog, rat, and mouse. In panel e, in the hearts of monkey and human, exon 20b is another form of exon 20, which is 147 bases longer than the classic exon 20, and has not been described before. Data are mean±s.e.m. (n=8 (human and rat), 7 (rhesus monkey), and 6 (dog and mouse) biologically independent samples). Data are mean±s.e.m. One-way ANOVA.

FIG. 10 illustrates the identification of CaMKII-δ9 protein in the heart. (a), Peptides sequences of exons 16 and 21 used as antigens for the production of antibodies. (b), Immunoblots of NRVMs transfected with Ad-β-gal, Ad-HA-CaMKII-δ2, Ad-HA-CaMKII-δ3, or Ad-Flag-CaMKII-δ9, with serum containing anti-exon 16 or anti-exon 21. n=3 biologically independent samples. (c), (d), Western blots of Flag-tagged CaMKII-δ9 recombinant protein with anti-exon 16 (c) and anti-exon 21 (d) with increasing ratios of the corresponding antigen peptide to CaMKII-δ9 protein. n=4 biologically independent samples. Mean±s.e.m. One-way ANOVA. (e), (f), Heart lysates of 10-week old mice immunoprecipitated with anti-exon 21 (e) or anti-exon 16 (f), followed by SDS-PAGE and Coomassie blue staining. The bands at ˜50 kD (boxes) were cut for MS analysis. (g), (h), LC-MS/MS spectra of the peptides matching the junction of CaMKII-δ exons 13-16-17 (g) and exons 20-21 (h) from mouse hearts immunoprecipitated with anti-exon 21 (g) or anti-exon 16 (h). The peptide sequence above is the corresponding exon junction, with the specific exons 16 and 21 in bold. bₙ ions are fragments at the n^{th} peptide bonds that contain the amino-terminal part of the peptide, whereas yₙ ions contain the carboxy-terminal part. NL, normalized intensity level (counts per second). (i), (j), CaMKII-δ9 tissue distribution in rhesus monkeys (i) and wt mice (j). n=4 biologically independent samples. CaMKII-δ9 recombinant protein served as a positive control (PC). In both species, a band with a higher molecular weight was detected in the brain, which was the brain-enriched splice variant CaMKII-δ1 (exons 13-15-16-17). (k), (l), Immunofluorescent confocal microscopic images of the cytosolic location of endogenous CaMKII-δ9 (gray) in adult (left) and neonatal (right) rat ventricular cardiomyocytes (k, n=4 biologically independent samples), and the nuclear location of HA-CaMKII-δ3 (gray) in NRVMs infected with Ad-HA-CaMKII-δ3 (1, n=6 biologically independent samples). Scale bars, 10 μm. (m), CaMKII-δ9 protein levels in nuclear and cytosolic fractions of NRVMs. n=6 biologically independent samples.

FIG. 11 illustrates the pathological relevance of CaMKII-δ9 in the heart. (a), (b), Western blots showing the phosphorylation (a, n=8 biologically independent samples) and oxidation (b, n=7 biologically independent samples) levels of CaMKII-δ9 in NRVMs with or without Dox treatment (1 μM, 30 and 60 min). The NRVMs were infected with Ad-Flag-CaMKII-δ9, and the lysates were immunoprecipitated with Flag antibody and subjected to western blot analysis. (c), (d), Representative western blots and statistical data showing the phosphorylation (c) and oxidation (d) levels of CaMKII-δ9 in perfused mouse hearts with or without I/R injury (30 min ischemia followed by 60 min reperfusion). n=6 biologically independent samples. The heart lysates were immunoprecipitated with exon 16 antibody and subjected to western blot analysis. (e), Sequence of CaMKII-δ9 siRNA. The black sequence is the siRNA target in exon 16 of CaMKII-δ9. The siRNA sequence is below in gray. (f), (g), Knockdown efficiency of CaMKII-δ9 siRNA confirmed by mRNA (f, n=5 (scrambled), and 8 (CaMKII-δ9 siRNA) biologically independent samples) and protein (g, n=3 biologically independent samples) levels. (h), Averaged data of mRNA levels of CaMKII-δ2 and CaMKII-δ3 assayed by real-time PCR in NRVMs infected with scrambled or CaMKII-δ9 siRNA. n=18 (CaMKII-δ2), and 15 (CaMKII-δ3) biologically independent samples. (i), (j), Cell viability assessed by LDH concentration in the culture medium of NRVMs treated with scrambled or CaMKII-δ9 siRNAs with or without H₂O₂ (200 μM) (i) or Dox (1 μM) (j). n=6 biologically independent samples. (k), Viability assessed by LDH in the medium of NRVMs infected with Ad-β-gal, Ad-CaMKII-δ9, and Ad-CaMKII-δ2 at the indicated MOI for 48 h. n=10 biologically independent samples. (l), Representative western blots and statistical data of CaMKII-δ expression in NRVMs infected with Ad-β-gal, Ad-CaMKII-δ9, or Ad-CaMKII-δ2 (MOI 50, 48 h). n=3 biologically independent samples. Data are mean±s.e.m. One-way ANOVA (a, b, g, k), two-sided Student's t-test (c, d, f, h, l), or two-way ANOVA (i, j).

FIG. 12 illustrates the RNA-seq analysis of the gene expression profiles of CaMKII-δ9 and CaMKII-δ2. (a), Heatmap representing the gene expression signature among NRVMs infected with Ad-β-gal, Ad-CaMKII-δ9, or Ad-CaMKII-δ2 (MOI 50, 48 h) based on genes differentially-expressed between Ad-β-gal and Ad-CaMKII-δ9. The expression value of each gene was calculated as fragments per kilobase of transcript, per million mapped fragments (FPKM). Seventy-seven genes are listed; they were differentially and significantly changed (>1.5-fold or <0.67-fold, n=3 biologically independent samples) in cells infected with Ad-CaMKII-δ9 relative to those in cells infected with Ad-β-gal. The 15 genes that were different regulated in Ad-CaMKII-δ9 and Ad-CaMKII-δ2 are in gray. The heatmap was generated by the R package “pheatmap” with the option “scale=row”, which means the expression value of each gene is the z-score normalized by the FPKM value. (b), Data of mRNA levels assayed by real-time PCR of 12 of the 15 genes identified by RNA-seq to be regulated by CaMKII-δ9, but not CaMKII-δ2, in NRVMs infected with Ad-β-gal, Ad-CaMKII-δ9, or Ad-CaMKII-δ2 (MOI 50, 48 h). n=14 biologically independent samples. (c), (d), Protein levels of COX-2 (c) and PAI-2 (d) in cultured NRVMs infected with Ad-β-gal, Ad-CaMKII-δ9, or Ad-CaMKII-δ2 (dose as indicated, 48 h). n=4 biologically independent samples. (e), Typical western blots and averaged data showing the protein level of COX-2 in NRVMs transfected with scrambled or COX-2 siRNAs. n=4 biologically independent samples. (f), (g), Cell viability indexed by caspase 3/7 activity (f) and LDH concentration in the culture medium (g) in NRVMs infected with Ad-β-gal or Ad-CaMKII-δ9 in the presence or absence of COX-2 siRNAs. n=3 (f), and 6(g) biologically independent samples. (h), Representative western blots and averaged data showing the protein levels of UBE2T in NRVMs transfected with scrambled or UBE2T siRNAs. n=4 biologically independent samples. Data are mean±s.e.m. One-way ANOVA (b, c, d, e, h), or two-way ANOVA (f, g).

FIG. 13 illustrates that CaMKII-δ9 induced cardiomyocyte DNA damage. (a), (b), Complete representative immunostaining of γ-H2AX (a) and comet assays (b) in NRVMs infected with Ad-β-gal, Ad-CaMKII-δ2, or Ad-CaMKII-δ9 (MOI 50, 48 h). Scale bars, 20 μm. Part of the representative images and averaged data are shown in FIG. 3a, c. (c-f), Representative western blots and statistical data showing the levels of FANCD2 (c, n=4 biologically independent samples) and FANCI (d, n=4 biologically independent samples), and caspase 3/7 activity (e, f, n=5 biologically independent samples) in NRVMs infected with scrambled, FANCD2 or FANCI siRNAs. Data are mean±s.e.m. One-way ANOVA.

FIG. 14 illustrates the role of CaMKII-δ9-UBE2T signaling in cardiac pathophysiology. (a), Schematic of the construction of CaMKII-δ9 tg mice. (b), PCR genotyping of wt and CaMKII-δ9 tg mice. (c), (d), Ratio of heart weight to body weight (c, n=15 (wt), and 9 (CaMKII-δ9 tg) biologically independent samples) and ventricular gene expression (d, n=7 (wt), and 6 (CaMKII-δ9 tg) biologically independent samples) of wt and CaMKII-δ9 tg mice at the age of 10 weeks. (e), Immunostaining of cardiac γ-H2AX of wt and CaMKII-δ9 tg mice at the age of 10 weeks. Arrows, γ-H2AX-positive cells. Right window, enlarged image of the view as indicated. Averaged data are in FIG. 4g. Scale bar, 20 M. (f), Schematic of the construction of CaMKII-δ9 shRNA transgenic (shRNA tg) mice. (g), PCR genotyping of wt and shRNA tg mice. (h), Cardiac exon 21 protein levels of wt and shRNA tg mice at the age of 10 weeks (n=4 biologically independent samples). (i), (j), Ratio of heart weight to body weight (i, n=16 (wt), and 8 (shRNA tg) biologically independent samples), and cardiac UBE2T protein levels (j, n=4 biologically independent samples) of wt and shRNA tg mice 4 weeks after TAC surgery. (k), Cardiac CaMKII-δ protein levels of CaMKII-δ9 tg and CaMKII-δ2 tg mice at the age of 10 weeks (n=4 biologically independent samples). (l-q), Cardiac TUNEL staining (1, n=8 biologically independent samples), echocardiography (m, n=8 biologically independent samples), ratio of heart weight to body weight (n, n=8 biologically independent samples), Kaplan-Meier survival curves (o, n=10 (wt), 19 (CaMKII-δ9 tg), and 12 (CaMKII-δ2 tg) biologically independent samples), cardiac γ-H2AX staining (p, n=8 biologically independent samples), and cardiac UBE2T protein levels (q, n=4 biologically independent samples) from wt, CaMKII-δ9 tg and CaMKII-δ2 tg mice. Data are mean±s.e.m. Two-sided Student's t-test (c, d, i), one-way ANOVA (k-n, p, q), two-way ANOVA (j), or log-rank (Mantel-Cox) test (o).

FIG. 15 illustrates that CaMKII-δ9 phosphorylates UBE2T at Ser110 site. (a), Mass spectrometric data showing two potential phosphorylation sites (Ser110 and Ser193, circles) of UBE2T mediated by CaMKII-δ9 (n=3 biologically independent samples). HEK293 cells were transfected with myc-tagged UBE2T in the presence of control vector or Flag-tagged CaMKII-δ9, whole-cell lysates were immunoprecipitated with myc antibody, and then the immuno-complex was subjected to post-translational modification mass spectrometric analysis. (b), Sequence alignment of UBE2T protein showing conservation of the Ser110 site, but not the Ser193 site of UBE2T (arrows) in 12 species. (c), Typical western blots and averaged data showing the serine phosphorylation and total levels of UBE2T and UBE2T-S110A recombinant protein with or without CaMKII-δ9 protein co-incubation in a cell-free system (n=4 biologically independent samples). Two-way ANOVA. (d), Representative immunofluorescence images of wt UBE2T, UBE2T-S110A, and UBE2T-S193A (all with myc tag) in NRVMs infected with Ad-UBE2T, Ad-UBE2T-S110A, or Ad-UBE2T-S193A (n=6 biologically independent samples). Note that UBE2T-S110A was resistant to degradation, and distributed in both cytoplasm and nucleus, indicating that the Ser110 site of UBE2T is responsible for its degradation, but not its subcellular distribution, Scale bar, 10 μm. Data are mean±s.e.m.

FIG. 16 illustrates the interaction between the peptides encoded by CaMKII-δ exon junctions and UBE2T. (a-c), Co-immunoprecipitation of the peptides encoded by CaMKII-5 exon junctions of exons 13-17 (a), 13-14-17 (b), and 13-15-16-17 (c), with UBE2T in HEK293 cells transfected with plasmids of the corresponding exon junctions (tagged with Flag-GFP), or UBE2T-myc. (n=4 biologically independently samples).

FIG. 17 illustrates the amino acid sequence (SEQ ID NO: 1) of exon 16 of CaMKII-δ gene, the amino acid sequence (SEQ ID NO: 2) of exons 13-16 of CaMKII-δ gene, the amino acid sequence (SEQ ID NO: 3) of exons 16-17 of CaMKII-δ gene, the amino acid sequence (SEQ ID NO: 4) of exons 13-16-17 of CaMKII-δ gene, and the full-length amino acid sequence (SEQ ID NO: 5) of CaMKII-δ9.

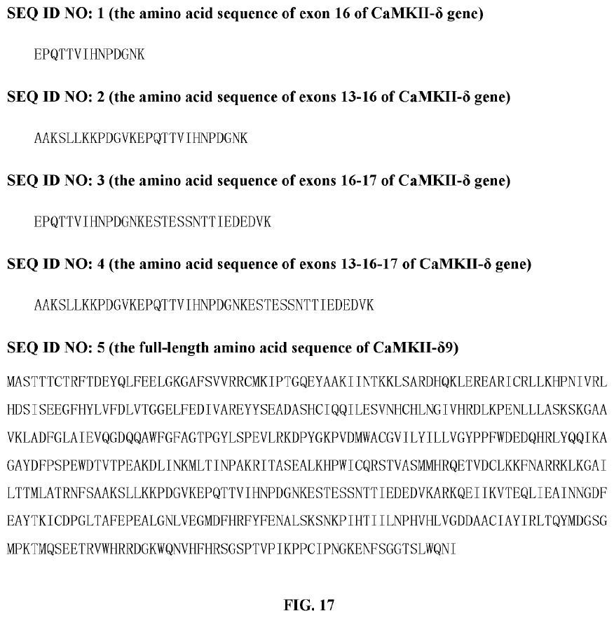

FIG. 18 illustrates the nucleic acid sequence (SEQ ID NO: 6) of human and rat exon 16 of CaMKII-δ gene, the nucleic acid sequence (SEQ ID NO: 7) of mouse exon 16 of CaMKII-δ gene, the nucleic acid sequence (SEQ ID NO: 8) of human exons 13-16 of CaMKII-δ gene, the nucleic acid sequence (SEQ ID NO: 9) of rat exons 13-16 of CaMKII-δ gene, the nucleic acid sequence (SEQ ID NO: 10) of mouse exons 13-16 of CaMKII-5 gene, the nucleic acid sequence (SEQ ID NO: 11) of human exons 16-17 of CaMKII-δ gene, the nucleic acid sequence (SEQ ID NO: 12) of rat exons 16-17 of CaMKII-δ gene, the nucleic acid sequence (SEQ ID NO: 13) of mouse exons 16-17 of CaMKII-5 gene, the nucleic acid sequence (SEQ ID NO: 14) of human exons 13-16-17 of CaMKII-δ gene, the nucleic acid sequence (SEQ ID NO: 15) of rat exons 13-16-17 of CaMKII-δ gene, the nucleic acid sequence (SEQ ID NO: 16) of mouse exons 13-16-17 of CaMKII-δ gene.

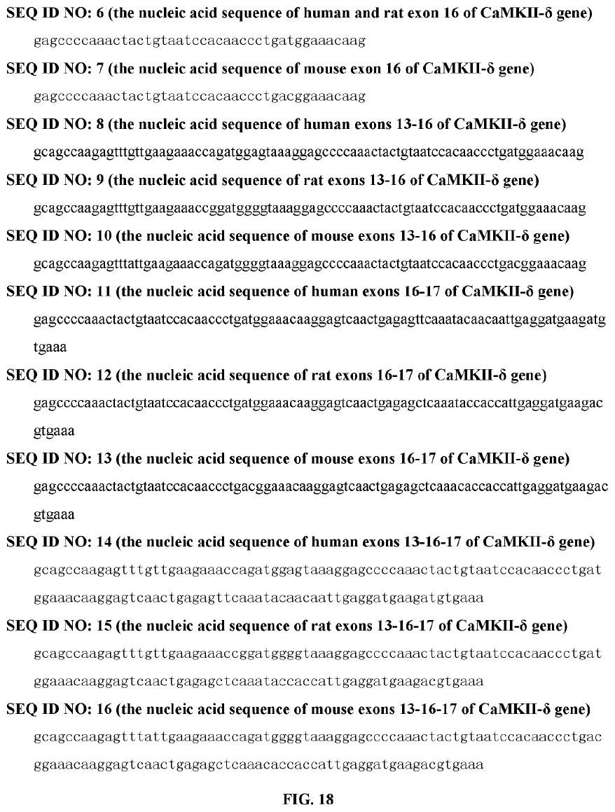

FIG. 19 illustrates the nucleic acid sequence (SEQ ID NO: 17) of the full-length human CaMKII-δ9).

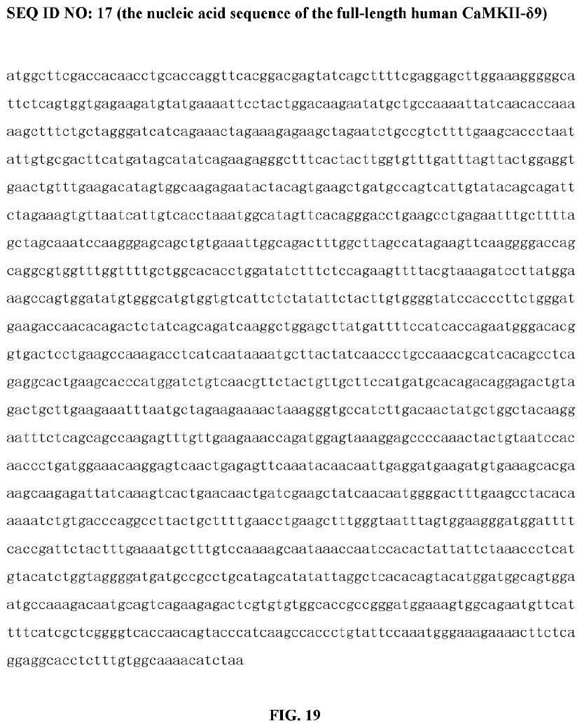

FIG. 20 illustrates the nucleic acid sequence (SEQ ID NO: 18) of the full-length rat CaMKII-δ9).

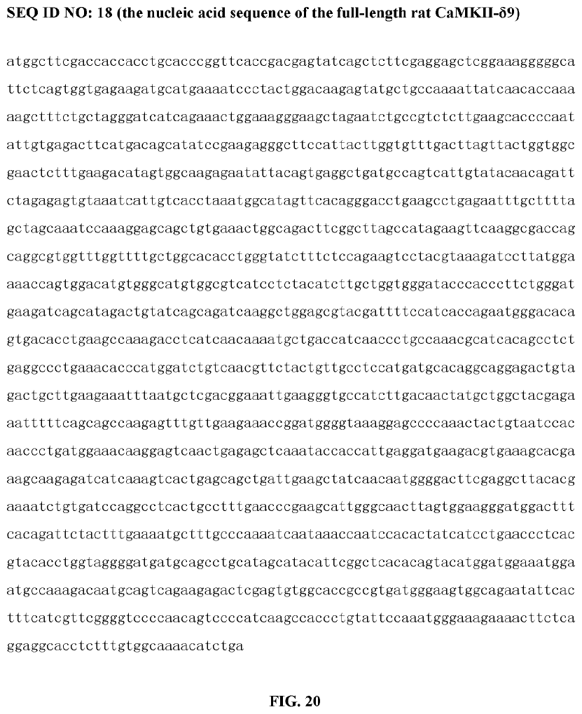

FIG. 21 illustrates the nucleic acid sequence (SEQ ID NO: 19) of the full-length mouse CaMKII-δ9).

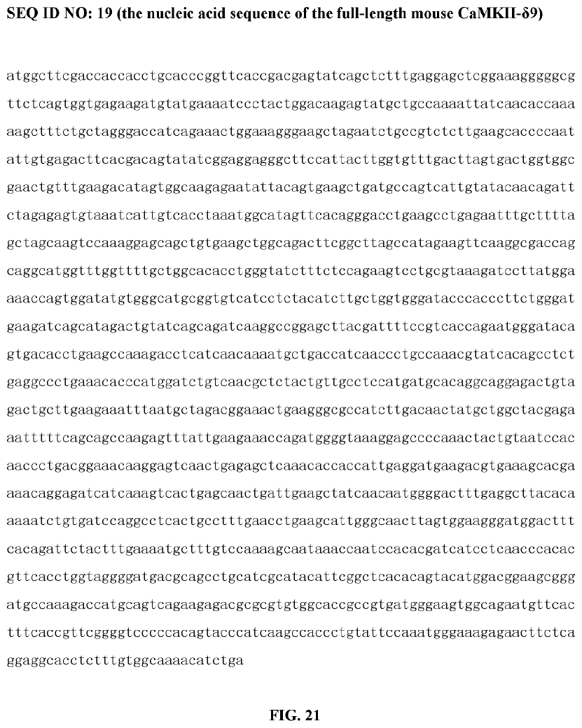

### DETAILED DESCRIPTION

Before the present invention is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the present invention, subject to any specifically excluded limit in the stated range. Where the stated range includes one or both of the limits, ranges excluding either or both of those included limits are also included in the present invention.

Unless defined otherwise, all technical and scientific terms used herein have the same meaning as commonly understood by those of skill in the art to which the present invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide those of skill in the art with a general guide to many of the terms used in the present invention. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

Embodiments of the present invention will employ, unless otherwise indicated, techniques of chemistry, solid state chemistry, inorganic chemistry, organic chemistry, physical chemistry, analytical chemistry, materials chemistry, biochemistry, biology, molecular biology, recombinant DNA techniques, pharmacology, imaging, and the like, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” series (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Primers, polynucleotides and polypeptides employed in the present invention can be generated using standard techniques known in the art.

The following embodiments are put forth so as to provide those of skill in the art with a complete disclosure and description of how to perform the methods and use the biomarkers and kits disclosed and claimed herein. Efforts have been made to ensure accuracy with respect to numbers (e.g., amounts, temperature, etc.), but some errors and deviations should be accounted for.

It must be noted that, as used in the specification and the appended claims, the singular forms “a,” “an,” and “the” include plural forms of the same unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

In one aspect, the present invention discloses methods of treating or preventing a CaMKII-mediated disease in a subject, comprising administering to the subject an effective amount of an antagonist of CaMKII-δ9. In another aspect, the present invention discloses use of an antagonist of CaMKII-δ9 in the manufacture of a medicament for treating or preventing a CaMKII-mediated disease in a subject. In yet another aspect, the present invention discloses an antagonist of CaMKII-δ9 for use in treating or preventing a CaMKII-mediated disease in a subject.

As used herein, the terms “treat”, “treating” or “treatment” refer to clinical intervention in an attempt to alter the natural course of a disease, condition or disorder in a subject being treated, and can be performed either for prophylaxis or during the course of clinical pathology. Desirable effects of treatment include prevention of occurrence or recurrence of the disease, condition or disorder, alleviation of symptoms, diminishment of any direct or indirect pathological consequences of the disease, condition or disorder, decrease of the rate of progression of the disease, condition or disorder, amelioration or palliation of the disease state, and remission or improved prognosis.

As used herein, the terms “prevent”, “preventing” or “prevention” refer to reducing the probability of developing a disease, condition or disorder in a subject, who does not have, but is at risk of or susceptible to developing a disease, condition or disorder.

As used herein, the term “CaMKII-mediated disease” refers to a disease, condition or disorder that is associated with the abnormal level and/or activity of CaMKII, either caused or facilitated by abnormal high or low level and/or activity of CaMKII due to abnormal activation or destroy of CaMKII in a subject. In some embodiments, the CaMKII-mediated disease is associated with an increased level and/or activity of CaMKII-δ9. In some embodiments, the CaMKII-mediated disease is a heart disease or a metabolic disease. In some embodiments, the heart disease is selected from the group consisting of cardiomyopathy, myocarditis, diabetic heart disease, myocardial ischemia, cardiac ischemia/reperfusion injury, myocardial infarction, heart failure, arrhythmia, heart rupture, angina, cardiac hypertrophy, cardiac injury, hypertensive heart disease, rheumatic heart disease, angina, myocarditis, coronary heart disease and pericarditis. In some embodiments, the heart disease is hypertrophic cardiomyopathy. In some embodiments, the metabolic disease is selected from the group consisting of insulin resistance, obesity, diabetes, hypertension, dyslipidemia, diabetic cerebrovascular diseases, diabetic ocular complications, diabetic neuropathy, diabetic foot, hyperinsulinemia, hypercholesterolemia, hyperglycaemia, hyperlipemia, gout and hyperuricemia.

As used herein, the terms “administer”, “administering”, “administered” and “administration” refer that the substance is delivered to a subject in need thereof. The route of administration may be topical, oral, intranasal, parenteral, enteric, rectal, intravenous, intraperitoneal, subcutaneous, pulmonary, transdermal, intramuscular, buccal, sublingual, or ocular. In some embodiments, the substance can be administered to a subject using peripheral systemic delivery by intravenous, intraperitoneal, or subcutaneous injection.

As used herein, the term “subject” includes both human and non-human animals. Non-human animals include all vertebrates, such as mammals and non-mammals. The “subject” may also be a domestic animal such as cow, swine, sheep, poultry and horse; or rodent such as rat, mouse; or a non-human primate such as ape, monkey, rhesus monkey; or domesticated animal such as dog or cat. The term “subject” does not aim to be limiting in any aspect, and can be of any age, sex and physical condition, for example, may be male or female, and may be elderly, adult, adolescent, child or infant. A human “subject” may be Caucasian, African, Asian, Semitic, or other races, or a mixture of the racial backgrounds above. In some embodiments, the subject is a human or non-human primate. In some embodiments, the non-human primate is a rhesus monkey. In some embodiments, the subject is a human.

As used herein, the term “effective amount” refers to the amount of a medicament which achieves a therapeutic effect or prophylactic effect by inhibiting or alleviating a disease, condition or disorder of a subject, or by prophylactically inhibiting or preventing the onset of a disease, disorder or symptoms. An effective amount may be the amount of the medicament which relieves to some extent one or more symptoms of a disease or disorder in a subject; returns to normal either partially or completely one or more physiological or biochemical parameters associated with or causative of the disease or disorder; and/or reduces the likelihood of the onset of the disease or disorder. A clinician skilled in the art can determine the effective amount of a medicament in order to treat or prevent a particular disease or disorder when it is administered. The precise amount of the medicament required to be effective will depend upon numerous factors, e.g., such as the specific activity of the active substance, the delivery device employed, physical characteristics of the substance, purpose for the administration, in addition to many patient specific considerations. The determination of amount of a medicament that must be administered to be an effective amount is routine in the art and within the skill of an ordinarily skilled clinician.

As used herein, the term “antagonist” refers to a molecule that inhibits the expression level or activity of a protein, polypeptide or peptide, thereby reducing the amount, formation, function, and/or downstream signaling of the protein, polypeptide or peptide. For example, “antagonist of CaMKII-δ9” of the present invention refers to a molecule that inhibits the expression level or activity of CaMKII-δ9, thereby reducing the amount, formation, function, and/or downstream signaling of CaMKII-δ9.

A molecule is considered to inhibit the expression level or activity of CaMKII-δ9 if the molecule causes a significant reduction in the expression (either at the level of transcription or translation) level or activity of CaMKII-δ9. Similarly, a molecule is considered to inhibit the binding between CaMKII-δ9 and its substrate if the molecule causes a significant reduction in the binding between CaMKII-δ9 and its substrate, which causes a significant reduction in downstream signaling and functions mediated by CaMKII-δ9 (e.g., the reduction of phosphorylation of ubiquitin-conjugating enzyme). A reduction is considered significant, for example, if the reduction is at least about 10%, 20%, 30%, 40%, 50%, 60%, 70%, 80%, 90%, 91%, 92%, 93%, 94%, 95%, 96%, 97%, 98%, or 99%.

A binding antagonist can act in two ways. In some embodiments, a binding antagonist of the present invention can compete with CaMKII-δ9 to bind to its substrate and thereby interfering with, blocking or otherwise preventing the binding of CaMKII-δ9 to its substrate. This type of antagonist, which binds the substrate but does not trigger the expected signal transduction, is also known as a “competitive antagonist” and can include, for example, a vector that expresses CaMKII-δ9 which is without phosphorylation or oxidation function. In other embodiments, a binding antagonist of the present invention can bind to and sequester CaMKII-δ9 with sufficient affinity and specificity to substantially interfere with, block or otherwise prevent binding of CaMKII-δ9 to its substrate. This type of antagonist is also known as a “neutralizing antagonist”, and can include, for example, an antibody or aptamer directed to CaMKII-δ9 which specifically binds to CaMKII-δ9.

In some embodiments, the antagonist is an antagonist for inhibiting the phosphorylation of ubiquitin-conjugating enzyme. In some embodiments, the ubiquitin-conjugating enzyme is UBE2T. In some embodiments, the antagonist is an antagonist for inhibiting the phosphorylation of UBE2T at Ser110.

Ubiquitination regulates degradation of cellular proteins by the ubiquitin proteasome system, controlling a protein's half-life and expression levels. This process involves the sequential action of ubiquitin-activating enzymes (E1), ubiquitin-conjugating enzymes (E2), and ubiquitin ligases (E3). Ubiquitin-conjugating enzymes perform the second step in the ubiquitination reaction that targets a protein for degradation via the proteasome. Phosphorylation is a biochemical reaction in which a phosphate group is added to Serine (Ser), Threonine (Thr) or Tyrosine (Tyr) residues of a protein and is catalyzed by protein kinase enzymes. For example, CaMKII-δ9 phosphorylates UBE2T at Ser110. Phosphorylation normally modifies the functions of target proteins, often causing activation. As part of the cell's homeostatic mechanisms, phosphorylation is only a transient process that is reversed by other enzyme called phosphatases. Therefore, protein phosphorylation levels change over time and can be evaluated in a number of well-known manners, including, for example, by immunological approaches. For example, the amount of phosphorylated UBE2T is determined by an immunoassay using a reagent which specifically binds with phosphorylated UBE2T. Such an immunoassay can have a number of well-known forms, including, without limitation, a radioimmunoassay, a Western blot assay, an immunofluorescence assay, an enzyme immunoassay, an immunoprecipitation assay, a chemiluminescence assay, an immunohistochemical assay, a dot blot assay, or a slot blot assay.

In some embodiments, the enzyme immunoassay is a sandwich enzyme immunoassay using a capture antibody or fragment thereof which specifically binds with UBE2T and a detection antibody or fragment thereof which specifically binds with phosphorylated UBE2T. Such an enzyme immunoassay is particularly advantageous because identifying differences in protein levels between related kinase family members or isoforms gives the relatively high homology between kinases among themselves and also among their phosphorylated forms.

Immunological reagents for identifying UBE2T in both phosphorylated and non-phosphorylated forms, as well as for detecting CaMKII-δ9, are well known in the art and can be generated using standard techniques, such as by inoculating host animals with appropriate fragments of UBE2T to generate antibodies (e.g. monoclonal antibody) against CaMKII-δ9, UBE2T, and/or phospho-UBE2T. Such anti-CaMKII-δ9, anti-UBE2T, and/or anti-phospho-UBE2T antibody reagents can be used to isolate and/or determine the amount of the respective proteins such as in a cellular lysate. Such reagents can also be used to monitor protein levels in a cell or tissue, e.g., white blood cells or lymphocytes, as part of a clinical testing procedure, e.g., in order to monitor an optimal dosage of an inhibitory agent. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵, ¹³¹I, ³⁵S or ³H.

In some embodiments, the antagonist is a specific antagonist of CaMKII-δ9.

As used herein, the term “specific antagonist” means that the antagonist should not significantly inhibit any peptide, polypeptide or substance, other than CaMKII-δ9. In some embodiments, the specific antagonist should have at least 3 times, 10 times, 20 times, 30 times, 40 times, or 50 times higher inhibition effect against CaMKII-δ9 than against any other relevant peptide or polypeptide. For example, the antagonist has at least 3 times, 5 times, 10 times, 15 times, 20 times, 25 times, 30 times, 35 times, 40 times, 45 times, 50 times higher inhibition effect against CaMKII-δ9 than against CaMKII-δ2 or CaMKII-δ3. In some embodiments, the antagonist inhibits the level or activity of CaMKII-δ9 but does not significantly inhibit the level or activity of CaMKII-δ2 or CaMKII-δ3. As used herein, the term “significantly” refers to statistically significant differences, or significant differences that can be recognized by those of skill in the art.

In some embodiments, the antagonist is an antibody that specifically recognizes CaMKII-δ9.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof), It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL nucleic acid sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, biomarker peptide and a C-terminal polyhistidine tag to make bivalent and biotinylated scFv polypeptides (Kipriyanov, S. M., et a. (1994)Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. In some embodiments, antibodies of the present invention bind specifically or substantially specifically to CaMKII-δ9 or fragment thereof.

The terms “monoclonal antibodies” and “monoclonal antibody”, as used herein, refer to a population of antibody polypeptides that contain only one species of an antigen binding site capable of immunoreacting with a particular epitope of an antigen, whereas the term “polyclonal antibodies” and “polyclonal antibody” refer to a population of antibody polypeptides that contain multiple species of antigen binding sites capable of interacting with a particular antigen. A monoclonal antibody typically displays a single binding affinity for a particular antigen with which it immunoreacts. In some embodiments, a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, the term “specifically recognize(s)” means that the antibody should not bind substantially to (“cross-react” with) another peptide, polypeptide or substance. In some embodiments, the specifically recognized peptide or polypeptide should be bound with at least 3 times, 10 times, 20 times, 30 times, 40 times, or 50 times higher affinity than any other relevant peptide or polypeptide. For example, the antagonist specifically binds to CaMKII-δ9 with at least 3 times, 5 times, 10 times, 15 times, 20 times, 25 times, 30 times, 35 times, 40 times, 45 times, 50 times higher affinity than CaMKII-δ2 or CaMKII-δ3. In some embodiments, the antagonist inhibits the activity of CaMKII-δ9 but does not significantly inhibit the activity of CaMKII-δ2 or CaMKII-δ3.

As used herein, the terms “inhibit”, “inhibiting” and “inhibition” refer to a decrease in the baseline activity of a biological activity or process. “Inhibition of activity of CaMKII-δ9” refers to a decrease in level or activity of CaMKII-δ9 as a direct or indirect response to the presence of the antagonist of the present invention relative to the level or activity of CaMKII-δ9 in the absence of the antagonist of the present invention. In some embodiments, the activity of CaMKII-δ9 comprises the phosphorylation and/or oxidation activity of CaMKII-δ9, which can be measured by those of skill in the art.

In some embodiments, the antibody binds to the amino acid sequence encoded by exon 16 of CaMKII-δ gene. In some embodiments, the antibody binds to the amino acid sequence encoded by exons 13-16 of CaMKII-5 gene. In some embodiments, the antibody binds to the amino acid sequence encoded by exons 16-17 of CaMKII-δ gene. In some embodiments, the antibody binds to the amino acid sequence encoded by exons 13-16-17 of CaMKII-δ gene. In some embodiments, the antibody binds to the amino acid sequence of the full-length CaMKII-δ9.

As used herein, the term “amino acid” in its broadest sense, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid; in some embodiments, an amino acid is a standard amino acid; in some embodiments, an amino acid is a nonstandard amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “encoded” or “encoding” means capable of transcription into mRNA and/or translation into a peptide or protein. The term “encoding sequence” or “gene” refers to a polynucleotide sequence encoding a peptide or protein. These two terms can be used interchangeably in the present invention. In some embodiments, the encoding sequence is a complementary DNA (cDNA) sequence that is reversely transcribed from a messenger RNA (mRNA). In some embodiments, the encoding sequence is mRNA.

In some embodiments, the exon 16, exons 13-16, exons 16-17 and exons 13-16-17 of CaMKII-δ gene, and the nucleic acid sequence of the full-length CaMKII-δ9 comprise the nucleic acid sequence that has at least 70% homology, e.g., at least 75%, at least 80%, at least 85%, at least 90%, at least 95%, or at least 99% homology to any one of the nucleic acid sequences set forth in SEQ ID NOs: 6-19, and can still encode one of the amino acid sequences set forth in SEQ ID NOs: 1-5.

In some embodiments, CaMKII-δ9 has the amino acid sequences set forth in SEQ ID NOs: 1-5. In some embodiments, the encoding sequences of CaMKII-δ9 has the nucleic acid sequences set forth in SEQ ID Nos: 6-19. In some embodiments, the present invention provides nucleic acid sequences encoding SEQ ID NOs: 1-5, but they are different from any one of the nucleic acid sequences set forth in SEQ ID NOs: 6-19 due to the degeneracy of the genetic code.

As used herein, the term “degeneracy of the genetic code” refers to a phenomenon that one amino acid has two or more corresponding genetic codons. For example, proline has 4 synonymous codons CCU, CCC, CCA, and CCG. It is well-known in the art that due to the degeneracy of genetic codes, it is possible to replace nucleic acids in certain positions in a given nucleic acid sequence without changing the encoded amino acid sequence. It is trivial for those of skill in the art to conduct the replacement of degeneracy of the genetic code by, for example, the site-directed mutagenesis of bases. Different organisms have developed different preferences for different codons. In order to express the polypeptide of the present invention in a selected biological cell, the preferred codon of the biological cell can be selected to obtain the corresponding coding sequence, and the amino acid sequences (e.g., SEQ ID NOs: 1-5) of the present invention can be obtained by recombinant expression.

In some embodiments, the antagonist is a small molecule compound that binds to CaMKII-δ9.

As used herein, the term “small molecule compound” means a low molecular weight compound that may serve as an enzyme substrate or regulator of biological processes. In general, a “small molecule compound” is a molecule that is less than about 5 kilodaltons (kD) in size. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

In some embodiments, the antagonist is an RNAi (RNA interference) molecule that targets an encoding sequence of CaMKII-δ9 or an antisense nucleotide that targets an encoding sequence of CaMKII-δ9. In some embodiments, the RNAi molecule is a small interfering RNA (siRNA), a small hairpin RNA (shRNA) or a microRNA (miRNA).

RNAi of the present invention may include one or more than one type of nucleic acid molecules having specificity towards CaMKII-δ9. For example, only one type of siRNA may be used to down-regulate CaMKII-δ9; two types of siRNA (e.g., with different sequences) may be used in combination to modulate expression level of CaMKII-δ9; an antisense oligonucleotide may be combined with an siRNA to reduce the level of CaMKII-δ9.

RNAi of the present invention down-regulates CaMKII-δ9 at various levels, such as post-transcriptional level, pre-transcriptional level, or epigenetic level. In a non-limiting example, epigenetic regulation of gene expression by RNAi molecules of the invention can result from RNAi mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

The RNAi molecule of the present invention can be double-stranded or single-stranded. When the RNAi is double-stranded, one strand is the sense strand and the other is the antisense strand; the antisense strand comprises nucleotide sequence that is complementary to the encoding sequence of CaMKII-δ9 or a portion thereof, and the sense strand comprises nucleotide sequence corresponding to the encoding sequence of CaMKII-δ9 or a portion thereof. Alternatively, the RNAi molecule is assembled from a single oligonucleotide, where the self-complementary sense and antisense regions of the RNAi molecule are linked by means of a nucleic acid-based or non-nucleic acid-based linker(s). The RNAi molecule can be a polynucleotide with a duplex, asymmetric duplex, hairpin or asymmetric hairpin secondary structure. The RNAi can be a circular single-stranded polynucleotide having two or more loop structures and a stem comprising self-complementary sense and antisense regions. The circular polynucleotide can be processed either in vivo or in vitro to generate an active RNAi molecule.

In some embodiments, the RNAi molecule has 10-100 bases, for example, may have about 10 to about 100 bases, about 15 to about 90 bases, about 20 to about 80 bases, about 25 to about 70 bases, about 30 to about 60 bases, about 35 to about 50 bases in length.

A small interfering RNA (siRNA) is a double-stranded RNA molecule that is capable of inhibiting or reducing the expression of a gene with which it shares homology. Each strand of the siRNA may have about 10 to about 100 bases, about 15 to about 90 bases, about 20 to about 80 bases, about 25 to about 70 bases, about 30 to about 60 bases, about 35 to about 50 bases in length. The double stranded siRNA may have about 10 to about 50 base pairs, about 12 to about 45 base pairs, about 15 to about 40 base pairs, about 20 to about 35 base pairs, about 20 to about 30 base pairs, or about 20 to about 25 base pairs.

A small hairpin RNA (shRNA) is an artificial RNA molecule with a tight hairpin turn that can be used to silence target gene expression via RNA interference. In some embodiments, the expression of shRNA in cells is accomplished by delivery of plasmids or through viral or bacterial vectors. A shRNA typically has about 10 to 100 base pairs in length, for example, about 10 to about 100 base pairs, about 15 to about 90 base pairs, about 20 to about 80 base pairs, about 25 to about 70 base pairs, about 30 to about 60 base pairs, or about 35 to about 50 base pairs in length.

A microRNA (miRNA) is a class of small noncoding RNAs which are involved in the regulation of gene expression at the post-transcriptional level by degrading their target mRNAs and/or inhibiting their translation. A miRNA typically has about 10 to 100 bases in length, for example, about 10 to about 100 bases, about 15 to about 90 bases, about 20 to about 80 bases, about 25 to about 70 bases, about 30 to about 60 bases, or about 35 to about 50 bases in length.

As used herein, the term “antisense nucleotide” refers to an oligomeric compound that is capable of undergoing hybridization to a target nucleic acid through hydrogen bonding. For example, “an antisense nucleotide that targets an encoding sequence of CaMKII-δ9” refers to a nucleotide that is capable of undergoing hybridization to the encoding sequence of CaMKII-δ9 or a portion thereof.

In some embodiments, the antisense nucleotide can be modified to improve its stability. Modifications to antisense nucleotides comprise substitutions or changes to internucleoside linkages, sugar moieties, or nucleobases. Modified antisense nucleotides are often preferred over native forms because of desirable properties such as enhanced cellular uptake, enhanced affinity for nucleic acid target, increased stability in the presence of nucleases, or increased inhibitory activity.

In some embodiments, the RNAi molecule or the antisense nucleotide is complementary to exon 16 of CaMKII-δ gene. In some embodiments, the RNAi molecule or the antisense nucleotide is complementary to exons 13-16 of CaMKII-5 gene. In some embodiments, the RNAi molecule or the antisense nucleotide is complementary to exons 16-17 of CaMKII-δ gene. In some embodiments, the RNAi molecule or the antisense nucleotide is complementary to exons 13-17 of CaMKII-5 gene. In some embodiments, the RNAi molecule or the antisense nucleotide is complementary to the encoding sequence of the full-length CaMKII-δ9.

As used herein, the term “complementary” or “complementarity” refers to the capacity for pairing between nucleobases of a first nucleic acid and a second nucleic acid. In some embodiments, the RNAi molecule or the antisense nucleotide provided herein are at least 50%, 60%, 70%, 80%, 85%, 90%, 91%, 92%, 93%, 94%, 95%, 96%, 97%, 98%, 99% or 100% complementary to a target nucleic acid, wherein the target nucleic acid is selected from the group consisting of exon 16 of CaMKII-δ gene, exons 13-16 of CaMKII-δ gene, exons 16-17 of CaMKII-δ gene, exons 13-17 of CaMKII-δ gene, and the encoding sequence of the full-length CaMKII-δ9. Percent complementarity of an RNAi molecule or an antisense nucleotide with a target nucleic acid can be determined using routine methods in the art.

In some embodiments, the antagonist is an agent that competes with CaMKII-δ9 to bind to its substrate.

As used herein, the term “compete” or “compete with . . . ” refers that the agent partially or completely inhibits the effect of CaMKII-δ9 by competing with it for binding to its substrate. The inhibition of CaMKII-δ9's binding to its substrate reduces or alters the normal level or type of cell signaling that occurs when CaMKII-δ9 binding to its substrate without such inhibition. Inhibition is also intended to include any measurable decrease in the binding of CaMKII-δ9 to its substrate when the antagonist as disclosed herein as compared to CaMKII-δ9 not in contact with the antagonist, e.g., by at least about 10%, 20%, 30%, 40%, 50%, 60%, 65%, 70%, 75%, 80%, 85%, 90%, 91%, 92%, 93%, 94%, 95%, 96%, 97%, 98%, 99%, or 100%.

In some embodiments, the agent that competes with CaMKII-δ9 to bind to its substrate is a vector that expresses CaMKII-δ9 which is without phosphorylation or oxidation function. In some embodiments, the vector of the present invention can be any gene transfer vector known in the art. In some embodiments, the vector of the present invention can comprise an exogenous gene comprising, consisting essentially of, or consisting of the encoding gene of CaMKII-δ9. In some embodiments, the CaMKII-δ9 expressed by the vector provided herein is without phosphorylation or oxidation function because the encoding gene that is responsible for the phosphorylation or oxidation function of CaMKII-δ9 is mutated, silenced or deleted. In some embodiments, the CaMKII-δ9 expressed by the vector provided herein is without phosphorylation or oxidation function because it undergoes post-translational processing so that its phosphorylation or oxidation function is eliminated. In some embodiments, the vector is an adeno-associated virus (AAV), an adenovirus, a lentivirus, a retrovirus, or a plasmid.

AAV is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV vector can be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316-327 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. In some embodiments, the AAV of the present invention is AAV1, AAV2, AAV5, AAV8, AAV9 or AAVrh10.

In another aspect, the present invention discloses methods of alleviating cardiac injury in a subject, comprising administering to the subject an effective amount of an antagonist of CaMKII-δ9. In yet another aspect, the present invention discloses use of an antagonist of CaMKII-δ9 in the manufacture of a medicament for alleviating cardiac injury in a subject. In yet another aspect, the present invention discloses an antagonist of CaMKII-δ9 for use in alleviating cardiac injury in a subject.

As used herein, the term “alleviate”, “alleviating” or “alleviation” include the decrease, limitation, or blockage of, for example, a particular action, function or interaction. In some embodiments, the cardiac injury is alleviated if at least one symptom of the cardiac injury is terminated, slowed or prevented. In some embodiments, the cardiac injury is alleviated if the level or activity of a protein that may cause cardiac injury (e.g. CaMKII-δ9) is decreased as compared to a reference state. Such alleviation can be partial or complete.

In another aspect, the present invention discloses methods of stimulating the level or activity of ubiquitin-conjugating enzyme in a subject, comprising administering to the subject an effective amount of an antagonist of CaMKII-δ9. In yet another aspect, the present invention discloses use of an antagonist of CaMKII-δ9 in the manufacture of a medicament for stimulating the level or activity of ubiquitin-conjugating enzyme in a subject. In yet another aspect, the present invention discloses an antagonist of CaMKII-δ9 for use in stimulating the level or activity of ubiquitin-conjugating enzyme in a subject. “The level or activity of ubiquitin-conjugating enzyme” refers to the amount of ubiquitin-conjugating enzyme in a subject, or the ability of ubiquitin-conjugating enzyme in a subject to target a protein for degradation via the proteasome during the ubiquitination reaction.

In some embodiments, the level or activity of ubiquitin-conjugating enzyme in a test biological sample is compared to a reference level or activity of ubiquitin-conjugating enzyme in a reference sample. The term “reference level or activity” as used herein refers to a threshold level or activity of a substance in a subject. For example, if the level or activity of ubiquitin-conjugating enzyme of a test biological sample which is from a subject received an antagonist of CaMKII-δ9 is higher than the reference level or activity of ubiquitin-conjugating enzyme in a reference sample, then the antagonist of CaMKII-δ9 may be considered as stimulating the level or activity of ubiquitin-conjugating enzyme in the subject. A reference level or activity of ubiquitin-conjugating enzyme may be derived from one or more reference samples wherein the reference level or activity is obtained from experiments conducted in parallel with the experiment for testing the sample of interest. Alternatively, a reference level or activity may be obtained in a reference database, which includes a collection of data, standard, level or activity from one or more reference samples or disease reference samples. In some embodiments, such collection of data, standard, level or activity are normalized so that they can be used for comparison purpose with data from one or more samples. “Normalize” or “normalization” is a process by which a measurement raw data is converted into data that may be directly compared with other so normalized data. Normalization is used to overcome assay-specific errors caused by factors that may vary from one assay to another, for example, variation in loaded quantities, binding efficiency, detection sensitivity, and other various errors. In certain embodiment, a reference database includes concentrations of ubiquitin-conjugating enzyme and/or other laboratory and clinical data from one or more reference samples. In some embodiments, a reference database includes levels or activity of ubiquitin-conjugating enzyme that are each normalized as a percent of the level or activity of ubiquitin-conjugating enzyme of a reference sample (e.g. a known amount or activity of ubiquitin-conjugating enzyme) tested under the same conditions as the reference samples. In order to compare with such normalized levels or activities of ubiquitin-conjugating enzyme, the level or activity of ubiquitin-conjugating enzyme of a test biological sample is also measured and calculated as a percent of the level or activity of ubiquitin-conjugating enzyme of the reference sample tested under the same conditions as the test sample.

Without being bound to any theory, but it is contemplated that an increased level or activity of ubiquitin-conjugating enzyme in a subject is beneficial to the subject. In some embodiments, the level or activity of ubiquitin-conjugating enzyme detected in the test biological sample is at least 2 times the reference level or activity of ubiquitin-conjugating enzyme. In some embodiments, the level or activity of ubiquitin-conjugating enzyme detected in the test biological sample is at least 3, 4, 5, 6, 7, 8, 9, 10, 15, 20, 25, 30 times the reference level or activity of ubiquitin-conjugating enzyme.

In some embodiments, the reference sample is from a healthy subject or is a sample obtained from the same subject earlier or later than the test biological sample.

As used herein, the term “healthy subject” refers to a subject who is known, or believed, not to be afflicted with the disease, condition or disorder for which a method or composition of the present invention is being used to identify. In some embodiments, the reference sample is obtained from a healthy part of the body of the same subject in whom a disease or condition is being identified using a method or composition of the present invention. In some embodiments, the test biological sample is from the heart of the subject. In some embodiments, the subject is a human or non-human primate.

In another aspect, the present invention discloses methods of preventing degradation of ubiquitin-conjugating enzyme in a subject, comprising administering to the subject an effective amount of an antagonist of CaMKII-δ9. In yet another aspect, the present invention discloses use of an antagonist of CaMKII-δ9 in the manufacture of a medicament for preventing degradation of ubiquitin-conjugating enzyme in a subject. In yet another aspect, the present invention discloses an antagonist of CaMKII-δ9 for use in preventing degradation of ubiquitin-conjugating enzyme in a subject.

As used herein, “the degradation of ubiquitin-conjugating enzyme” refers that the level or activity of ubiquitin-conjugating enzyme in a test biological sample is decreased compared to a reference level or activity of ubiquitin-conjugating enzyme in a reference sample. Without being bound to any theory, but it is contemplated that the degradation of ubiquitin-conjugating enzyme in a subject is harmful to the subject. In some embodiments, the antagonist of CaMKII-δ9 disclosed herein can prevent, for example, at least 10%, 20%, 30%, 40%, 50%, 60%, 70%, 80%, 85%, 90%, 91%, 92%, 93%, 94%, 95%, 96%, 97%, 98%, 99% or 100% of the amount or activity of ubiquitin-conjugating enzyme in a test biological sample from degradation.

In another aspect, the present invention discloses methods of preventing cardiomyocyte death in a sample, comprising contacting the sample with an effective amount of an antagonist of CaMKII-δ9. In yet another aspect, the present invention discloses use of an antagonist of CaMKII-δ9 in the manufacture of a medicament for preventing cardiomyocyte death in a sample. In yet another aspect, the present invention discloses an antagonist of CaMKII-δ9 for use in preventing cardiomyocyte death in a sample. In some embodiments, the method or use provided herein can prevent, for example, at least 10%, 20%, 30%, 40%, 50%, 60%, 70%, 80%, 85%, 90%, 91%, 92%, 93%, 94%, 95%, 96%, 97%, 98%, 99%, or 100% cardiomyocyte in a test sample from death.

In another aspect, the present invention discloses methods of reducing DNA damage in a cell, comprising contacting the cell with an effective amount of an antagonist of CaMKII-δ9. In yet another aspect, the present invention discloses use of an antagonist of CaMKII-δ9 in the manufacture of a medicament for reducing DNA damage in a cell. In yet another aspect, the present invention discloses an antagonist of CaMKII-δ9 for use in reducing DNA damage in a cell. As used herein, the term “DNA damage” refers to alteration in the chemical structure of DNA, such as a break in a strand of DNA, a base missing from the backbone of DNA, or a chemically changed base.

Cells are continually exposed to factors, such as intracellular reactive species and environmental agents, which are capable of causing DNA damage. The potentially mutagenic consequences of DNA damage are minimized by DNA repair pathways, which are broadly characterized into three forms: base excision repair (BER), mismatch repair (MMR), and nucleotide excision repair (NER) (Wood et al., Science, 291: 1284-1289 (2001)). In some embodiments, the antagonists of the present invention may activate the DNA repair pathways by, for example, inhibit the level and/or activity of CaMKII-δ9 in the cell.

In another aspect, the present invention discloses methods for diagnosing a CaMKII-mediated disease in a subject comprising: (a) obtaining a test biological sample of the subject, and (b) detecting a level or activity of CaMKII-δ9 in the test biological sample; wherein the level or activity of CaMKII-δ9 detected in the test biological sample of the subject is indicative of the subject developing or with an increased probability of developing a CaMKII-mediated disease.

In yet another aspect, the present invention discloses use of an agent in the manufacture of a medicament for diagnosing a CaMKII-mediated disease in a subject, wherein the diagnosing comprises (a) obtaining a test biological sample of the subject, and (b) detecting a level or activity of CaMKII-δ9 in the test biological sample; wherein the level or activity of CaMKII-δ9 detected in the test biological sample of the subject is indicative of the subject developing or with an increased probability of developing a CaMKII-mediated disease.

In yet another aspect, the present invention discloses an agent for use in diagnosing a CaMKII-mediated disease in a subject, wherein the diagnosing comprises (a) obtaining a test biological sample of the subject, and (b) detecting a level or activity of CaMKII-δ9 in the test biological sample; wherein the level or activity of CaMKII-δ9 detected in the test biological sample of the subject is indicative of the subject developing or with an increased probability of developing a CaMKII-mediated disease.

As used herein, the term “diagnosis” or “diagnosing” refers to the identification of a pathological state, disease or condition, such as identification of a CaMKII-mediated disease, or refer to identification of a subject with a CaMKII-mediated disease who may benefit from a particular treatment regimen. In some embodiments, diagnosis contains the identification of abnormal level or activity of CaMKII-δ9. In some embodiments, diagnosis refers to the identification of a heart disease or a metabolic disease in a subject.

As used herein, the term “biological sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject. In some embodiments, the biological sample is obtained from the heart of the subject.

In accordance with the present invention, detecting the level or activity of a peptide of interest (e.g. ubiquitin-conjugating enzyme, CaMKII-δ9, etc.) in the test biological sample can be achieved by any suitable means for determining the level or activity of a peptide in a sample. In some embodiments, the detection methods include immunoassay devices and methods which may utilize labeled molecules in various sandwich, competition, or other assay formats. Said assays will develop a signal which is indicative of the presence or absence of the peptide of interest. Moreover, the signal strength can be correlated directly or indirectly (e.g., reverse-proportional) to the amount of the peptide of interest present in a sample. Further suitable methods include measuring a physical or chemical property specific for the peptide of interest such as its precise molecular mass or NMR spectrum. Suitable detection methods may also include biosensors, optical devices coupled to immunoassays, biochips, analytical devices such as mass-spectrometers, NMR-analyzers, or chromatography devices. Further, suitable detection methods may include micro-plate ELISA-based methods, fully-automated or robotic immunoassays (available for example on ELECSYS analyzers), CBA (an enzymatic Cobalt Binding Assay, available for example on Roche-Hitachi analyzers), and latex agglutination assays (available for example on Roche-Hitachi analyzers). In some embodiments, the level or activity of a peptide of interest is detected by measuring a specific intensity signal obtainable from the peptide in the sample. As described above, such a signal may be the signal intensity observed at an m/z (mass to charge ratio) variable specific for the peptide of interest observed in mass spectra or a NMR spectrum specific for the peptide of interest.

In some embodiments, the level or activity of CaMKII-δ9 in the test biological sample is detected by contacting the sample with a reagent that specifically binds to CaMKII-δ9. The reagents will generate intensity signals. Binding according to the present invention includes both covalent and non-covalent binding. A reagent binding to CaMKII-δ9 according to the present invention can be any compound, e.g., a peptide, polypeptide, nucleic acid, or small molecule, binding to CaMKII-δ9 described herein. In some embodiments, the reagents include antibodies, nucleic acids, peptides or polypeptides such as receptors or binding partners for the peptide or fragments thereof containing the binding domains for the peptides, and aptamers, e.g., nucleic acid or peptide aptamers. Methods to prepare such reagents are well-known in the art. For example, identification and production of suitable antibodies or aptamers is also offered by commercial suppliers. Those of skill in the art are familiar with methods to develop derivatives of such reagents with higher affinity or specificity. For example, random mutations can be introduced into the nucleic acids, peptides or polypeptides. These derivatives can then be tested for binding according to screening procedures known in the art, e.g., phage display.

In some embodiments, non-specific binding may be tolerable, if it can still be distinguished and measured unequivocally, e.g., according to its size on a Western Blot, or by its relatively higher abundance in the sample. Binding of the reagent can be measured by any method known in the art. In some embodiments, said method is semi-quantitative or quantitative. Suitable methods include: (1) binding of a reagent may be measured directly, e.g., by NMR or surface plasmon resonance; (2) if the reagent also serves as a substrate of an enzymatic activity of the peptide of interest, an enzymatic reaction product may be measured (e.g., the amount of a protease can be measured by measuring the amount of cleaved substrate, e.g., on a Western Blot). Alternatively, the reagent may exhibit enzymatic properties itself and the reagent which was bound by the peptide, may be contacted with a suitable substrate allowing detection by the generation of an intensity signal. For measurement of enzymatic reaction products, in some embodiments, the amount of substrate is saturating. The substrate may also be labeled with a detectable label prior to the reaction. In some embodiments, the sample is contacted with the substrate for an adequate period of time. An adequate period of time refers to the time necessary for a detectable or measurable, amount of product to be produced. Instead of measuring the amount of product, the time necessary for appearance of a given (e.g., detectable) amount of product can be measured; (3) the reagent may be coupled covalently or non-covalently to a label allowing detection and measurement of the reagent. Labeling may be done by direct or indirect methods. Direct labeling involves coupling of the label directly (covalently or non-covalently) to the reagent. Indirect labeling involves binding (covalently or non-covalently) of a secondary reagent to the first reagent. The secondary reagent should specifically bind to the first reagent. Said secondary reagent may be coupled with a suitable label and/or be the target (receptor) of tertiary reagent binding to the secondary reagent. The use of secondary, tertiary or even higher order reagents is often to increase the signal intensity. Suitable secondary and higher order reagents may include antibodies, secondary antibodies, and the well-known streptavidin-biotin system (Vector Laboratories, Inc.). The reagent or substrate may also be “tagged” with one or more tags as known in the art. Such tags may then be targets for higher order reagents. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is at the N-terminus and/or C-terminus.

In some embodiments, the reagent that specifically binds to CaMKII-δ9 is an antibody or an antibody fragment. In some embodiments, the antibody is a monoclonal antibody.

In some embodiments, the level or activity of CaMKII-δ9 detected in the test biological sample is compared to a reference level or activity of CaMKII-α9 detected in a reference sample.

As used herein, the term “compare” refers to comparing the level or activity of a target protein (e.g. ubiquitin-conjugating enzyme, CaMKII-δ9, etc.) comprised by the test biological sample to be analyzed with a level or activity of a suitable reference sample. It is to be understood that the term as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison may be carried out manually or computer assisted. For a computer assisted comparison, the value of the determined amount may be compared to values corresponding to suitable references which are stored in a database by a computer program. The computer program may further evaluate the result of the comparison, and automatically provide the desired assessment in a suitable output format. Based on the comparison of the level or activity of CaMKII-δ9 detected to suitable reference level(s), it is possible to diagnose CaMKII-mediated diseases in said subject.

In some embodiments, a higher level or activity of CaMKII-δ9 detected in the test biological sample than the reference level or activity of CaMKII-δ9 is indicative of the subject developing or with an increased probability of developing a CaMKII-mediated disease. Preferably, the level or activity of CaMKII-δ9 detected in the test biological sample is at least 2 times the reference level or activity of CaMKII-δ9. More preferably, the level or activity of CaMKII-δ9 detected in the test biological sample is at least 3, 4, 5, 6, 7, 8, 9, 10, 15, 20, 25, 30 times the reference level or activity of CaMKII-δ9.

As used herein, the term “increased probability” refers to an overall increase of 5%, 10%, 15%, 20%, 25%, 30%, 40%, 50%, 60%, 70%, 80%, 85%, 90%, 95%, 96%, 97%, 98%, 99%, one time, two times, three times, five times, eight times, ten times, twenty times, fifty times or greater, in the level of likelihood that a subject will develop CaMKII-mediated diseases, as compared to a subject from which a reference sample is obtained.

In some embodiments, the reference sample is from a healthy subject or is a sample obtained from the same subject earlier or later than the test biological sample.

In yet another aspect, the present invention discloses a kit for diagnosing a CaMKII-mediated disease in a subject, comprising an antibody or an antibody fragment that specifically recognizes CaMKII-δ9. In some embodiments, the antibody or the antibody fragment specifically binds to an amino acid sequence encoded by exon 16 of CaMKII-δ gene. The kit may use any means suitable for detecting the content or the activity of CaMKII-δ9 in a sample.

In some embodiments, the kit may additionally contain a user's manual for interpreting the results of any measurement(s) with respect to diagnose a CaMKII-mediated disease in a subject as defined in the present application. Particularly, such manual may include information about what determined levels corresponds to what kind of diagnosis. Additionally, such user's manual may provide instructions about correctly using the components of the kit for detecting the level of CaMKII-δ9. In some embodiments, the means for detection and the instruction manual of the kit are provided within a single container.

In one aspect, the present invention discloses a method for identifying a molecule that inhibits the activities of CaMKII-δ9, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that inhibits CaMKII-δ9.

In another aspect, the present invention discloses a method for identifying a molecule that inhibits the phosphorylation capability of CaMKII-δ9, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that inhibits the phosphorylation capability of CaMKII-δ9.

In yet another aspect, the present invention discloses a method for identifying a molecule that treats or prevents a CaMKII-mediated disease, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that treats or prevents a CaMKII-mediated disease.

In yet another aspect, the present invention discloses a method for identifying a molecule that alleviates cardiac injury, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that alleviates cardiac injury.

In yet another aspect, the present invention discloses a method for identifying a molecule that prevents cardiomyocyte death, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that prevents cardiomyocyte death.

In yet another aspect, the present invention discloses a method for identifying a molecule that reduces DNA damage, comprising contacting the molecule with a sample comprising (i) CaMKII-δ9 and (ii) UBE2T, and determining whether the phosphorylation of UBE2T is inhibited, wherein the inhibition of the phosphorylation of UBE2T identifies a molecule that reduces DNA damage.

These methods are also referred to herein as drug screening assays and typically include the step of screening a candidate/test molecule for the ability to interact with (e.g., bind to) CaMKII-δ9, to modulate the phosphorylation of UBE2T by CaMKII-δ9, and/or to modulate the interaction of a phosphorylatable residue of UBE2T with a CaMKII-δ9-mediated intracellular signaling target.

In some embodiments, the method further comprises a step of determining whether the molecule directly binds said CaMKII-δ9.

In some embodiments, the inhibition of the phosphorylation of UBE2T is determined by comparing the amount of phosphorylated UBE2T, in the sample relative to a control. In some embodiments, the control is the amount of phosphorylated UBE2T in the sample relative to said amount in the absence of the molecule or at an earlier timepoint after contact of the sample with the molecule. In some embodiments, the inhibition of the phosphorylation of UBE2T is determined by comparing the ratio of the amount of the phosphorylation of UBE2T, in the sample relative to the total amount of UBE2T, to a control. In some embodiments, the control is the ratio of the amount of phosphorylated UBE2T in the sample relative to said ratio in the absence of the molecule or at an earlier timepoint after contact of the sample with the molecule.

In some embodiments, the sample is selected from the group consisting of in vitro, ex vivo, and in vivo samples. In some embodiments, the sample comprises cells (e.g. heart cells). In some embodiments, the cells are obtained from a subject. In some embodiments, the sample is selected from the group consisting of tissue, whole blood, serum, plasma, buccal scrape, saliva, cerebrospinal fluid, urine, stool, and bone marrow.

In some embodiments, the candidate/test molecule used in the drug screening assay is a small molecule compound, or an antibody or antigen-binding fragment thereof. In some embodiments, the candidate/test molecule decreases the amount of phosphorylated UBE2T by at least 50%, 60%, 70%, 80%, 90%, 95%, 98% or 99%.

The candidate/test molecules of the present invention to be identified, at least in part, can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In some embodiments, the phosphorylation of UBE2T is at any Serine (Ser), Threonine (Thr), or Tyrosine (Tyr) position. In some embodiments, the phosphorylation of UBE2T is at Ser5, Ser81, Ser82, Ser101, Ser110, Ser129, Ser130, Ser165, Ser166, Ser172, Ser174, Ser176, Ser177, Ser193 or Ser204. In some embodiments, the phosphorylation of UBE2T is at Ser110. In some embodiments, the phosphorylation of UBE2T is at Thr23, Thr44, Thr52, Thr72, Thr106, Thr109, Thr147, Thr177 or Thr178. In some embodiments, the phosphorylation of UBE2T is at Tyr46, Tyr61, Tyr74 or Tyr134. The positions of amino acids mentioned herein refer to the positions of Serine, Threonine and Tyrosine in wild-type UBE2T, for example, of human, mouse and rat.

In one aspect, the present invention discloses an isolated CaMKII-δ polypeptide comprising an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5. In some embodiments, the isolated CaMKII-δ polypeptide is not a full-length natural polypeptide. In some embodiments, the length of the isolated CaMKII-δ polypeptide is 14 amino acids, 27 amino acids, 30 amino acids, 43 amino acids, or 513 amino acids.

In some embodiments, the present invention discloses an isolated CaMKII-δ polypeptide having an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5.

As used herein, the term “isolated” refers that a substance (such as a polypeptide or a nucleic acid) is separated from the environment in which it is normally present in nature or in an environment different from the environment in which it is normally found in nature.

As used herein, the percent (%) “sequence homology to” refers to, for amino acid sequences, the percentage of identity between two amino acid sequences after aligning the candidate and the reference sequences, and if necessary introducing gaps, to achieve the maximum number of identical amino acids; for nucleotide sequence, the percentage of identity between two nucleotide sequences after aligning the candidate and the reference sequences, and if necessary introducing gaps, to achieve the maximum number of identical nucleotides.

The percentage of homology can be determined by various well-known methods in the art. For example, the comparison of sequences can be achieved by the following publically available tools: BLASTp software (available from the website of National Center for Biotechnology Information (NCBI): http://blast.ncbi.nlm.nih.gov/Blast.cgi, also see, Altschul S F et al., J. Mol. Biol., 215: 403-410 (1990); Stephen F. et al., Nucleic Acids Res., 25: 3389-3402 (1997)), ClustalW2 (available from the website of European Bioinformatics Institute: http://www.ebi.ac.uk/Tools/msa/clustalw2/, see Higgins D G et al., Methods in Enzymology, 266: 383-402 (1996); Larkin M A et al., Bioinformatics (Oxford, England), 23 (21): 2947-8 (2007)), and TCoffee (available from the Swiss Institute of Bioinformatics website, also see Poirot O. et al., Nucleic Acids Res., 31 (13): 3503-6 (2003); Notredame C. et al., J. Mol. Boil., 302 (1): 205-17 (2000)). If the alignment of the sequences is performed using software, the default parameters available in the software may be used, or otherwise the parameters may be customized to suit the alignment purpose. All of these are within the scope of knowledge of those of skill in the art.

Conservative substitutions of amino acid residues refer to substitutions between amino acids with similar characteristics, such as substitutions between polar amino acids (e.g. substitutions between glutamine and asparagine), substitutions between hydrophobic amino acids (e.g. substitution among arginine, isoleucine, methionine and valine), and substitutions between amino acids with the same charge (e.g. substitutions among arginine, lysine and histidine, or between glutamine and aspartate), etc.

In another aspect, the present invention discloses an isolated CaMKII-δ nucleic acid comprising a nucleic acid sequence encoding the CaMKII-δ polypeptide comprising an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5.

As used herein, the term “nucleic acid” or “polynucleotide” refers to ribonucleic acid (RNA), deoxyribonucleic acid (DNA), or a mixture of ribonucleoside-deoxyribonucleic acids such as DNA-RNA hybrids. The nucleic acids or polynucleotides can be single- or double-stranded DNA, RNA, or DNA-RNA hybrids. Nucleic acids or polynucleotides may be linear or cyclic.

In some embodiments, the CaMKII-δ nucleic acid comprises one of the nucleic acid sequences selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and a nucleic acid sequence having at least 80% homology to SEQ ID NOs: 6-19.

In some embodiments, the isolated CaMKII-δ nucleic acids provided herein comprise the nucleic acid sequence that has at least 70% homology, e.g., at least 75%, at least 80%, at least 85%, at least 90%, at least 95%, or at least 99% homology to any one of the nucleic acid sequences set forth in SEQ ID NOs: 6-19, and can still encode the CaMKII-δ polypeptide comprising an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5.

In some embodiments, the present invention provides CaMKII-δ nucleic acid sequences encoding the CaMKII-δ polypeptide comprising an amino acid sequence set forth in SEQ ID NO: 1, an amino acid sequence set forth in SEQ ID NO: 2, an amino acid sequence set forth in SEQ ID NO: 3, an amino acid sequence set forth in SEQ ID NO: 4, an amino acid sequence set forth in SEQ ID NO: 5, or an amino acid sequence having at least 80% homology to the amino acid sequences set forth in SEQ ID NOs: 1-5, but they are different from any one of the nucleic acid sequences set forth in SEQ ID NOs: 6-19 due to the degeneracy of the genetic code.

In another aspect, the present invention discloses a CaMKII antagonist capable of inhibiting the activity of CaMKII-δ9.

In some embodiments, the antagonist is an antagonist for inhibiting the phosphorylation of ubiquitin-conjugating enzyme by CaMKII-δ9. In some embodiments, the ubiquitin-conjugating enzyme is UBE2T. In some embodiments, the antagonist is an antagonist for inhibiting the phosphorylation of UBE2T at Ser110.

In some embodiments, the antagonist is an antibody that binds to the amino acid sequence encoded by exon 16 of CaMKII-δ gene, an RNAi molecule that targets exon 16 of CaMKII-δ gene, an antisense nucleotide that targets exon 16 of CaMKII-δ gene.

In another aspect, the present invention discloses a pharmaceutical composition comprising the antagonist capable of inhibiting the activity of CaMKII-δ9 as disclosed in the present invention.

In some embodiments, the pharmaceutical composition further comprises a pharmaceutically acceptable carrier.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments, compounds, materials, compositions, and/or dosage forms that are pharmaceutically acceptable refer to those approved by a regulatory agency (such as U.S. Food and Drug Administration, China Food and Drug Administration or European Medicines Agency) or listed in generally recognized pharmacopoeia (such as U.S. Pharmacopoeia, China Pharmacopoeia or European Pharmacopoeia) for use in animals, and more particularly in humans.

The pharmaceutically acceptable carriers for use in the pharmaceutical compositions of the present invention may include, but are not limited to, for example, pharmaceutically acceptable liquids, gels, or solid carriers, aqueous vehicles (e.g., sodium chloride injection, Ringer's injection, isotonic glucose injection, sterile water injection, or Ringer's injection of glucose and lactate), non-aqueous vehicles (e.g., fixed oils of vegetable origin, cottonseed oil, corn oil, sesame oil, or peanut oil), antimicrobial agents, isotonic agents (such as sodium chloride or dextrose), buffers (such as phosphate or citrate buffers), antioxidants (such as sodium bisulfate), anesthetics (such as procaine hydrochloride), suspending/dispending agents (such as sodium carboxymethylcellulose, hydroxypropyl methylcellulose, or polyvinylpyrrolidone), chelating agents (such as EDTA (ethylenediamine tetraacetic acid) or EGTA (ethylene glycol tetraacetic acid)), emulsifying agents (such as Polysorbate 80 (TWEEN-80)), diluents, adjuvants, excipients, or non-toxic auxiliary substances, other components known in the art, or various combinations thereof. Suitable components may include, for example, fillers, binders, disintegrants, buffers, preservatives, lubricants, flavorings, thickeners, coloring agents, or emulsifiers.

In some embodiments, the pharmaceutical composition is an oral formulation. The oral formulations include, but are not limited to, capsules, cachets, pills, tablets, troches (for taste substrates, usually sucrose and acacia or tragacanth), powders, granules, or aqueous or non-aqueous solutions or suspensions, or water-in-oil or oil-in-water emulsions, or elixirs or syrups, or confectionery lozenges (for inert bases, such as gelatin and glycerin, or sucrose or acacia) and/or mouthwash and its analogs.

In some embodiments, the oral solid formulation (e.g., capsules, tablets, pills, dragees, powders, granules, etc.) includes the active substance and one or more pharmaceutically acceptable carriers, such as sodium citrate or dicalcium phosphate, and/or the followings: (1) fillers or extenders such as starch, lactose, sucrose, glucose, mannitol and/or silicic acid; (2) binders such as, carboxymethylcellulose, alginates, gelatin, polyvinylpyrrolidone, sucrose, and/or acacia; (3) humectants such as glycerol; (4) cleaving agents such as agar, calcium carbonate, potato or tapioca starch, alginic acid, certain silicates, and sodium carbonate; (5) retarder solutions such as paraffin; (6) accelerating absorbers such as quaternary ammonium compounds; (7) lubricants such as acetyl alcohol and glycerol monostearate; (8) absorbents such as kaolin and bentonite; (9) lubricants such as talc, calcium stearate, magnesium stearate, solid polyethylene glycol, sodium sulfate, and mixtures thereof; and (10) colorants.

In some embodiments, the oral liquid formulation includes pharmaceutically acceptable emulsions, microemulsions, solutions, suspensions, syrups and elixirs, etc. In addition to the active substance, the liquid dosage forms may also contain conventional inert diluents such as water or other solvents, solubilizers and emulsifiers such as ethanol, isopropanol, ethyl carbonate, ethyl acetate, benzyl alcohol, benzene (meth) acrylate, propylene glycol, 1,3-butylene glycol, oils (in particular, cottonseed, groundnut, corn, olive, castor and sesame oils), glycerol, tetrahydrofurfuryl alcohol, polyethylene glycol and fatty acid sorbitol esters, and mixtures thereof. Besides inert diluents, the oral compositions may also contain adjuvants such as wetting agents, emulsifying and suspending agents, sweetening, flavoring, coloring, flavoring and preserving agents.

In some embodiments, the pharmaceutical composition may be an injectable formulation, including sterile aqueous solutions or dispersions, suspensions or emulsions. In all cases, the injectable formulation should be sterile and should be liquid to facilitate injections. It should be stable under the conditions of manufacture and storage, and should be resistant to the infection of microorganisms (such as bacteria and fungi). The carrier may be a solvent or dispersion medium containing, for example, water, ethanol, polyols (e.g., glycerol, propylene glycol, and liquid polyethylene glycols, etc.) and suitable mixtures and/or vegetable oils thereof. The injectable formulation should maintain proper fluidity, which may be maintained in a variety of ways, for example, using a coating such as lecithin, using a surfactant, etc. Antimicrobial contamination can be achieved by the addition of various antibacterial and antifungal agents (e.g., parabens, chlorobutanol, phenol, sorbic acid, thimerosal, etc.).

In some embodiments, the pharmaceutical composition is an oral spray formulation or nasal spray formulation. Such spray formulations include, but are not limited to, aqueous aerosols, non-aqueous suspensions, liposomal formulations, or solid particulate formulations, etc. Aqueous aerosols are formulated by combining an aqueous solution or suspension of the agent with a conventional pharmaceutically acceptable carrier and stabilizer. The carrier and stabilizer may vary according to the needs of specific compounds, but generally include nonionic surfactants (Tweens, or polyethylene glycol), oleic acid, lecithin, amino acids such as glycine, buffers, salts, sugar or sugar alcohol. Aerosols are usually prepared from isotonic solutions and can be delivered by nebulizers.

In some embodiments, the pharmaceutical compositions may be used in combination with one or more other drugs. In some embodiments, the composition comprises at least one other drug. In some embodiments, the other drugs are cardiovascular drugs, drugs for treating kidney diseases, drugs for cell membrane repair, etc.

In some embodiments, the pharmaceutical compositions may be delivered to the subject by suitable routes including, but not limited to, the oral route, injection route (e.g., intravenous injection, intramuscular injection, subcutaneous injection, intradermal injection, intracardiac injection, intrathecal injection, intrapleural injection, intraperitoneal injection, etc.), mucosal route (e.g., intranasal administration, oral administration, etc.), sublingual route, rectal route, transdermal route, intraocular route, pulmonary route. In some embodiments, the pharmaceutical compositions can be administered by injection route.

### Embodiments

The biological materials used in all examples, various clones and expression plasmids, media, enzymes, buffer solutions, and various culturing methods, protein extraction and purification methods, and the other molecular biological operation methods, are all well-known to those of skill in the art. For more details, please refer to the “Molecular Cloning: A Laboratory Manual” edited by Sambrook, et al. (Cold Spring Harbor, 1989) and “Short Protocols in Molecular Biology” (Frederick M. Ausubel, et al., translated by Yan Ziying et al., Science Press (Beijing), 1998).

General Methods and Materials

1.1 Animals

Animals were maintained in the Center for Experimental Animals (an Association for Assessment and Accreditation of Laboratory Animal Care-accredited experimental animal facility) at Peking University, Beijing, China. The animals were randomly allocated to experimental groups. Only males were used. No non-inclusion or exclusion parameters were used in our studies. Investigators were not blinded to treatments, but no subjective assessments were made. All procedures involving experimental animals (mice, rats, and rhesus monkeys) followed protocols approved by the Committee for Animal Research of Peking University and conformed to the Guide for the Care and Use of Laboratory Animals.

Adult C57BL/6 mice and Sprague-Dawley rats were from Vital River Laboratories, Beijing, China. Rhesus monkeys were from our in-house cohort as previously reported (Zhang, X. et al., Circulation 124, 77-86, doi:10.1161/CIRCULATIONAHA.110.990333 (2011)). The animals were euthanized by intravenous injection of an overdose of sodium pentobarbital, and the tissues were quickly frozen in liquid nitrogen for protein and total RNA extraction.

1.2 Animal Surgery and Treatment

Transverse aortic constriction (TAC) was performed in 6-week-old male mice. Mice were anesthetized under 3% isoflurane via intubation, the chest was opened, the aortic arch was visualized, and a 7-0 silk suture was passed under the arch between the innominate and left common carotid arteries. The suture was secured around both the aorta and a 28-gauge needle, the needle was removed, the chest was closed, and the mouse was extubated. Sham-operated mice underwent an identical procedure except for the aortic ligation. Mice were given buprenorphine via intraperitoneal injection during recovery.

1.3 Library Preparation, Sequencing, and Data Collection for SMRT Sequencing

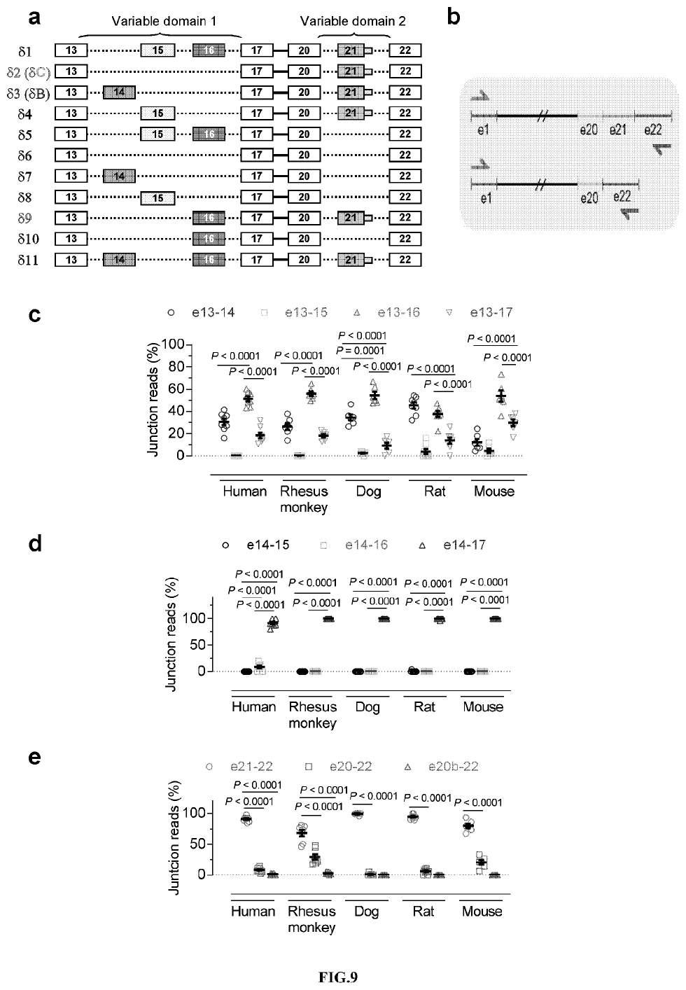

Total RNA from the left ventricle of normal mice, rats, and rhesus monkeys was prepared using the RNeasy Fibrous Tissue Mini Kit (Qiagen, Cat #: 74704). Total RNA from the left ventricle of normal adult humans were from MY Biosource (Cat #: MBS537570), Biological (Cat #: T5595-7325), and Biochain (Cat #: R1234138-50). Two micrograms of total RNA were used for first-strand synthesis. CaMKII-δ-specific primers that anneal in the first and the last coding exon were used for cDNA amplification (FIG. 9b). PCR products corresponding to full-length CaMKII-δ (˜1600 bp) were concentrated using a PCR production purification kit (Qiagen, Cat #: 28004). SMRTbell sequencing libraries were prepared following the standard PacBio guidelines. Sequencing was carried out on a Pacific Biosciences real-time sequencer using C2 sequencing reagents in the Wuhan Institute of Biotechnology Public Technology Service Platform. Subread filtering was performed using the SMRT analysis software (v2.2) of Pacific Biosciences. Circular consensus (CCS) reads in FASTQ format were mapped to CaMKII-δ gene loci from all sample species, using GMAP (version 2014-09-29) with the following parameters: - -format=1 -batch=2 -nthreads=6 -trimendexons=4 - -ordered. Meanwhile, primers/barcodes were detected using flexbar (version 2.5) with the following parameters: - -threads 6 - -barcode-min-overlap 8 - -barcode-threshold 2 - -log-level TAB - -barcode-keep - -barcode-unassigned. Alignments were kept only if they came from the same species as the best mapping and primer indicated. Subsequent analysis referred to Treutlein et al. PNAS 111, E1291-1299, doi:10.1073/pnas.1403244111 (2014). Alignments were parsed to splice junctions (i.e. variant structure) with 3-bp gap tolerance. Only CCS reads parsed to unambiguous and accordant splice junctions were kept. Then the frequencies of splice junctions were calculated.

The primers were as follows:

| Species | Direction | Sequence 5′-3′ |
|---|---|---|
| Human | Forward | GCCAGGGCACAGCCCGGACCG (SEQ ID NO: 20) |
| Human | Reverse | GAGAATGCAGAAGTGGCACTG (SEQ ID NO: 21) |
| Rhesus monkey | Forward | GGGCACAGCCCGGACCAAGG (SEQ ID NO: 22) |
| Rhesus monkey | Reverse | ATGCAGAAGTGGCACTGTTG (SEQ ID NO: 23) |
| Rat | Forward | GGGCACAGCCCGGACCAAGG (SEQ ID NO: 24) |
| Rat | Reverse | ATGCAGAAGTGGCACTGTTG (SEQ ID NO: 25) |
| Mouse | Forward | GGCGAGCTACTTTCGGACAC (SEQ ID NO: 26) |
| Mouse | Reverse | GAAGAAGTGGCACTGTTGAC (SEQ ID NO: 27) |

1.4 Human Samples

Human ventricular tissue from patients with hypertrophic cardiomyopathy was obtained during hypertrophic ventricular septum myectomy surgery in Fuwai Hospital, Beijing, China, and the protocol was approved by the Ethics Committee of Fuwai Hospital, the Chinese Academy of Medical Sciences, and Peking Union Medical College. Our study is compliant with all the ethical regulations. All patients provided written informed consent. Normal human ventricular tissue was from the NIH NeuroBioBank at the University of Maryland, Baltimore, Md.

1.5 RNA-Sea (2nd-Generation Sequencing) and CaMKII-δ Exon Junction Analysis

Total RNA of mouse tissue was prepared using the RNeasy Mini Kit (Qiagen Cat #: 74104), and the sequencing process was as reported previously (Liu, F. et al. Circulation 131, 795-804, doi:10.1161/CIRCULATIONAHA.114.012285 (2015)). The RNA-seq data for other species were from the National Center for Biotechnology Information (NCBI) Sequence Read Archive database as follows: human left ventricle (SRR830965, SRR830966, SRR830967, SRR830968, SRR830969, SRR830970, SRR830971, and SRR830972), rhesus monkey heart (SRX196319, SRX196328, SRX196337, SRX081927, SRX081928, SRX494639, and SRX066573), dog left ventricle (SRR1735880, SRR1735881, SRR1735882, SRR1735883, SRR1735884, and SRR1735885), and rat heart (SRX471444, SRX471445, SRX471446, SRX471447, SRX471460, SRX471461, SRX471462, and SRX471463). The exon structure of CaMKII-δ splice variants shown in FIG. 9a was summarized from the RefSeq, Uniprot, and UCSC KnownGene databases. The classification of CaMKII-δ refers to Mayer (Mayer, P. et al., The Biochemical journal 298 Pt 3, 757-758 (1994)). The structure for mouse CaMKII-5 was retrieved using LiftOver with default parameters. By multiple sequence alignment, the inventors found two main alternative splicing domains, one between exons 13 and 17 and the other between exons 20 and 22. The RNA-seq reads were mapped to the mouse (mm9), rat (rn4), dog (canFam2), rhesus (rheMac2), or human genome (hg19) using TopHat-2.0.8 with default parameters. All potential junction reads were counted and normalized by the size factor (the ratio of uniquely-mapped reads of the target tissue and tissue with minimal sequencing).

1.6 Generation of Antibodies Specific for Exon 16 or Exon 21 of CaMKII-δ

To produce antibodies in rabbits, peptides were commercially synthesized and purified (Abcam, USA). Antigenic epitopes comprised the following amino-acid sequences: PPCIPNGKENFSGGTSLW (SEQ ID NO: 28) corresponding to exon 21, the C terminus unique for a subclass of splice variants of CaMKII-δ, and CEPQTTVIHNPDGNK (SEQ ID NO: 29) corresponding to exon 16 of CaMKII-δ. The internal cysteines were used for conjugation with three different carrier proteins. Each peptide was conjugated with keyhole limpet hemocyanin or ovalbumin to immunize two 3-month-old New Zealand white rabbits. The rabbits were immunized using a customized protocol of 5-6 injections. The original antigens were conjugated to a pre-activated matrix (agarose beads, 1.5 ml) to prepare affinity columns for purification. All the antisera were collected and loaded onto the prepared peptide affinity columns, and eluted with elution buffer (pH 2.7). The eluted poly-antibodies were collected and neutralized based on UV280 absorption.

1.7 Absolute Quantification of CaMKII-δ Splice Variants by Mass Spectrometry

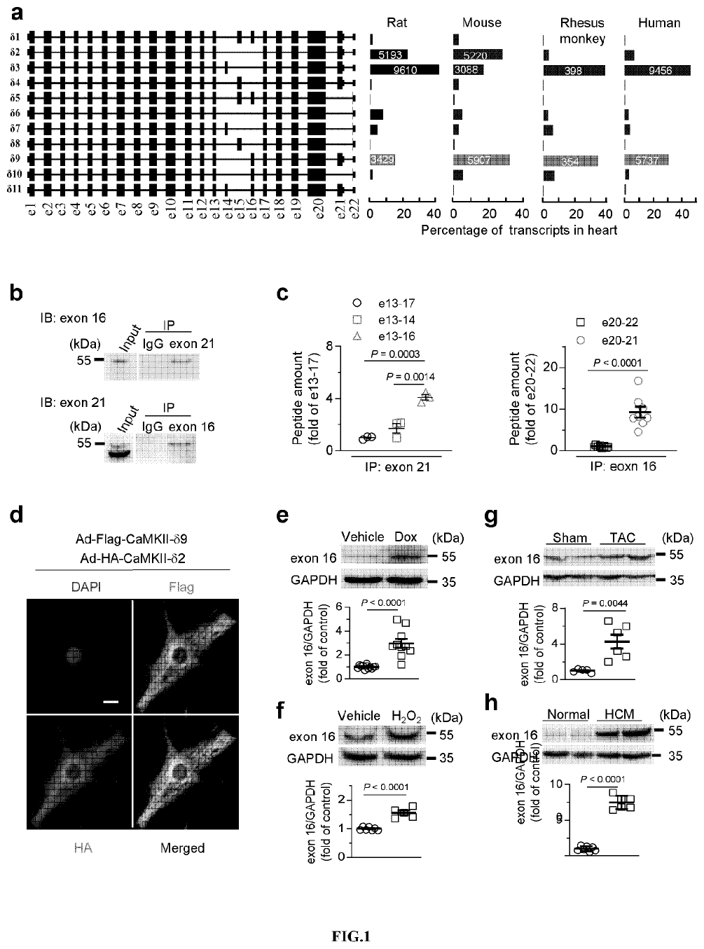

Human left ventricles were homogenized with RIPA buffer, and the lysates were centrifuged at 13,000 rpm for 10 min. CaMKII-δ was immunoprecipitated with the antibody recognizing exon 21 or exon 16, then the agarose pellet was washed and eluted. The eluted protein was subjected to SDS-PAGE, and the band corresponding to CaMKII-δ was cut out for mass spectrometric analysis. Absolute quantification of CaMKII-δ splicing variants were done as previously reported (Gerber, S. A. et al., Proceedings of the National Academy of Sciences of the United States of America 100, 6940-6945, doi:10.1073/pnas.0832254100 (2003); Kawakami, H. et al. Journal of pharmaceutical sciences 100, 341-352, doi:10.1002/jps.22255 (2011)). Specifically, to test the best working condition, in-gel digestion was performed with trypsin (400 ng) for different time (0.5 h, 1 h, 2 h or 4 h), and then subjected to mass spectrometry analysis. 2 h trypsin digestion was chosen since the most unique peptides were observed under this condition, in which several unique peptides were chosen from each target variant as standard peptides for quantification (Table 1). The synthetic peptides contained 13C and 15N labeled lysine. The calibration curve were prepared by serial dilution of synthetic peptides (0.78, 1.56, 3.31, 6.25, 12.5, 25, 50, 100 and 200 fmol) mixed with trypsin digested immunoprecipitated heart sample, followed by mass spectrometry analysis. Based on the loading quantity and the peak area, every synthetic peptide got a linear calibration curve with R2 above 0.992. Quantification values of each target peptide were determined by calculating the ratios of peak areas to those of isotope-labeled peptides (50 fmol spiked standard). In exon 21 immunoprecipitated sample, the average peptide amount is 2.32±0.27 fmol for exons 13-17, 4.02±1.50 fmol for exons 13-14, and 9.52±0.88 fmol for exons 13-16. In exon 16 immunoprecipitated sample, the average peptide amount is 11.87±3.73 fmol for exons 20-21, and 1.73±0.71 fmol for exons 20-22. The relative data is shown in FIG. 1c.

TABLE 1
Identified proteotypic trypsin peptides for quantitative mass
spectrometry in the hearts

| Exon junctions | Amino acid sequence |
|---|---|
| e20-21 | SGSPTVPIKPPOPNGK* (SEQ ID NO: 30) |
| e20-22 | SGSPTVPIIK* (SEQ ID NO: 31) |
| e13-17 | KPDGVKESTESSNTTIEDEDVK* (SEQ ID NO: 32) |
| e13-14 | SLLKKPDGVKK* (SEQ ID NO: 33) |
| e13-16 | KPDGVKEPQTTVIEINPDGNK* (SEQ ID NO: 34) |

Star means the amino acid with heavy isotope labeled.

All samples were analyzed using a Q-Exactive HF (Thermo) mass spectrometer with an Easy-nLC1000 liquid chromatography system (Thermo). Peptides were eluted from a 100-μm ID×2 cm, C18 trap column and separated on a homemade 150 μm ID×15 cm column (C18 resin, 1.9 m, 120 Å, Dr. Maisch GmbH) with a 75-min linear 5-35% acetonitrile gradient at 600 nL/min. The MS analysis for Q-Exactive HF was performed with one full scan (300-1400 m/z, R=120,000 at 200 m/z) at an automatic gain control target of 3e6 ions, followed by up to 20 data-dependent MS/MS scans with higher-energy collision dissociation (AGC target 2e4 ions, max injection time 40 ms, isolation window 1.6 m/z, normalized collision energy of 27%), detected in the Orbitrap (R=15,000 at 200 m/z). The dynamic exclusion of previously-acquired precursor ions was enabled at 12 s.

Peptides were identified using Proteome Discoverer Software (version 1.4.1.14, Thermo) suited with Mascot software (version 2.3.01, Matrix Science) to achieve a false discovery rate of <1%. The mass tolerance was set to 20 ppm for precursors, and to 50 mmu for the tolerance of product ions. Oxidation (M), Acetyl (Protein-N term), and DeStreak (C) were chosen as variable modifications; and two missed cleavages on trypsin were allowed.

1.8 Mass Spectrometric Analysis

For the analysis of UBE2T phosphorylation, HEK293 cells were transfected with myc-tagged UBE2T in the presence of control vector or flag-tagged CaMKII-δ9 plasmid. The proteasome inhibitor MG132 was added 12 h before cell harvesting, then the total proteins were extracted with RIPA buffer. Total UBE2T was immunoprecipitated with anti-myc antibody, then the agarose pellet was washed and eluted. The eluted protein was subjected to SDS-PAGE, and the band corresponding to UBE2T was cut out for mass spectrometric analysis.

In LC-MS/MS analysis, digestion products were separated by a 65-min gradient elution at a flow rate of 0.3 μL/min with the Dionex 3000 nano-FPLC system, which was directly interfaced with a Thermo LTQ Orbitrap Velos Pro mass spectrometer. The analytical column was a fused silica capillary column (75 μm ID, 150 mm long; packed with C18 resin). Mobile phase A consisted of 0.1% formic acid, and mobile phase B consisted of 80% acetonitrile and 0.08% formic acid. The mass spectrometer was operated in the data-dependent acquisition mode using Xcalibur2.1.3 software and there was a single full-scan mass spectrum in the Orbitrap (400-1800 m/z, 30,000 resolution) followed by 10 data-dependent MS/MS scans. The MS/MS spectrum from each LC-MS/MS run was searched against the selected database using the Proteome Discovery search algorithm (version 1.3).

1.9 Comet Assay

Cultured neonatal rat ventricular cardiomyocytes (NRVMs) were treated as indicated, and then washed with cold PBS, following the protocol of the comet assay kit (Trevigen, Cat #: 4250-050-K) under alkaline conditions. Mean tail moments were quantified for 300-500 cells per sample in each experiment, with Comet Assay Software Project (v1.2.3b1).

1.10 Generation of CaMKII-δ9 tg Mice

The full-length human CaMKII-δ9 cDNA coding sequence with an N-terminal Flag tag was cloned into the HindIII and EcoRV sites of an expression vector containing the α-MHC promoter. After linearization with XhoI and NotI, gel-purification was performed. This construct was microinjected into the pronuclei of fertilized C57BL/6J mouse eggs. PCR was used for genotyping. The primer sequences were 5′-GTATCGATAAGCTTGCCACCATGG-3′ (forward) (SEQ ID NO: 35) and 5′-CATGAAGTCGCACAATATTAGG-3′ (reverse) (SEQ ID NO: 36).

1.11 Generation of CaMKII-δ9 shRNA tq Mice

The sequence of miR30-based CaMKII-δ9 shRNA was 5′-GAAGGTATATTGCTGTTGACAGTGAGCGCAATCCACAACCCTGACGGAAATAGTG AAGCCACAGATGTATTTCCGTCAGGGTTGTGGATTATGCCTACTGCCTCGG-3′ (SEQ ID NO: 37). The shRNA with an N-terminal EGFP tag and C-terminal BGH poly A sequences was cloned into the pLKO.1 plasmid containing the U6 promoter. After linearization, gel-purification was performed. This construct was microinjected into the pronuclei of fertilized C57BL/6J mouse eggs. PCR was used for genotyping. The primer sequences were 5′-CTTCACCGAGGGCCTATTTCC-3′ (forward) (SEQ ID NO: 38) and 5′-CCGTAGGTGGCATCGCCCTC-3′ (reverse) (SEQ ID NO: 39).

1.12 Generation of CaMKII-δ2 tg Mice

The full-length human CaMKII-δ2 cDNA coding sequence with an N-terminal HA tag was cloned into the HindIII and EcoRV sites of an expression vector containing the α-MHC promoter. After linearization with XhoI and NotI, gel-purification was performed. This construct was microinjected into the pronuclei of fertilized C57BL/6J mouse eggs. PCR was used for genotyping. The primer sequences were 5′-CGGTATCGATAAGCTTGGCC-3′ (forward) (SEQ ID NO: 40) and 5′-TCACAATATTGGGGTGCTTC-3′ (reverse) (SEQ ID NO: 41).

1.13 Generation of UBE2T tz Mice

The full-length rat UBE2T cDNA coding sequence with a C-terminal myc tag was cloned into the HindIII and EcoRV sites of an expression vector containing the α-MHC promoter. After linearization with XhoI and NotI, gel-purification was performed. This construct was microinjected into the pronuclei of fertilized C57BL/6J mouse eggs. PCR was used for genotyping. The primer sequences were 5′-ATAGAAGCCTAGCCCACACC-3′ (forward) (SEQ ID NO: 42) and 5′-GATCTGTGGTGGCTCAAATG-3′ (reverse) (SEQ ID NO: 43).

1.14 Echocardiography

Echocardiographic analysis using a Vevo2100 digital imaging system (Visual Sonics, Toronto, ON, Canada) was performed under 1% isoflurane at 6 and 10 weeks of age, with mid-ventricular M and B mode measurements acquired in the parasternal short-axis view at the level of the papillary muscles. Once the mice were acclimated to the procedures, images were stored in digital format on a magnetic optical disk for review and analysis. Measurements of the LV internal end-diastolic diameter (LVIDd) were made at the time of apparent maximal LV diastolic dimension, while measurements of the LV internal end-systolic diameter (LVIDs) were taken at the time of the most anterior systolic excursion of the posterior wall. LV ejection fraction (EF) was calculated by the cubic method: LVEF (%)={(LVIDd)³−(LVIDs)³}/(LVIDd)³×100, and LV fractional shortening (FS) was calculated by FS (%)=(LVIDd−LVIDs)/LVIDd×100. The data were averaged from 5 cardiac cycles.

1.15 Histological Analysis

Histological analysis of heart tissue was as previously described (Zhang, T. et al. Nature medicine 22, 175-182, doi: 10.1038/nm.4017 (2016)). The CardioTACS™ in situ apoptosis detection kit (Roche Applied Science, Cat #: 11684795910) was used for TUNEL staining as previously described (Zhang, T. et al. Nature medicine 22, 175-182, doi:10.1038/nm.4017 (2016)).

1.16 Gene Expression Analysis and Primers

The primer pairs used for quantitative real-time PCR were in Table 2. Amplification was performed as follows: 95° C. for 3 s and 40 cycles at 95° C. for 15 s and 60° C. for 30 s. Data are the average of at least three independent experiments.

TABLE 2
The primer pairs used for quantitative real-time PCR analysis of gene expression

| Gene | Direction | Sequence 5′-3′ |
|---|---|---|
| 18S | Forward | GGAAGGGCACCACCAGGAGT (SEQ ID NO: 44) |
| | Reverse | TGCAGCCCCGGACATCTAAG (SEQ ID NO: 45) |
| a-MHC | Forward | ATCATTCCCAACGAGCGAAAG (SEQ ID NO: 46) |
| | Reverse | AAGTCCCCATAGAGAATGCGG (SEQ ID NO: 47) |
| ANP | Forward | TTCTTCCTCGTCTTGGCCTTT (SEQ ID NO: 48) |
| | Reverse | GACCTCATCTTCTACCGGCATCT (SEQ ID NO: 49) |
| ATF3 | Forward | CATCAACAACAGACCTCT (SEQ ID NO: 50) |
| | Reverse | CATTCACACTCTCCAGTT (SEQ ID NO: 51) |
| Bdkrb1 | Forward | CCTTCTACGCTCTGTTAA (SEQ ID NO: 52) |
| | Reverse | CCAGGATGTGATAGTTGAA (SEQ ID NO: 53) |
| b-MHC | Forward | ATGTGCCGGACCTTGGAA (SEQ ID NO: 54) |
| | Reverse | CCTCGGGTTAGCTGAGAGATCA (SEQ ID NO: 55) |
| BNP | Forward | AAGTCCTAGCCAGTCTCCAGA (SEQ ID NO: 56) |
| | Reverse | GAGCTGTCTCTGGGCCATTTC (SEQ ID NO: 57) |
| CaMKII-d2 | Forward | CCAGATGGGGTAAAGGAGTCAACTGAGAGCT (SEQ ID |
| | | NO: 58) |
| CaMKII-d2/3/9 | Reverse | TCAGATGTTTTGCCACAAAGAGGTGCCTCCT (SEQ ID |
| | | NO: 59) |
| CaMKII-d3 | Forward | AAAAGGAAGTCCAGTTCGAGTGTTCAGATGAT (SEQ |
| | | ID NO: 60) |
| CaMKII-d9 | Forward | GTAAAGGAGCCCCAAACTACTGTAA (SEQ ID NO: 61) |
| Cdo1 | Forward | GACCTCATCCGAATCTTG (SEQ ID NO: 62) |
| | Reverse | CAGCACAGAATCATCAGA (SEQ ID NO: 63) |
| Chac1 | Forward | GCTACGACACTAAGGAAG (SEQ ID NO: 64) |
| | Reverse | CGCAACAAGTATTCAAGG (SEQ ID NO: 65) |
| COX-2 | Forward | GGCACAAATATGATGTTCGCA (SEQ ID NO: 66) |
| | Reverse | CCTCGCTTCTGATCTGTCTTGA (SEQ ID NO: 67) |
| CSF2 | Forward | CTATACAAGCAGGGTCTAC (SEQ ID NO: 68) |
| | Reverse | CTATTTCACAGTCAGTTTCC (SEQ ID NO: 69) |
| Ccna2 | Forward | GAAGGTCTCAGGTTATCAG (SEQ ID NO: 70) |
| | Reverse | GTCTGGTCATTCTGTCTC (SEQ ID NO: 71) |
| Ereg | Forward | AGAGAAGGATGGAGACTT (SEQ ID NO: 72) |
| | Reverse | GCTGATAACTGCTTGTAGA (SEQ ID NO: 73) |
| Has1 | Forward | AACTGGCTGCTAACTATG (SEQ ID NO: 74) |
| | Reverse | AGTCTCCTTACACCTACC (SEQ ID NO: 75) |
| Hpgd | Forward | CAGGAGTGAACAATGAGA (SEQ ID NO: 76) |
| | Reverse | ATAAGGTTAGCAGCCATC (SEQ ID NO: 77) |
| LIF | Forward | GATTTCCCACCTTTCCAT (SEQ ID NO: 78) |
| | Reverse | CTGTAGTCGCATTGAGTT (SEQ ID NO: 79) |
| PAI-2 | Forward | AATCCATTCAGCCTTCTC (SEQ ID NO: 80) |
| | Reverse | CAGCGTTGTATGTATTCTTC (SEQ ID NO: 81) |
| Plk1 | Forward | TATTACCTGCCTCACCAT (SEQ ID NO: 82) |
| | Reverse | CTTGTCCGAATAGTCTACC (SEQ ID NO: 83) |
| Reg3b | Forward | AATATACCTGGATTGGACTC (SEQ ID NO: 84) |
| | Reverse | CAATGTGGACTGTAGATAGA (SEQ ID NO: 85) |
| UBE2T | Forward | AGCCAATACACCTTATGAG (SEQ ID NO: 86) |
| | Reverse | TCCTTCCACTAGAATCAATG (SEQ ID NO: 87) |

1.17 Plasmid Construction

A vector expressing CaMKII-δ9 was cloned from the cDNA of the human left ventricle. Rat UBE2T was cloned from the cDNA of rat heart; plasmid with the S110A point mutation or S913A mutation of rat UBE2T was generated using the Stratagene QuikChange II site-directed mutagenesis kit. Plasmids expressing exons 13-15-16-17, 13-17, 13-14-17, and 13-16-17, the feature sequences of CaMKII-δ1, 62, 63, and 69, respectively, were cloned with a GFP tag to pcDNA5/flag plasmid. Plasmid was transfected when HEK 293 cells reached 80% confluence.

Adenoviral vector expressing human CaMKII-δ9, rat UBE2T, rat UBE2T-S110A, or rat UBE2T-S193A was constructed by Sino Geno Max Co., Ltd. The adenoviral vector expressing CaMKII-δ2 was as previously described (Zhu, W. et al., The Journal of biological chemistry 282, 10833-10839, doi:10.1074/jbc.M611507200 (2007)).

Plasmid expressing human UBE2T was from OriGene (expression plasmid of Cat #: TP300748), and the S110A point mutation was generated using the Stratagene QuikChange II site-directed mutagenesis kit. A positive clone with the S110A point mutation was confirmed by sequencing and then the plasmid was amplified for protein purification.

1.18 Isolation, Culture, and Adenoviral Infection of Ventricular Myocytes

NRVMs were isolated from 1-day-old Sprague-Dawley rats, and adenovirus-mediated gene transfer was implemented using methods described previously (Zhang, T. et al., Nature medicine 22, 175-182, doi: 10.1038/nm.4017 (2016)). NRVMs were exposed to H₂O₂ (200 μM) or Dox (1 μM) for 24 h.

1.19 Isolated Mouse Heart Perfusion

Adult mice (10-12 weeks old) were anesthetized by intraperitoneal injection of pentobarbital (70 mg/kg). The heart was excised and perfused on a Langendorff apparatus at a constant pressure of 55 mmHg. The buffer was continuously gassed with 95% O₂/5% CO₂ (pH 7.4) and warmed by a heating bath/circulator. The heart temperature was continuously monitored and maintained at 37±0.5° C. Global ischemia was induced by cessation of perfusion for 30 min followed by reperfusion.

1.20 Subcellular Fractionation

Cytosolic and nuclear proteins were separated with a Nuclear/Cytosolic Fractionation Kit (Biovision Research Products, Cat #: K266, USA) following the manufacturer's instructions.

1.21 Cell Viability Analysis

Cardiomyocyte viability was assayed by caspase 3/7 activity and LDH concentration in the culture medium as previously described (Zhang, T. et at., Nature medicine 22, 175-182, doi:10.1038/nm.4017 (2016)). Caspase 3/7 activity was measured with a kit from Promega (Cat #: G8091) according to the manufacturer's instructions. The LDH concentration in the culture medium was spectrophotometrically assayed using a kit from Sigma (Cat #: MAK066).

1.22 Heart and Cardiomvocyte Histology

Hearts were fixed overnight in 4% paraformaldehyde (pH 7.4), embedded in paraffin, and serially sectioned at 5 μm. Standard hematoxylin and eosin staining or immunohistochemistry was performed on these sections.

Cardiomyocyte immunofluorescence was measured as previously described (Erickson, J. R. et al., Physiological reviews 91, 889-915, doi:10.1152/physrev.00018.2010 (2011)).

1.23 Western Blot and Co-Immunoprecipitation

Western blot and co-immunoprecipitation were performed as previously described (Zhang, T. et al., Nature medicine 22, 175-182, doi:10.1038/nm.4017 (2016)).

1.24 RNA Interference-Mediated Gene Silencing

For gene-silencing assays, siRNAs 19 nucleotides in length, with a dTdT overhang at the 3′ terminus, were designed using the Invitrogen website. Cardiomyocytes were transfected with siRNA using Lipofectamine RNAiMAX (Invitrogen) following the manufacturer's instructions (Erickson, J. R. et al., Physiological reviews 91, 889-915, doi:10.1152/physrev.00018.2010 (2011)). The efficiency of gene-knockdown was assessed by western blot 72 h after siRNA transfection. The sequences of the siRNAs were in Table 3.

TABLE 3
The target sequences of siRNAs.

| Gene | Sequence 5′-3′ |
|---|---|
| Scrambled | UCCCAAUCCUAGGGACAAA (SEQ ID NO: 88) |
| CaMKII-d9 | UCCACAACCCUGAUGGAAA (SEQ ID NO: 89) |
| COX-2 siRNA1 | CCUUCCUUCGGAAUUCAAU (SEQ ID NO: 90) |
| COX-2 siRNA2 | CCAUGGGUGUGAAAGGAAA (SEQ ID NO: 91) |
| COX-2 siRNA3 | CCAGUAUCAGAACCGCAUU (SEQ ID NO: 92) |
| UBE2T siRNA1 | CCACUGUAUUGACCUCUAU (SEQ ID NO: 93) |
| UBE2T siRNA2 | CCAUGCAGCGAUUCUUUAA (SEQ ID NO: 94) |
| FANCD2 siRNA1 | GCGGCUGAACAUAAGGCUU (SEQ ID NO: 95) |
| FANCD2 siRNA2 | GCUGUCAUCUGUCCGUCUA (SEQ ID NO: 96) |
| FANCI siRNA1 | CCUGAGAGCCAUCCUCAAA (SEQ ID NO: 97) |
| FANCI siRNA2 | CCAUCAUCCUUACUGCCUU (SEQ lD NO: 98) |

1.25 Cell-Free Kinase Assay of CaMKII-δ

Human CaMKII-δ2 protein was from Abcam (Cat #: ab84552), human UBE2T protein from OriGene (Cat #: TP300748), and human CaMKII-δ9 and UBE2T-S110A proteins were produced by OriGene. Cell-free kinase assays were performed in a kinase buffer containing 100 mM Tris-HCl, pH 7.5, 20 mM MgCl₂, and 4 mM DTT. CaMKII-δ9 or 62 protein was incubated with 200 μM CaCl₂) and 1 M CaM (Sigma) on ice for 1 min, then exposed to 1 mM ATP for 30 min at 30° C. in the presence of UBE2T protein. Reactions were stopped by adding SDS loading buffer. Samples were boiled for 5 min and separated on SDS-PAGE. Commercial antibodies against phosphorylated serine and UBE2T were used for immunoblot analysis.

1.26 Human Embryonic Stem Cells Induced into Cardiomyocytes

Human embryonic stem cells H9 were differentiated into cardiomyocytes using a chemically-defined, xeno-free, small molecule-based method as previously reported (Burridge, P. W. et al., Nature methods 11, 855-860, doi:10.1038/nmeth.2999 (2014)). Briefly, H9 cells were maintained on a 6-well plate pre-coated with Matrigel (BD Biosciences, Cat #: 354277) in E8 medium (Life Technologies, Cat #: A1517001). When the cells grew to 70% confluence, the medium was replaced with basal medium supplemented with 6 μM CHIR99021 (Selleckchem, Cat #: S1263-25 mg); 48 h later, the medium was changed to basal medium supplemented with 2 μM Wnt-C59 (Biorbyt, Cat #: orb181132) for another 48 h. Then the cells were maintained in basal medium, changing to fresh medium every 48 h. Beating cardiomyocytes emerged at about day 8. On day 10, the basal medium was replaced with RPMI 1640 without glucose (Life Technologies, Cat #: 11879020) to purify the cardiomyocytes. Two days later, the cardiomyocytes were associated with TrypLE™ Express Enzyme (Life Technologies) for 10 min and passaged to a Matrigel pre-coated well plate.

1.27 Materials

Antibodies against the following proteins were used: rat/human UBE2T and p-threonine (Cell Signaling Technology, 12992 (Lot #: 1; 1:1000) and 9381 (Lot #: 22; 1:1000)); mouse UBE2T (Aviva Systems Biology, ARP-43145 (Lot #: QC13585-40506; 1:1000)); p-CaMKII (Thermo, MA1-047 (Lot #: QC207772; 1:1000)), t-CaMKII-δ (GeneTex, GTX111401 (Lot #: 40058; 1:1000)), Myc and Flag (Sigma, SAB4700447 (Lot #: 522137; 1:5000 for western blots, 1:200 for immunohistochemistry), and F1804 (Lot #: SLBR7936V; 1:5000 for western blots, 1:200 for immunohistochemistry)), γH2AX, p-serine, and ox-CaMKII (Millipore, 05-636, clone JBW301 (Lot #: 2884537; 1:1000 for western blots, 1:200 for immunohistochemistry), 05-1000, clone 4A4 (Lot #: 2691195; 1:1000) and 07-1387 (Lot #: 2739150; 1:1,000)), COX-2, HA, and lamin A/C (Santa Cruz, sc-1747 (Lot #: H1911; 1:1000), sc-7392 (Lot #: L1115; 1:1000 for western blots, 1:200 for immunohistochemistry) and sc-6215 (Lot #: J2615; 1:1000)), PAI-2 (Bioworld, BS3702 (Lot #: CA36131; 1:1000)), f-actin (EARTHOX, E021070-01 (Lot #:0a1401, 1:100 for immunohistochemistry)), FANCD2 and FANCI (abcam, ab108928 (Lot #: GR130039-32; 1:1000) and ab74332 (Lot #: GR251812-8; 1:1000)), and GAPDH (EASYBIO, BE0023, clone 2B8 (1:10000)). MG132, clasto-lactacystin β-lactone (β-lac), Doxorubicin, and H₂O₂ were from Sigma-Aldrich.

1.28 Statistics and Reproducibility

Data are expressed as mean±s.e.m. Statistical analysis was performed with GraphPad Prism version 5.01 (GraphPad Software, Inc.) and the SPSS 18.0 software package (SPSS Inc.). Data sets were tested for normality of distribution with the Kolmogorov-Smirnov test. Data groups (two groups) with normal distributions were compared using the two-sided unpaired Student's t-test. The Mann-Whitney U-test was used for nonparametric data. Comparisons between multiple groups were assessed by one-way or two-way ANOVA with Tukey's multiple comparisons test. No statistical method was used to predetermine sample size.

### Example 1. The Presence of CaMKII-δ9 in Human Heart

The inventors performed single-molecular real-time (SMRT) sequencing (Pacific Biosciences) (Sharon, D. et al., Nature biotechnology 31, 1009-1014, doi:10.1038/nbt.2705 (2013)) of cardiac tissue from mouse, rat, rhesus monkey, and human to detect the concentrations of splice variants of CaMKII-δ. The library preparation, sequencing, and data collection for SMRT sequencing were described in Section 1.3 of GENERAL METHODS AND MATERIALS. The quantification of CaMKII-δ splice variants was performed according to the methods as described in Section 1.7 of GENERAL METHODS AND MATERIALS.

Surprisingly, the inventors found that the well-studied splice variant CaMKII-δ2 was extremely low in the hearts of rhesus monkey (3.1%) and human (6.3%), although it accounted for 29% and 22.5% of the total cardiac CaMKII-δ transcripts in mouse and rat, respectively. The previously-reported but functionally-overlooked CaMKII-δ9 emerged as a very abundant splice variant, accounting for 33.5%, 31.9%, 32.9% and 14.9% in rhesus monkey, human, mouse and rat, respectively, comparable in all cases to CaMKII-δ3 and much higher than 62 in primates (FIG. 1a).

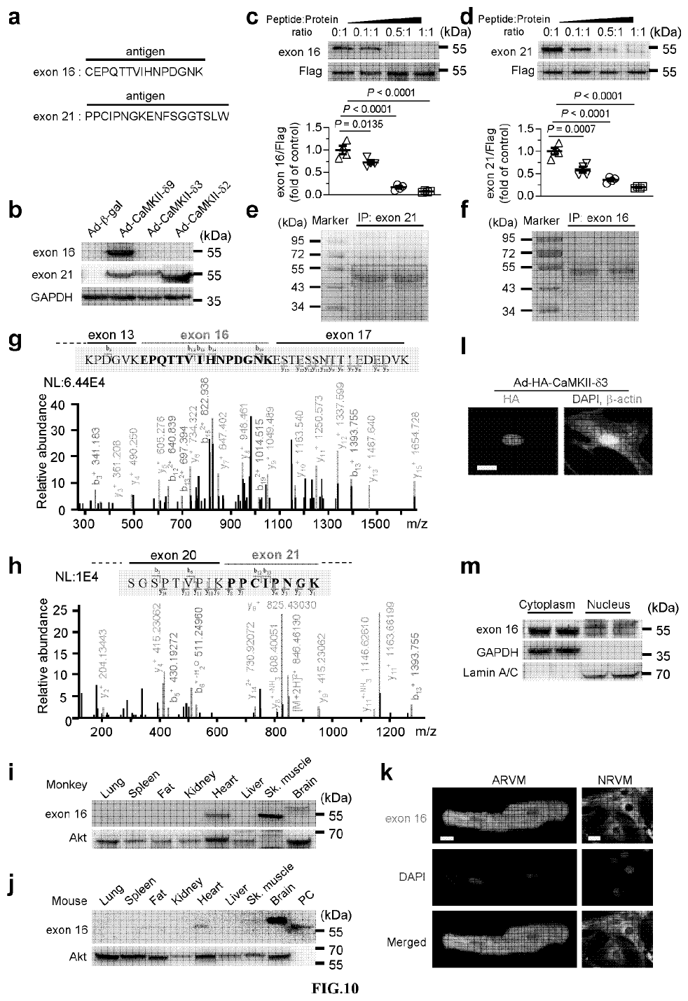

Additionally, cardiac CaMKII-δ9 expression at the protein level was detected by two customized antibodies that reacted with the peptides corresponding to exon 16 (anti-exon 16) and exon 21 (anti-exon 21), respectively. The antibodies were prepared according to the methods as described in Section 1.6 of GENERAL METHODS AND MATERIALS. A single band close to 50 kD was revealed by immunoprecipitation of mouse myocardial proteins with either antibody followed by immuno-blot assay with the other antibody. Mass spectrometry (MS) analysis of the band around 50 kD immunoprecipitated by anti-exon 21 from mouse hearts identified a peptide encoded by exons 13-16-17, the feature sequence of CaMKII-δ9 (FIG. 10e, g). Alternatively, a peptide encoded by exons 20-21 was detected in samples immunoprecipitated by anti-exon 16 (FIG. 10f, h). Importantly, the inventors performed absolute quantitative MS analysis (Gerber, S. A. et al., Proceedings of the National Academy of Sciences of the United States of America 100, 6940-6945, doi:10.1073/pnas.0832254100 (2003); Kawakami, H. et al. Journal of pharmaceutical sciences 100, 341-352, doi:10.1002/jps.22255 (2011)) of human cardiac tissues (Table 1), and found that, when immunoprecipitated with anti-exon 21, the levels of the peptides containing exons 13-16 (69), and 13-14 (63, and 611) were 4.1- and 1.7-fold that of exons 13-17 (62) (FIG. 1c). Similarly, when immunoprecipitated with anti-exon 16, the level of the peptide encoded by exons 20-21 (61, 69 and 611) was 9.2-fold of that encoded by exons 20-22 (65 and 610) (FIG. 1c), distinguishing CaMKII-δ9 from 610. Thus, the inventors conclude that CaMKII-δ9 constitutes a major cardiac CaMKII-δ splice variant in mammals at both the mRNA and protein levels, particularly in nonhuman primates and humans.

To determine the tissue distribution of CaMKII-δ9, using anti-exon 16, the inventors found that CaMKII-δ9 was detected in striated muscle, both heart and skeletal muscle, in rhesus monkeys (FIG. 10i). However, in mice, CaMKII-δ9 was specifically expressed in the heart (FIG. 10j). Immunofluorescence imaging revealed a cytosolic distribution pattern of CaMKII-δ9 in cardiomyocytes (FIG. 1d and FIG. 10k), partially overlapping with CaMKII-δ2 (FIG. 1d), while CaMKII-δ3 was enriched in the nuclear compartment (FIG. 10l). The cytosolic distribution of CaMKII-δ9 was confirmed by western blots of subcellular fractions (which was performed according to the methods as described in Section 1.20 of GENERAL METHODS AND MATERIALS) of cultured neonatal rat ventricular cardiomyocytes (NRVMs) (FIG. 10m). Therefore, the inventors conclude that CaMKII-δ9 constitutes an important CaMKII-δ splice variant with cytosolic distribution in the heart of mammals, especially nonhuman primates and humans.

### Example 2. Upregulation of CaMKII-δ9 is Associated with Various Cardiac Diseases

To investigate the pathological relevance of CaMKII-δ9, the inventors first evaluated its levels in several cardiac injury models. To mimic hemodynamic pressure overload, the inventors performed transverse thoracic constriction (TAC) surgery in mice. The experimental methods, animals, and materials used in this Example were described in Sections 1.1, 1.2, 1.4, 1.15, 1.18, 1.19, 1.26 and 1.27 of GENERAL METHODS AND MATERIALS.

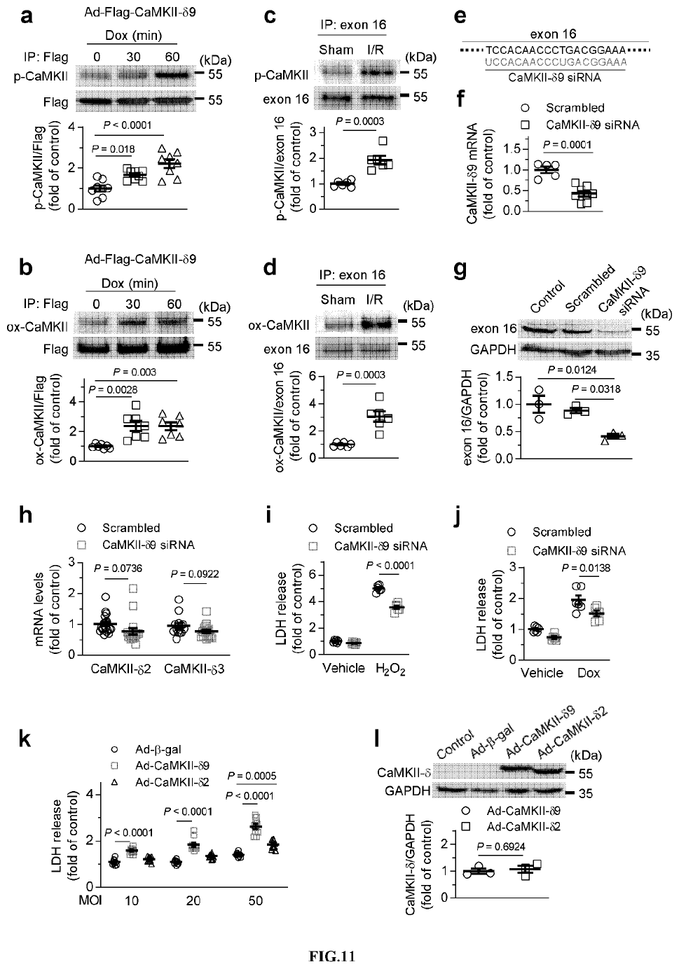

The expression of CaMKII-δ9 was significantly elevated in NRVMs exposed to the chemotherapeutic drug Doxorubicin (Dox; 1 μM), or oxidative stress with H₂O₂ (200 μM) (FIG. 1e, f). The protein level of CaMKII-δ9 was overtly increased in TAC hearts relative to the sham group (FIG. 1g). More importantly, CaMKII-δ9 was profoundly elevated in cardiac tissue from patients with hypertrophic cardiomyopathy (HCM) relative to controls (FIG. 1h). It is known that CaMKII is activated through both phosphorylation and oxidation (Erickson, J. R. et al., Physiological reviews 91, 889-915, doi: 10.1152/physrev.00018.2010 (2011); Erickson, J. R. et al., Cell 133, 462-474, doi:10.1016/j.cell.2008.02.048 (2008)). Acute Dox treatment increased both the phosphorylation and oxidation levels of CaMKII-δ9 in cardiomyocytes (FIG. 11a, b). Moreover, CaMKII-δ9 phosphorylation and oxidation were also increased in mouse hearts subjected to acute ischemia-reperfusion injury (30 min ischemia followed by 60 min reperfusion) (FIG. 11c, d). Thus, cardiac CaMKII-δ9 is upregulated and hyper-activated in response to a variety of pathological stresses, underscoring the pathological relevance of CaMKII-δ9 in the heart.

### Example 3. Enhanced CaMKII-δ9 Signaling Triggers Cardiomyocyte Death

Next, the inventors sought to determine the possible role of CaMKII-δ9 in the regulation of cardiac cell fate. The inventors designed an siRNA targeting exon 16 of CaMKII-δ to specifically reduce the CaMKII-δ9 level (FIG. 11e-g) without altering the expression levels of CaMKII-δ2 or 63 in cardiomyocytes (FIG. 11h). The siRNA was designed according to the method as described in Section 1.24 of GENERAL METHODS AND MATERIALS. The cell viability analysis was conducted according to the methods as described in Section 1.21 of GENERAL METHODS AND MATERIALS.

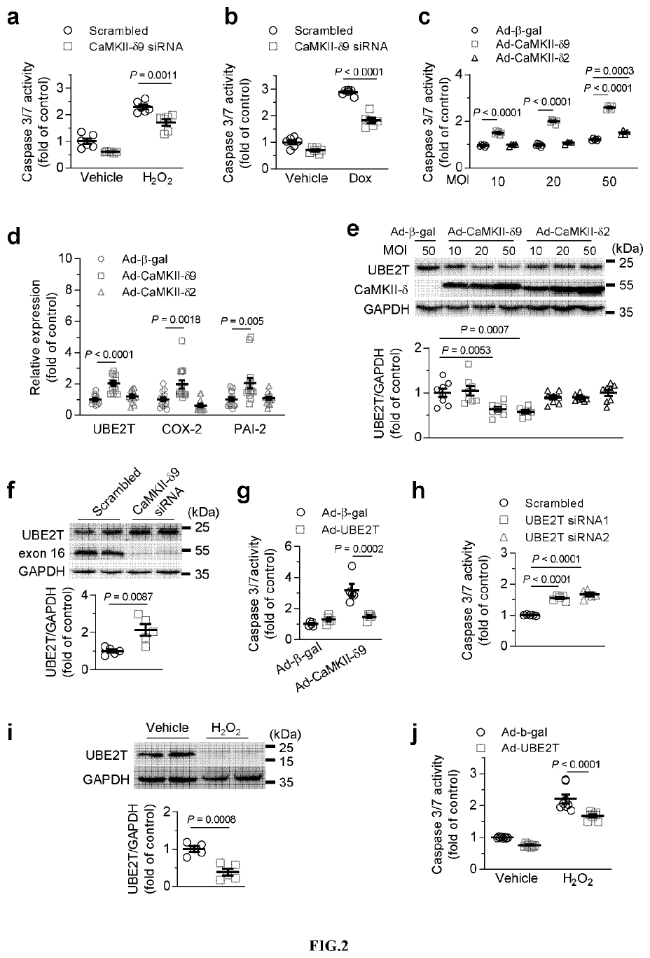

It was found that knockdown of CaMKII-δ9 significantly alleviated both the H₂O₂- and Dox-induced cardiomyocyte death, as indexed by caspase 3/7 activity and lactate dehydrogenase (LDH) concentration in the culture medium (FIG. 2a, b and FIG. 11i, j), suggesting that CaMKII-δ9 is involved in cardiac injury induced by oxidative stress and Dox. Furthermore, adenoviral gene transfer of CaMKII-δ9 was sufficient to cause robust cardiomyocyte death in a titer-dependent manner (FIG. 11k). It is also noteworthy that, when overexpressed at a matched level (FIG. 11l), CaMKII-δ9 was much more potent in inducing cardiomyocyte death than CaMKII-δ2 (FIG. 11k), marking CaMKII-δ9 as an important pathogenic factor involved in oxidative injury and hypertrophic cardiomyopathy.

### Example 4. CaMKII-δ9 Triggers Cardiomyocyte DNA Damage and Cell Death by Downregulating UBE2T

To decipher the mechanism responsible for CaMKII-δ9-elicited cardiomyocyte death and to distinguish it from that of CaMKII-δ2, the inventors performed RNA-seq analysis on cultured NRVMs overexpressing CaMKII-δ9 or CaMKII-δ2 at a matched protein level. The RNA-seq analysis was performed according to the methods as described in Section 1.5 of GENERAL METHODS AND MATERIALS.

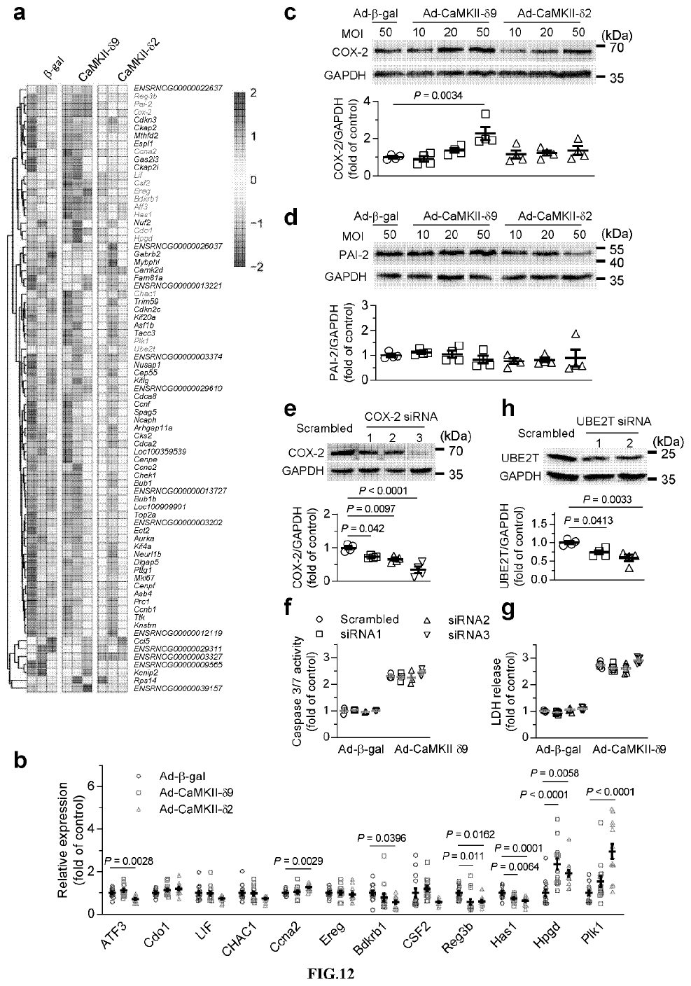

After normalization to the control group (Ad-β-gal), 15 genes were altered by the overexpression of CaMKII-δ9 but not by 62 (FIG. 12a and Table 4). Using real-time PCR (which was performed according to the methods as described in Section 1.16 of GENERAL METHODS AND MATERIALS), the inventors validated the differential regulation of gene expression by CaMKII-δ9 versus CaMKII-δ2. In particular, UBE2T (ubiquitin-conjugating enzyme E2T), COX-2 (prostaglandin G/H synthase 2), and PAI-2 (plasminogen activator inhibitor 2 type A) were upregulated by CaMKII-δ9, but not by 62 (FIG. 2d and FIG. 12b). At the protein level, COX-2 was increased by CaMKII-δ9, but not by 62, consistent with its mRNA level (FIG. 12c), whereas the PAI-2 protein level was unaltered by either CaMKII-δ9 or 62 overexpression (FIG. 12d).

TABLE 4
Gene expression profiles of cardiomyocytes infected with Ad-
CaMII-d9 or Ad-CaMKII-d2 over the control group Ad-b-gal

| | | CaMKII-d9/b-gal | CaMKII-d2/b-gal | |
|---|---|---|---|---|
| Gene Title | Symbol | (fold change) | (fold change) | Function |
| Cyclin A2 | Ccna2 | 1.55 | 1.15 | Function as regulators of |
| | | | | CDK kinases |
| Leukemia | Lif | 1.6 | 0.89 | Induce terminal |
| Inhibitory Factor | | | | differentiation |
| Cysteine | Cdo1 | 1.63 | 1.11 | Initiates metabolic |
| Dioxygenase Type | | | | pathways related to pyruvate |
| 1 | | | | and several sulfurate |
| | | | | compounds |
| 15-hydroxyprostaglandin | Hpgd | 1.76 | 1.26 | Fatty acid metabolism, Lipid |
| dehydrogenase | | | | metabolism, Prostaglandin |
| [NAD(+)] | | | | metabolism |
| Epiregulin | Ereg | 1.82 | 1.01 | Function as a ligand of |
| | | | | EGFR and most members |
| | | | | of the ERBB |
| Plasminogen | PAI-2 | 1.85 | 1.2 | Apoptosis, wound healing |
| activator inhibitor | | | | and endopeptidase |
| 2 type A | | | | |
| Prostaglandin G/H | COX-2 | 1.95 | 1.13 | Cell adhesion, apoptosis and |
| synthase 2 (COX2) | | | | tumor angiogenesis |
| B1 bradykinin | Bdkrb1 | 2.05 | 0.99 | A factor in chronic pain and |
| receptor | | | | inflammation |
| Hyaluronan | Has1 | 2.22 | 1.08 | Hyaluronan biosynthesis and |
| synthase 1 | | | | export |
| Cyclic | Atf3 | 2.33 | 0.87 | Transcription regulation |
| AMP-dependent | | | | |
| transcription factor | | | | |
| ATF-3 | | | | |
| Granulocyte- | Csf2 | 2.67 | 0.96 | Controls the production, |
| macrophage | | | | differentiation, and function |
| colony-stimulating | | | | of granulocytes and |
| factor | | | | macrophages |
| Regenerating | Reg3b | 1.97 | 1.35 | Inflammatory response |
| islet-derived | | | | |
| protein 3-beta | | | | |
| Glutathione-specific | Chac1 | 1.74 | 1.23 | Mediating the pro-apoptotic |
| gamma- | | | | effects of the |
| glutamylcyclotransferase | | | | ATF4-ATF3-DDIT3/CHOP |
| 1 | | | | cascade |
| Ubiquitin-Conjugating | UBE2T | 1.72 | 1.36 | Accepts ubiquitin from the |
| Enzyme E2 T | | | | E1 complex and catalyzes its |
| | | | | covalent attachment to other |
| | | | | proteins |
| PLK1 polo like | Plk1 | 1.52 | 1.24 | Important functions |
| kinase 1 | | | | throughout M phase of the |
| | | | | cell cycle |

Overexpression of CaMKII-δ9 significantly decreased the UBE2T protein level in cultured cardiomyocytes (FIG. 2e), despite an increase in its mRNA level (FIG. 2d); and knockdown of CaMKII-δ9 was able to elevate the UBE2T protein level (FIG. 2f). In contrast, overexpression of the other cytosolic splice variant, CaMKII-δ2, did not alter the protein level of UBE2T in the same experimental setting (FIG. 2e). Furthermore, overexpression of UBE2T rescued the cardiomyocytes from CaMKII-δ9-elicited cell death, as evidenced by caspase 3/7 activity (FIG. 2g), while knockdown of UBE2T was sufficient to trigger cardiac cell death (FIG. 2h and FIG. 12h). In addition, the UBE2T protein level was decreased in NRVMs subjected to oxidative stress (FIG. 2i), and overexpression of UBE2T reduced the H₂O₂-induced cardiomyocyte death (FIG. 2j). Thus, the inventors provide multiple lines of evidence that downregulation of UBE2T plays a crucial role in mediating CaMKII-δ9-induced cardiac cell death.

UBE2T is an ubiquitin-conjugating enzyme (E2) in the Fanconi anemia (FA) DNA repair pathway, which is required for the mono-ubiquitination of FANCD2 and FANCI by FANCL and subsequent DNA repair. The inventors investigated whether the regulation of cardiac cell fate by CaMKII-δ9 is attributable to impairment of the UBE2T-dependent DNA repair pathway and resulting increase in DNA damage. The DNA damage was evaluated by comet assay, which was performed according to the methods as described under Section 1.9 of GENERAL METHODS AND MATERIALS.

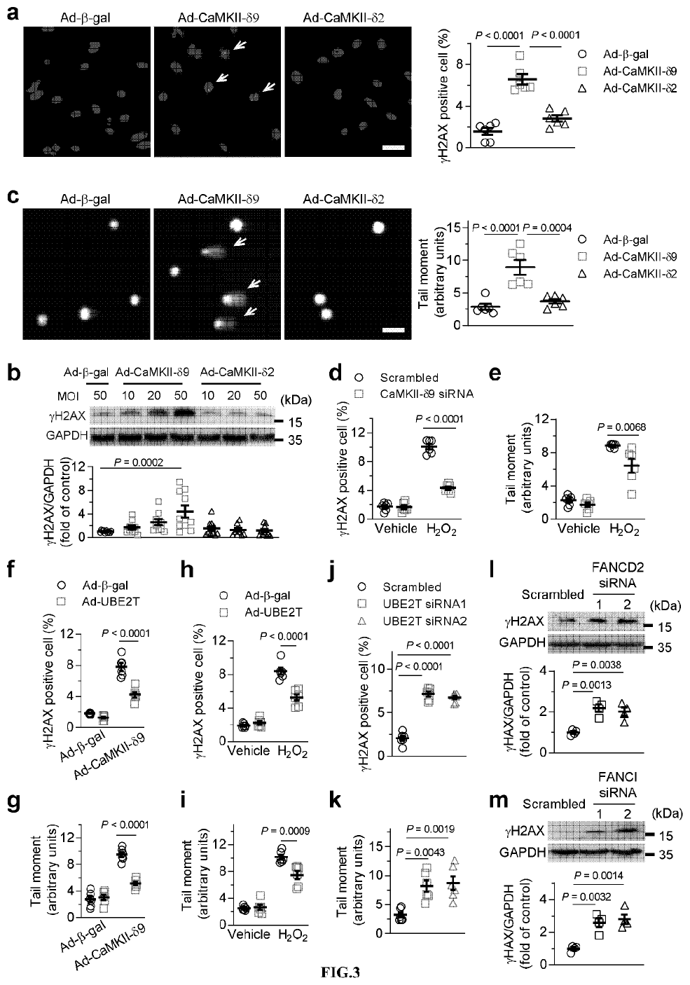

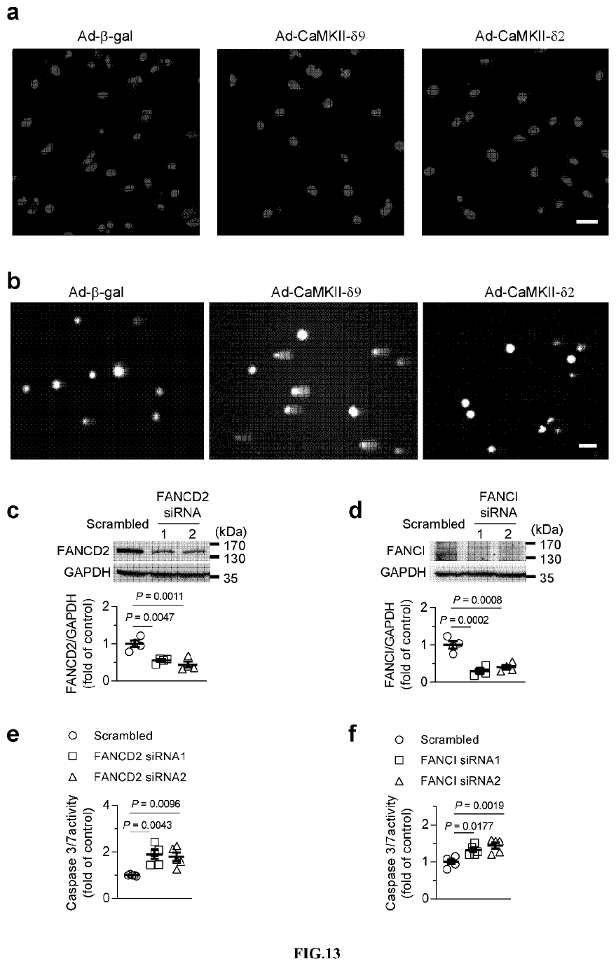

It was found that overexpression of CaMKII-δ9, but not 62, in cardiomyocytes elicited DNA damage, as evidenced by the increased DNA double-strand break marker, γH2AX (Kuo, L. J. & Yang, L. X., In Vivo 22, 305-309 (2008)) and the comet assay (Olive, P. L. & Banath, J. P. Nature protocols 1, 23-29, doi:10.1038/nprot.2006.5 (2006)) (FIG. 3a-c and FIG. 13a, b). In contrast, knockdown of CaMKII-δ9 alleviated oxidative stress-induced DNA damage in cultured cardiomyocytes (FIG. 3d, e), similarly, CaMKII-δ9 deficiency resulted in reduction of cardiac cell death (FIG. 2a, b). Furthermore, UBE2T overexpression reduced the cardiac genome instability induced by CaMKII-δ9 and oxidative stress (FIG. 3f-i), whereas knockdown of UBE2T led to profound DNA damage in cardiomyocytes (FIG. 3j, k). In addition, the inventors found that silencing FANCD2 and FANCI, two downstream molecules of UBE2T, with siRNAs markedly increases the DNA damage, accompanied by augmented cardiomyocyte death (FIG. 3l, m and FIG. 13c-f). Thus, the inventors demonstrated that cardiac cell fate regulation by CaMKII-δ9 is largely attributable to the downregulation of UBE2T and a subsequent increase in DNA damage.

### Example 5. Enhanced CaMKII-δ9-UBE2T-DNA Damage Signaling in Cardiomyopathy and Heart Failure

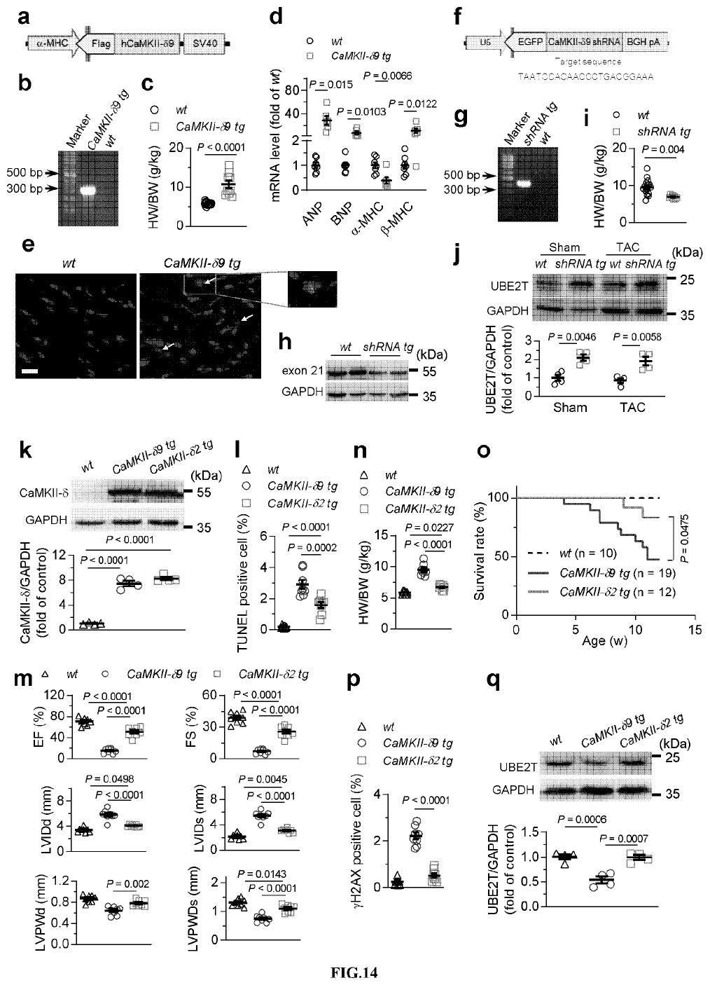

To further evaluate the role of CaMKII-δ9 in cardiac injury and heart failure, the inventors generated transgenic mice with cardiac-specific overexpression of CaMKII-δ9 (CaMKII-δ9 tg) (FIG. 14a, b). The CaMKII-δ9 tg mice and UBE2T tg mice were generated according to the methods as described in Sections 1.10 and 1.13 of GENERAL METHODS AND MATERIALS, respectively.

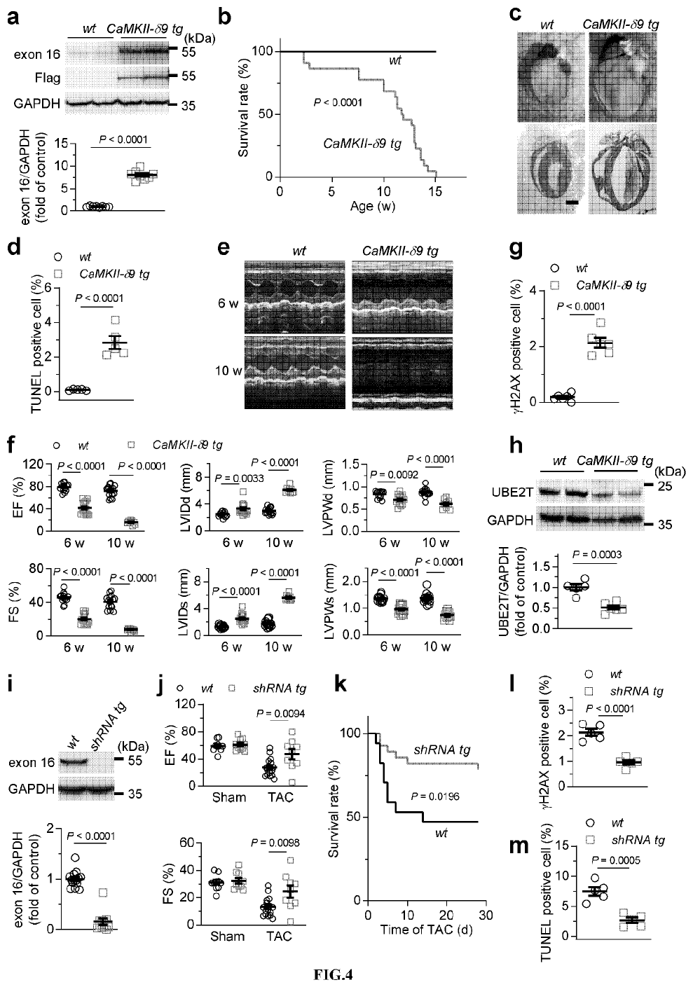

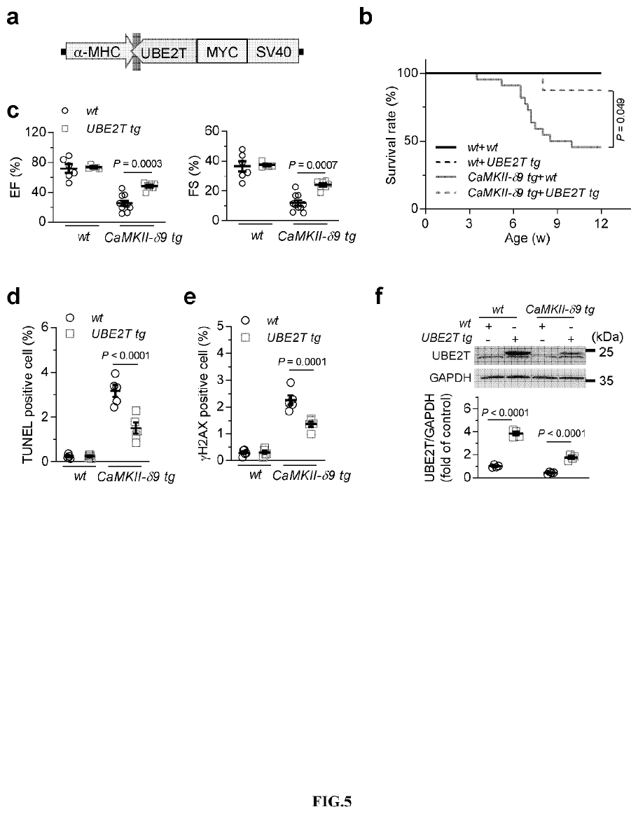

It was found that the CaMKII-δ9 protein level increased by ˜8-fold in the tg mice compared to their wild-type (wt) littermates (FIG. 4a), which was similar to the increase of this splice variant in patients with HCM. The tg mice started to die at 2 weeks of age, and all were dead by 15 weeks, while none of the wt mice died during the same period (FIG. 4b). At 10 weeks of age, the tg mice exhibited profound cardiomyopathy as manifested by cardiac hypertrophy, ventricular dilation, and cardiomyocyte death (FIG. 4c, d). Cardiomyopathy was also displayed by an elevated heart weight to body weight ratio and a hypertrophic gene-expression profile (FIG. 14c, d). As a result, the cardiac function in tg mice deteriorated with age (FIG. 4e, f). By 10 weeks of age, the tg mice developed severe heart failure, as demonstrated by the profoundly depressed ejection fraction (EF) and fractional shortening (FS) compared with wt mice (FIG. 4e, f). Ventricular dilation and cardiac wall thinning in the tg mice were also confirmed by echocardiography (FIG. 4e, f, which was performed according to the methods as described in Section 1.14 of GENERAL METHODS AND MATERIALS). Importantly, in the hearts of CaMKII-δ9 tg mice, DNA damage was overtly increased (FIG. 4g and FIG. 14e), and this was accompanied by a reduction of UBE2T protein abundance compared with wt animals (FIG. 4h). In contrast, cardiac-specific knockdown of CaMKII-δ9 via transgenic expression of an shRNA targeting exon 16 of CaMKII-δ (which was performed according to the methods as described in Section 1.11 of GENERAL METHODS AND MATERIALS) markedly attenuated the TAC-induced cardiac hypertrophy, contractile dysfunction, and premature death in mice (FIG. 4i-k and FIG. 14f-i). TAC-induced cardiac DNA damage and cardiomyocyte death were also reduced by knockdown of CaMKII-δ9 (FIG. 4l, m). Concomitantly, UBE2T protein abundance was augmented in the CaMKII-δ9-deficient mouse heart (FIG. 14j). Although cardiac-specific overexpression of UBE2T per se displayed no grossly discernible phenotypes (FIG. 5), crossing the mice with CaMKII-δ9 tg mice effectively ameliorated the CaMKII-δ9-induced cardiac injury and dysfunction and markedly increased animal survival (FIG. 5). Thus, CaMKII-δ9-induced downregulation of UBE2T is a key mechanism underlying multiple insulting stimuli-induced cardiac DNA damage, cell death, and cardiomyopathy, leading to heart failure and animal death.

To further compare and contrast CaMKII-δ9 and CaMKII-δ2 functions and signaling mechanisms in vivo, the inventors constructed transgenic mice with cardiac-specific overexpression of CaMKII-δ2 at ˜8-fold over that of the wt animals, as was the case for CaMKII-δ9 tg mice (FIG. 14k). The CaMKII-δ2 tg mice were generated according to the methods as described in Section 1.12 of GENERAL METHODS AND MATERIALS.

It was found that CaMKII-δ2 tg mice exhibited cardiomyocyte death, cardiac hypertrophy and dysfunction, and animal death; but judged by all parameters, the detrimental effects were much less severe than those of CaMKII-δ9 tg animals (FIG. 14l-o). Moreover, the inventors detected neither a reduction in UBE2T abundance nor any increase in DNA damage in CaMKII-δ2 tg hearts (FIG. 14p, q). These results indicate that the two cytosolic CaMKII splice variants activate distinct signaling pathways and play different roles in cardiac physiology and pathology.

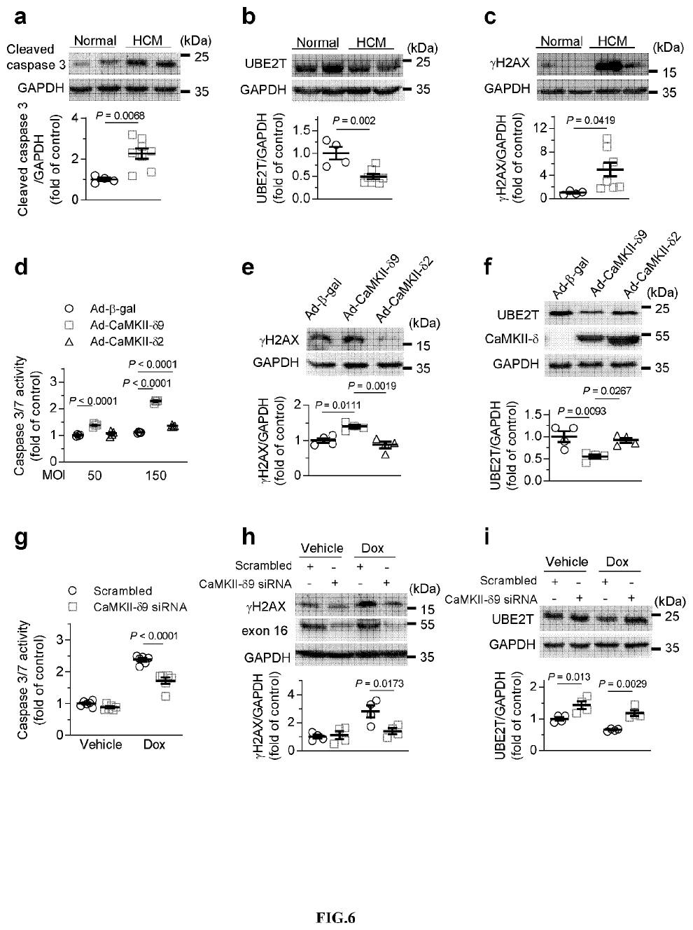

In myocardium from HCM patients, elevated CaMKII-δ9 (FIG. 1h), along with decreased UBE2T abundance and increased DNA damage (indexed by γH2AX), was accompanied by increased cardiomyocyte apoptosis (indexed by cleaved caspase 3) (FIG. 6a-c). Furthermore, in cardiomyocytes derived from human embryonic stem cells (which were obtained according to the methods as described in Section 1.26 of GENERAL METHODS AND MATERIALS), overexpression of CaMKII-δ9 led to more profound cell death than overexpression of CaMKII-δ2 (FIG. 6d). Concomitantly, CaMKII-δ9, but not 62, triggered UBE2T degradation and subsequent DNA damage (FIG. 6e, f). In contrast, CaMKII-δ9 knockdown effectively ameliorated Dox-induced UBE2T degradation, DNA damage, and cell death in these human cells (FIG. 6g-i).

### Example 6. CaMKII-δ9 Phosphorylates UBE2T at Ser110 and Promotes its Degradation

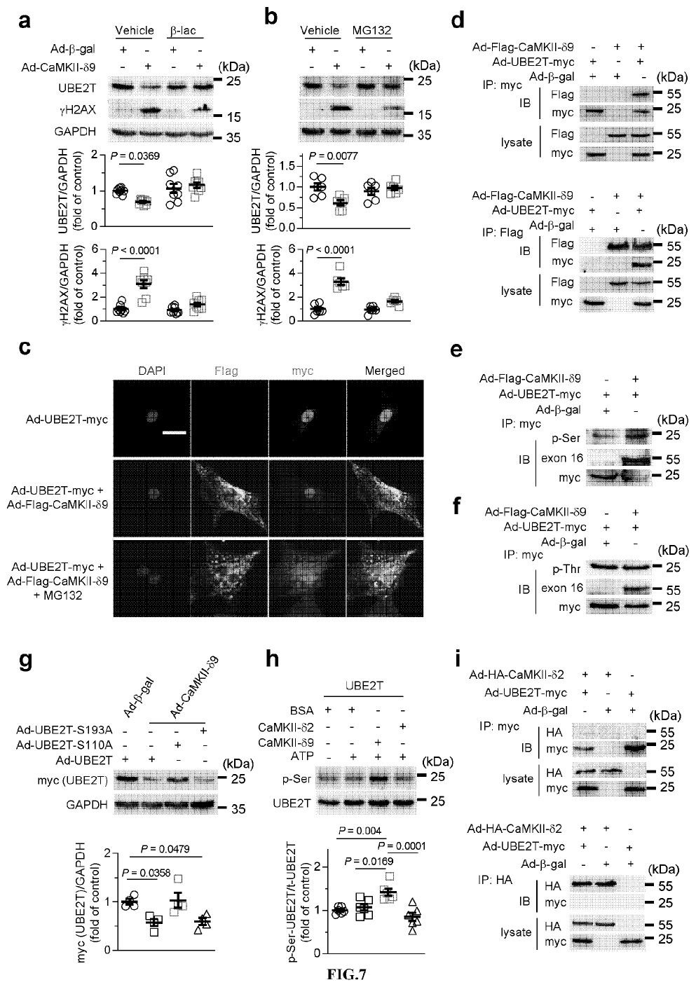

Since the UBE2T mRNA level was increased by CaMKII-δ9 overexpression, the downregulation of UBE2T at the protein level may be mediated by enhanced protein degradation. To test this hypothesis, the inventors used the proteasome inhibitors β-lac and MG132, and found that both fully abolished the CaMKII-δ9-induced reduction of the UBE2T protein level (FIG. 7a, b). UBE2T was primarily located in the nuclei of cardiomyocytes (FIG. 7c). In cells overexpressing CaMKII-δ9, UBE2T was still enriched in the nuclei, but its abundance was reduced (FIG. 7c). Notably, the proteasome inhibitor MG132 enabled UBE2T to be present in both the nuclear and cytosolic compartments (FIG. 7c). These results suggest that UBE2T is distributed in both the cytoplasm and nuclei of cardiomyocytes, and that its apparent enrichment in the nucleus is a consequence of CaMKII-δ9-mediated degradation of UBET2 in the cytoplasm. The CaMKII-δ9-induced increase of the UBE2T mRNA level is likely caused by cellular compensation for the reduction in its protein abundance.

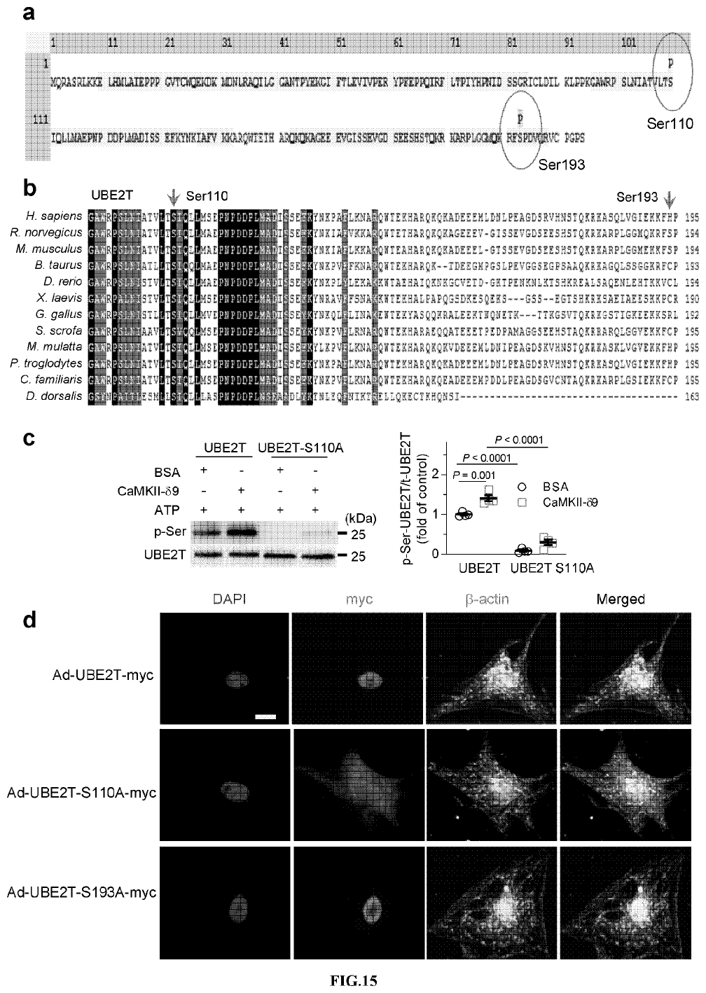

The inventors examined the potential physical interaction between CaMKII-δ9 and UBE2T in cardiomyocytes. Co-immunoprecipitation assays revealed that CaMKII-δ9 and UBE2T formed a protein complex (FIG. 7d). Overexpression of CaMKII-δ9 in NRVMs specifically elevated the serine phosphorylation of UBE2T (FIG. 7e), but not the threonine phosphorylation (FIG. 7f). To map out the phosphorylation site(s) of UBE2T for CaMKII-δ9, the inventors transfected CaMKII-δ9-free HEK 293 cells (human embryonic kidney cells) with myc-tagged UBE2T and CaMKII-δ9 plasmids. Cell lysates were immunoprecipitated with myc antibody and then subjected to MS analysis. The MS analysis was performed according to the methods as described in Section 1.8 of GENERAL METHODS AND MATERIALS. Two serine sites (Ser110 and Ser193) of UBE2T were identified as potential targets of CaMKII-δ9 (FIG. 15a). The inventors found that the UBE2T-S110A but not the UBE2T-S193A mutant was resistant to CaMKII-δ9-mediated degradation (FIG. 7g), indicating that phosphorylation of UBE2T at Ser110, a highly conserved site in multiple species (FIG. 15b), is essential for CaMKII-δ9-mediated UBE2T degradation.

Next, the inventors determined whether UBE2T is a direct substrate of CaMKII-δ9 with a cell-free kinase assay (which was performed according to the methods as described in Section 1.25 of GENERAL METHODS AND MATERIALS) using recombinant UBE2T protein in the presence or absence of CaMKII-δ9. The presence of recombinant CaMKII-δ9 protein significantly augmented the serine phosphorylation level of wt UBE2T, but not its S110A mutant (FIG. 7h and FIG. 15c). This result provided direct evidence that CaMKII-δ9 mediates UBE2T phosphorylation at Ser110. In addition, disrupting phosphorylation at S110 (UBE2T-S110A), but not at S193 (UBE2T-S193A), allowed UBE2T to reside in the cytoplasm as well as the nuclei (FIG. 15d), confirming that CaMKII-δ9 phosphorylates UBE2T at Ser110 and promotes proteasome-dependent UBE2T degradation in the cytoplasm, leading to an enrichment of UBE2T in the nuclei. Taken together, the in vivo and in vitro data show that upon cardiac injury, CaMKII-δ9 is activated and upregulated, which enhances the Ser110 phosphorylation and subsequent degradation of UBE2T, ultimately leading to myocardial DNA damage, genome instability, and cell death.

### Example 7. UBE2T is not Regulated by CaMKII-δ1, δ2 or δ3

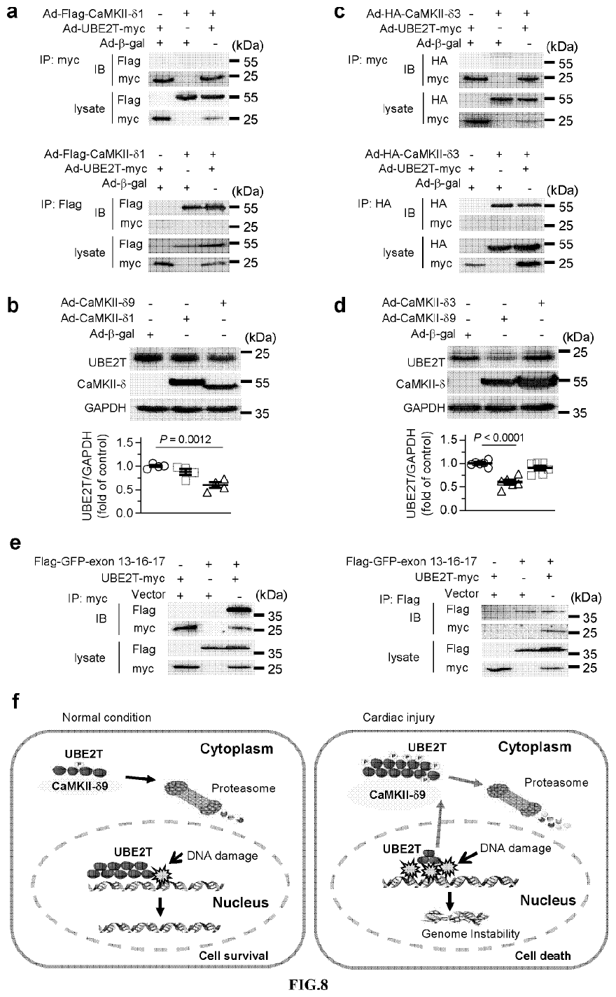

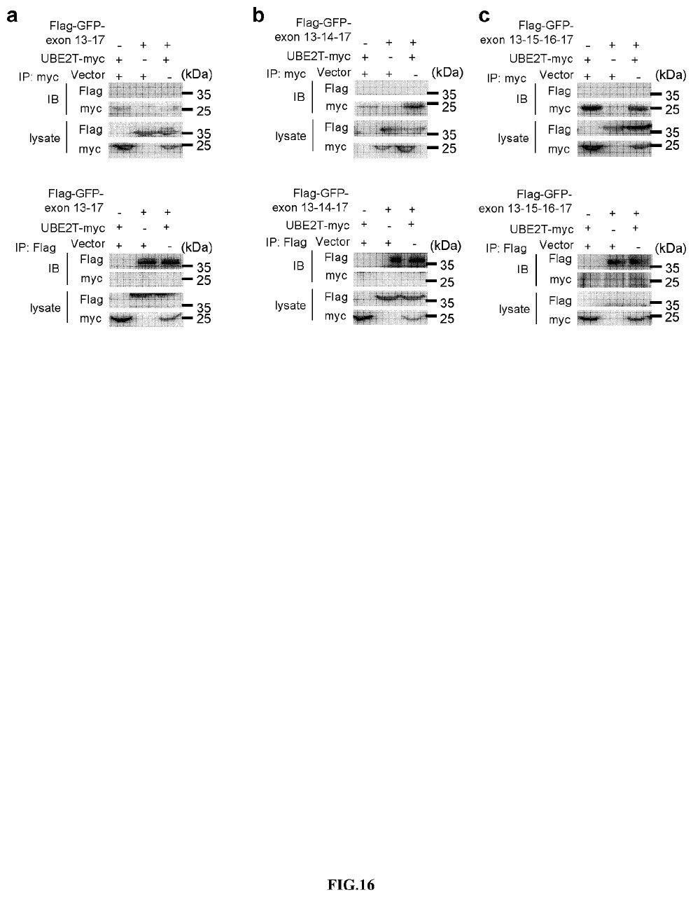

CaMKII-δ2, the minor cytosolic CaMKII-δ splice variant, neither interacted with UBE2T (FIG. 7i) nor increased its serine phosphorylation (FIG. 7h), implying that UBE2T is a specific substrate of CaMKII-δ9, but not 62. Furthermore, neither CaMKII-δ1 nor 63 interacted with UBE2T or induced its degradation (FIG. 8a-d), reaffirming that UBE2T is a selective target for CaMKII-δ9. To pinpoint the molecular basis of the splice variant-specific regulation of UBE2T by CaMKII-δ9, the inventors constructed plasmids expressing the peptides encoded by exons 13-15-16-17, 13-17, 13-14-17, and 13-16-17, the feature sequences of CaMKII-δ1, 62, 63, and 69, respectively (FIG. 9a). The plasmids were constructed according to the methods as described in Section 1.17 of GENERAL METHODS AND MATERIALS. The peptide encoded by exons 13-16-17, but not others, interacted with UBE2T (FIG. 8e and FIG. 16), suggesting that the feature sequence of CaMKII-δ9 (exons 13-16-17) is responsible for its substrate selectivity.

What claimed is:

1. A method of treating or preventing a CaMKII-mediated disease in a subject, comprising administering to the subject an effective amount of an antagonist of CaMKII-δ9.

2. A method of alleviating cardiac injury in a subject, comprising administering to the subject an effective amount of an antagonist of CaMKII-δ9.

3. A method of stimulating the level or activity of ubiquitin-conjugating enzyme in a subject, comprising administering to the subject an effective amount of an antagonist of CaMKII-δ9.

4. A method of preventing degradation of ubiquitin-conjugating enzyme in a subject, comprising administering to the subject an effective amount of an antagonist of CaMKII-δ9.

5. A method of preventing cardiomyocyte death in a sample, comprising contacting the sample with an effective amount of an antagonist of CaMKII-δ9.

6. A method of reducing DNA damage in a cell, comprising contacting the cell with an effective amount of an antagonist of CaMKII-δ9.

7. The method of any one of claims 1-6, wherein the antagonist is an antagonist for inhibiting the phosphorylation of ubiquitin-conjugating enzyme.

8. The method of claim 7, wherein the ubiquitin-conjugating enzyme is ubiquitin-conjugating enzyme 2T.

9. The method of claim 8, wherein the antagonist is an antagonist for inhibiting the phosphorylation of ubiquitin-conjugating enzyme 2T at Ser110.

10. The method of any one of claims 1-6, wherein the antagonist is a specific antagonist of CaMKII-δ9.

11. The method of any one of claims 1-6, wherein the antagonist inhibits the level or activity of CaMKII-δ9 but does not significantly inhibit the level or activity of CaMKII-δ2 or CaMKII-δ3.

12. The method of any one of claims 1-6, wherein the antagonist is an antibody that specifically recognizes CaMKII-δ9, a small molecule compound that binds to CaMKII-δ9, an RNAi molecule that targets an encoding sequence of CaMKII-δ9, an antisense nucleotide that targets an encoding sequence of CaMKII-δ9, or an agent that competes with CaMKII-δ9 to bind to its substrate.

13. The method of claim 12, wherein the antibody is a monoclonal antibody or a polyclonal antibody.

14. The method of claim 12, wherein the antibody is a humanized antibody, a chimeric antibody or a fully human antibody.

15. The method of any one of claims 12-14, wherein the antibody binds to the amino acid sequence encoded by exon 16 of CaMKII-5 gene.

16. The method of claim 12, wherein the RNAi molecule is a small interfering RNA (siRNA), a small hairpin RNA (shRNA) or a microRNA (miRNA).

17. The method of claim 16, wherein the RNAi molecule has 10-100 bases.

18. The method of claim 12, wherein the antisense nucleotide is modified to improve its stability.

19. The method of claim 12, wherein the RNAi molecule and the antisense nucleotide bind to exon 16 of CaMKII-δ gene.

20. The method of claim 19, wherein the RNAi molecule and the antisense nucleotide binds to exon 13 and exon 16 of CaMKII-δ gene, or exon 16 and exon 17 of CaMKII-δ gene, or exon 13 and exon 16 and exon 17 of CaMKII-δ gene.

21. The method of claim 12, wherein the agent that competes with CaMKII-δ9 to bind to its substrate is a vector that expresses CaMKII-δ9 which is without phosphorylation or oxidation function.

22. The method of claim 21, wherein the vector is an adeno-associated virus (AAV), an adenovirus, a lentivirus, a retrovirus, or a plasmid.

23. The method of claim 22, wherein the AAV is AAV1, AAV2, AAV5, AAV8, AAV9 or AAVrh10.

24. The method of any one of claims 1-4, wherein the subject is a human or non-human primate.

25. The method of claim 24, wherein the non-human primate is a rhesus monkey.

26. The method of claim 1, wherein the CaMKII-mediated disease is associated with an increased level or activity of CaMKII-δ9.

27. The method of claim 1, wherein the CaMKII-mediated disease is a heart disease or a metabolic disease.

28. The method of claim 27, wherein the heart disease is selected from the group consisting of cardiomyopathy, myocarditis, diabetic heart disease, myocardial ischemia, cardiac ischemia/reperfusion injury, myocardial infarction, heart failure, arrhythmia, heart rupture, angina, cardiac hypertrophy, cardiac injury, hypertensive heart disease, rheumatic heart disease, angina, myocarditis, coronary heart disease and pericarditis.

29. The method of claim 27, wherein the metabolic disease is selected from the group consisting of insulin resistance, obesity, diabetes, hypertension, dyslipidemia, diabetic cerebrovascular diseases, diabetic ocular complications, diabetic neuropathy, diabetic foot, hyperinsulinemia, hypercholesterolemia, hyperglycaemia, hyperlipemia, gout and hyperuricemia.

30. A method for diagnosing a CaMKII-mediated disease in a subject comprising:
a) obtaining a test biological sample of the subject,
b) detecting a level or activity of CaMKII-δ9 in the test biological sample; wherein the level or activity of CaMKII-δ9 detected in the test biological sample of the subject is indicative of the subject developing or with an increased probability of developing a CaMKII-mediated disease.

31. The method of claim 30, wherein the level or activity of CaMKII-δ9 in the test biological sample is detected by contacting the sample with a reagent that specifically binds to CaMKII-δ9.

32. The method of claim 30, wherein the level or activity of CaMKII-δ9 detected in the test biological sample is compared to a reference level or activity of CaMKII-δ9 detected in a reference sample.

33. The method of claim 32, wherein a higher level or activity of the CaMKII-δ9 detected in the test biological sample than the reference level or activity of CaMKII-δ9 is indicative of the subject developing or with an increased probability of developing a CaMKII-mediated disease.

34. The method of claim 32, wherein the reference sample is from a healthy subject or is a sample obtained from the same subject earlier or later than the test biological sample.

35. The method of any one of claims 30-34, wherein the test biological sample is from the heart of the subject.

36. The method of claim 35, wherein the subject is a human or non-human primate.

37. A kit for diagnosing a CaMKII-mediated disease in a subject, comprising an antibody or an antibody fragment that specifically recognizes CaMKII-δ9.

38. A method for identifying a molecule which inhibits the activity of CaMKII-δ9, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

39. A method for identifying a molecule which inhibits the phosphorylation of CaMKII-δ9, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

40. A method for identifying a molecule which treats or prevents a CaMKII-mediated disease, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

41. A method for identifying a molecule which alleviates cardiac injury, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

42. A method for identifying a molecule which prevents cardiomyocyte death, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

43. A method for identifying a molecule which reduces DNA damage, comprising contacting the molecule with CaMKII-δ9 and ubiquitin-conjugating enzyme 2T, and determining whether the phosphorylation of ubiquitin-conjugating enzyme 2T is inhibited, wherein the inhibition of the phosphorylation of ubiquitin-conjugating enzyme 2T identifies a molecule that inhibits CaMKII-δ9.

44. The method of any one of claims 38-43, wherein the phosphorylation of ubiquitin-conjugating enzyme 2T is at Ser110.

45. A biomarker for diagnosing a CaMKII-mediated disease in a subject, wherein the biomarker includes the full length protein sequence of CaMKII-δ9 or a fragment thereof.

46. The biomarker of claim 45, wherein the biomarker includes the amino acid sequence set forth in SEQ ID NOs: 1-5.

47. Use of CaMKII-δ9 as a biomarker for diagnosing a CaMKII-mediated disease in a subject.


SEQUENCE LISTING (98 sequences)

SEQ ID NO: 1 - protein, 14 aa, Artificial Sequence
  the amino acid sequence of exon 16 of CaMKII- delta gene
EPQTTVIHNPDGNK

SEQ ID NO: 2 - protein, 27 aa, Artificial Sequence
  the amino acid sequence of exons 13-16 of CaMKII-delta gene
AAKSLLKKPDGVKEPQTTVIHNPDGNK

SEQ ID NO: 3 - protein, 30 aa, Artificial Sequence
  the amino acid sequence of exons 16-17 of CaMKII-delta gene
EPQTTVIHNPDGNKESTESSNTTIEDEDVK

SEQ ID NO: 4 - protein, 43 aa, Artificial Sequence
  the amino acid sequence of exons 13-16-17 of CaMKII-delta gene
AAKSLLKKPDGVKEPQTTVIHNPDGNKESTESSNTTIEDEDVK

SEQ ID NO: 5 - protein, 513 aa, Homo sapiens
MASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKEPQTTVIHNPDGNKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIKPPCIPNGKENFSGGTSLWQNI

SEQ ID NO: 6 - DNA, 42 bp, Artificial Sequence
  the nucleic acid sequence of human and rat exon 16 of CaMKII-delta gene
gagccccaaactactgtaatccacaaccctgatggaaacaag

SEQ ID NO: 7 - DNA, 42 bp, Artificial Sequence
  the nucleic acid sequence of mouse exon 16 of CaMKII-delta gene
gagccccaaactactgtaatccacaaccctgacggaaacaag

SEQ ID NO: 8 - DNA, 81 bp, Artificial Sequence
  the nucleic acid sequence of human exons 13-16 of CaMKII-delta gene
gcagccaagagtttgttgaagaaaccagatggagtaaaggagccccaaactactgtaatccacaaccctgatggaaacaag

SEQ ID NO: 9 - DNA, 81 bp, Artificial Sequence
  the nucleic acid sequence of rat exons 13-16 of CaMKII-delta gene
gcagccaagagtttgttgaagaaaccggatggggtaaaggagccccaaactactgtaatccacaaccctgatggaaacaag

SEQ ID NO: 10 - DNA, 81 bp, Artificial Sequence
  the nucleic acid sequence of mouse exons 13-16 of CaMKII-delta gene
gcagccaagagtttattgaagaaaccagatggggtaaaggagccccaaactactgtaatccacaaccctgacggaaacaag

SEQ ID NO: 11 - DNA, 90 bp, Artificial Sequence
  the nucleic acid sequence of human exons 16-17 of CaMKII-delta gene
gagccccaaactactgtaatccacaaccctgatggaaacaaggagtcaactgagagttcaaatacaacaattgaggatgaagatgtgaaa

SEQ ID NO: 12 - DNA, 90 bp, Artificial Sequence
  the nucleic acid sequence of rat exons 16-17 of CaMKII-delta gene
gagccccaaactactgtaatccacaaccctgatggaaacaaggagtcaactgagagctcaaataccaccattgaggatgaagacgtgaaa

SEQ ID NO: 13 - DNA, 90 bp, Artificial Sequence
  the nucleic acid sequence of mouse exons 16-17 of CaMKII-delta gene
gagccccaaactactgtaatccacaaccctgacggaaacaaggagtcaactgagagctcaaacaccaccattgaggatgaagacgtgaaa

SEQ ID NO: 14 - DNA, 129 bp, Artificial Sequence
  the nucleic acid sequence of human exons 13-16-17 of CaMKII-delta gene
gcagccaagagtttgttgaagaaaccagatggagtaaaggagccccaaactactgtaatccacaaccctgatggaaacaaggagtcaactgagagttcaaatacaacaattgaggatgaagatgtgaaa

SEQ ID NO: 15 - DNA, 129 bp, Artificial Sequence
  the nucleic acid sequence of rat exons 13-16-17 of CaMKII-delta gene
gcagccaagagtttgttgaagaaaccggatggggtaaaggagccccaaactactgtaatccacaaccctgatggaaacaaggagtcaactgagagctcaaataccaccattgaggatgaagacgtgaaa

SEQ ID NO: 16 - DNA, 129 bp, Artificial Sequence
  the nucleic acid sequence of mouse exons 13-16-17 of CaMKII-delta gene
gcagccaagagtttattgaagaaaccagatggggtaaaggagccccaaactactgtaatccacaaccctgacggaaacaaggagtcaactgagagctcaaacaccaccattgaggatgaagacgtgaaa

SEQ ID NO: 17 - DNA, 1542 bp, Homo sapiens
atggcttcgaccacaacctgcaccaggttcacggacgagtatcagcttttcgaggagcttggaaagggggcattctcagtggtgagaagatgtatgaaaattcctactggacaagaatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaaggagccccaaactactgtaatccacaaccctgatggaaacaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatctaa

SEQ ID NO: 18 - DNA, 1542 bp, Rattus rattus
atggcttcgaccaccacctgcacccggttcaccgacgagtatcagctcttcgaggagctcggaaagggggcattctcagtggtgagaagatgcatgaaaatccctactggacaagagtatgctgccaaaattatcaacaccaaaaagctttctgctagggatcatcagaaactggaaagggaagctagaatctgccgtctcttgaagcaccccaatattgtgagacttcatgacagcatatccgaagagggcttccattacttggtgtttgacttagttactggtggcgaactctttgaagacatagtggcaagagaatattacagtgaggctgatgccagtcattgtatacaacagattctagagagtgtaaatcattgtcacctaaatggcatagttcacagggacctgaagcctgagaatttgcttttagctagcaaatccaaaggagcagctgtgaaactggcagacttcggcttagccatagaagttcaaggcgaccagcaggcgtggtttggttttgctggcacacctgggtatctttctccagaagtcctacgtaaagatccttatggaaaaccagtggacatgtgggcatgtggcgtcatcctctacatcttgctggtgggatacccacccttctgggatgaagatcagcatagactgtatcagcagatcaaggctggagcgtacgattttccatcaccagaatgggacacagtgacacctgaagccaaagacctcatcaacaaaatgctgaccatcaaccctgccaaacgcatcacagcctctgaggccctgaaacacccatggatctgtcaacgttctactgttgcctccatgatgcacaggcaggagactgtagactgcttgaagaaatttaatgctcgacggaaattgaagggtgccatcttgacaactatgctggctacgagaaatttttcagcagccaagagtttgttgaagaaaccggatggggtaaaggagccccaaactactgtaatccacaaccctgatggaaacaaggagtcaactgagagctcaaataccaccattgaggatgaagacgtgaaagcacgaaagcaagagatcatcaaagtcactgagcagctgattgaagctatcaacaatggggacttcgaggcttacacgaaaatctgtgatccaggcctcactgcctttgaacccgaagcattgggcaacttagtggaagggatggactttcacagattctactttgaaaatgctttgcccaaaatcaataaaccaatccacactatcatcctgaaccctcacgtacacctggtaggggatgatgcagcctgcatagcatacattcggctcacacagtacatggatggaaatggaatgccaaagacaatgcagtcagaagagactcgagtgtggcaccgccgtgatgggaagtggcagaatattcactttcatcgttcggggtccccaacagtccccatcaagccaccctgtattccaaatgggaaagaaaacttctcaggaggcacctctttgtggcaaaacatctga

SEQ ID NO: 19 - DNA, 1542 bp, Mus musculus
atggcttcgaccaccacctgcacccggttcaccgacgagtatcagctctttgaggagctcggaaagggggcgttctcagtggtgagaagatgtatgaaaatccctactggacaagagtatgctgccaaaattatcaacaccaaaaagctttctgctagggaccatcagaaactggaaagggaagctagaatctgccgtctcttgaagcaccccaatattgtgagacttcacgacagtatatcggaggagggcttccattacttggtgtttgacttagtgactggtggcgaactgtttgaagacatagtggcaagagaatattacagtgaagctgatgccagtcattgtatacaacagattctagagagtgtaaatcattgtcacctaaatggcatagttcacagggacctgaagcctgagaatttgcttttagctagcaagtccaaaggagcagctgtgaagctggcagacttcggcttagccatagaagttcaaggcgaccagcaggcatggtttggttttgctggcacacctgggtatctttctccagaagtcctgcgtaaagatccttatggaaaaccagtggatatgtgggcatgcggtgtcatcctctacatcttgctggtgggatacccacccttctgggatgaagatcagcatagactgtatcagcagatcaaggccggagcttacgattttccgtcaccagaatgggatacagtgacacctgaagccaaagacctcatcaacaaaatgctgaccatcaaccctgccaaacgtatcacagcctctgaggccctgaaacacccatggatctgtcaacgctctactgttgcctccatgatgcacaggcaggagactgtagactgcttgaagaaatttaatgctagacggaaactgaagggcgccatcttgacaactatgctggctacgagaaatttttcagcagccaagagtttattgaagaaaccagatggggtaaaggagccccaaactactgtaatccacaaccctgacggaaacaaggagtcaactgagagctcaaacaccaccattgaggatgaagacgtgaaagcacgaaaacaggagatcatcaaagtcactgagcaactgattgaagctatcaacaatggggactttgaggcttacacaaaaatctgtgatccaggcctcactgcctttgaacctgaagcattgggcaacttagtggaagggatggactttcacagattctactttgaaaatgctttgtccaaaagcaataaaccaatccacacgatcatcctcaacccacacgttcacctggtaggggatgacgcagcctgcatcgcatacattcggctcacacagtacatggacggaagcgggatgccaaagaccatgcagtcagaagagacgcgcgtgtggcaccgccgtgatgggaagtggcagaatgttcactttcaccgttcggggtcccccacagtacccatcaagccaccctgtattccaaatgggaaagagaacttctcaggaggcacctctttgtggcaaaacatctga

SEQ ID NO: 20 - DNA, 21 bp, Artificial Sequence
  forward primer_human
gccagggcacagcccggaccg

SEQ ID NO: 21 - DNA, 21 bp, Artificial Sequence
  reverse primer_human
gagaatgcagaagtggcactg

SEQ ID NO: 22 - DNA, 20 bp, Artificial Sequence
  forward primer_rhesus monkey
gggcacagcccggaccaagg

SEQ ID NO: 23 - DNA, 20 bp, Artificial Sequence
  reverse primer_rhesus monkey
atgcagaagtggcactgttg

SEQ ID NO: 24 - DNA, 20 bp, Artificial Sequence
  forward primer_rat
gggcacagcccggaccaagg

SEQ ID NO: 25 - DNA, 20 bp, Artificial Sequence
  reverse primer_rat
atgcagaagtggcactgttg

SEQ ID NO: 26 - DNA, 20 bp, Artificial Sequence
  forward primer_mouse
ggcgagctactttcggacac

SEQ ID NO: 27 - DNA, 20 bp, Artificial Sequence
  reverse primer_mouse
gaagaagtggcactgttgac

SEQ ID NO: 28 - protein, 18 aa, Artificial Sequence
  antigenic epitope_exon 21
PPCIPNGKENFSGGTSLW

SEQ ID NO: 29 - protein, 15 aa, Artificial Sequence
  antigenic epitope_exon 16
CEPQTTVIHNPDGNK

SEQ ID NO: 30 - protein, 17 aa, Artificial Sequence
  proteotypic trypsin peptide_e20-21
  MISC_FEATURE (17)..(17): this amino acid is labeled with heavy isotope
SGSPTVPIKPPCIPNGK

SEQ ID NO: 31 - protein, 9 aa, Artificial Sequence
  proteotypic trypsin peptide_e20-22
  MISC_FEATURE (9)..(9): this amino acid is labled with heavy isotope
SGSPTVPIK

SEQ ID NO: 32 - protein, 22 aa, Artificial Sequence
  proteotypic trypsin peptide_e13-17
  MISC_FEATURE (22)..(22): this amino acid is labeled with heavy isotope
KPDGVKESTESSNTTIEDEDVK

SEQ ID NO: 33 - protein, 11 aa, Artificial Sequence
  proteotypic trypsin peptide_e13-14
  MISC_FEATURE (11)..(11): this amino acid is labeled with heavy isotope
SLLKKPDGVKK

SEQ ID NO: 34 - protein, 20 aa, Artificial Sequence
  proteotypic trypsin peptide_e13-16
  MISC_FEATURE (20)..(20): this amino acid is labeled with heavy isotope
KPDGVKEPQTTVIHNPDGNK

SEQ ID NO: 35 - DNA, 24 bp, Artificial Sequence
  forward primer
gtatcgataagcttgccaccatgg

SEQ ID NO: 36 - DNA, 22 bp, Artificial Sequence
  reverse primer
catgaagtcgcacaatattagg

SEQ ID NO: 37 - DNA, 106 bp, Artificial Sequence
  miR30-based CaMKII-delta 9 shRNA
gaaggtatattgctgttgacagtgagcgcaatccacaaccctgacggaaatagtgaagccacagatgtatttccgtcagggttgtggattatgcctactgcctcgg

SEQ ID NO: 38 - DNA, 21 bp, Artificial Sequence
  forward primer
cttcaccgagggcctatttcc

SEQ ID NO: 39 - DNA, 20 bp, Artificial Sequence
  reverse primer
ccgtaggtggcatcgccctc

SEQ ID NO: 40 - DNA, 20 bp, Artificial Sequence
  forward primer
cggtatcgataagcttggcc

SEQ ID NO: 41 - DNA, 20 bp, Artificial Sequence
  reverse primer
tcacaatattggggtgcttc

SEQ ID NO: 42 - DNA, 20 bp, Artificial Sequence
  forward primer
atagaagcctagcccacacc

SEQ ID NO: 43 - DNA, 20 bp, Artificial Sequence
  reverse primer
gatctgtggtggctcaaatg

SEQ ID NO: 44 - DNA, 20 bp, Artificial Sequence
  forward primer_18S
ggaagggcaccaccaggagt

SEQ ID NO: 45 - DNA, 20 bp, Artificial Sequence
  reverse primer_18S
tgcagccccggacatctaag

SEQ ID NO: 46 - DNA, 21 bp, Artificial Sequence
  forward primer_a-MHC
atcattcccaacgagcgaaag

SEQ ID NO: 47 - DNA, 21 bp, Artificial Sequence
  reverse primer_a-MHC
aagtccccatagagaatgcgg

SEQ ID NO: 48 - DNA, 21 bp, Artificial Sequence
  forward primer_ANP
ttcttcctcgtcttggccttt

SEQ ID NO: 49 - DNA, 23 bp, Artificial Sequence
  reverse primer_ANP
gacctcatcttctaccggcatct

SEQ ID NO: 50 - DNA, 18 bp, Artificial Sequence
  forward primer_ATF3
catcaacaacagacctct

SEQ ID NO: 51 - DNA, 18 bp, Artificial Sequence
  reverse primer_ATF3
cattcacactctccagtt

SEQ ID NO: 52 - DNA, 18 bp, Artificial Sequence
  forward primer_Bdkrb1
ccttctacgctctgttaa

SEQ ID NO: 53 - DNA, 19 bp, Artificial Sequence
  reverse primer_Bdkrb1
ccaggatgtgatagttgaa

SEQ ID NO: 54 - DNA, 18 bp, Artificial Sequence
  forward primer_b-MHC
atgtgccggaccttggaa

SEQ ID NO: 55 - DNA, 22 bp, Artificial Sequence
  reverse primer_b-MHC
cctcgggttagctgagagatca

SEQ ID NO: 56 - DNA, 21 bp, Artificial Sequence
  forward primer_BNP
aagtcctagccagtctccaga

SEQ ID NO: 57 - DNA, 21 bp, Artificial Sequence
  reverse primer_BNP
gagctgtctctgggccatttc

SEQ ID NO: 58 - DNA, 31 bp, Artificial Sequence
  forward primer_CaMKII-d2
ccagatggggtaaaggagtcaactgagagct

SEQ ID NO: 59 - DNA, 31 bp, Artificial Sequence
  reverse primer_CaMKII-d2/3/9
tcagatgttttgccacaaagaggtgcctcct

SEQ ID NO: 60 - DNA, 32 bp, Artificial Sequence
  forward primer_CaMKII-d3
aaaaggaagtccagttcgagtgttcagatgat

SEQ ID NO: 61 - DNA, 25 bp, Artificial Sequence
  forward primer_CaMKII-d9
gtaaaggagccccaaactactgtaa

SEQ ID NO: 62 - DNA, 18 bp, Artificial Sequence
  forward primer_Cdo1
gacctcatccgaatcttg

SEQ ID NO: 63 - DNA, 18 bp, Artificial Sequence
  reverse primer_Cdo1
cagcacagaatcatcaga

SEQ ID NO: 64 - DNA, 18 bp, Artificial Sequence
  forward primer_Chac1
gctacgacactaaggaag

SEQ ID NO: 65 - DNA, 18 bp, Artificial Sequence
  reverse primer_Chac1
cgcaacaagtattcaagg

SEQ ID NO: 66 - DNA, 21 bp, Artificial Sequence
  forward primer_COX-2
ggcacaaatatgatgttcgca

SEQ ID NO: 67 - DNA, 22 bp, Artificial Sequence
  reverse primer_COX-2
cctcgcttctgatctgtcttga

SEQ ID NO: 68 - DNA, 19 bp, Artificial Sequence
  forward primer_CSF2
ctatacaagcagggtctac

SEQ ID NO: 69 - DNA, 20 bp, Artificial Sequence
  reverse primer_CSF2
ctatttcacagtcagtttcc

SEQ ID NO: 70 - DNA, 19 bp, Artificial Sequence
  forward primer_Ccna2
gaaggtctcaggttatcag

SEQ ID NO: 71 - DNA, 18 bp, Artificial Sequence
  reverse primer_Ccna2
gtctggtcattctgtctc

SEQ ID NO: 72 - DNA, 18 bp, Artificial Sequence
  forward primer_Ereg
agagaaggatggagactt

SEQ ID NO: 73 - DNA, 19 bp, Artificial Sequence
  reverse primer_Ereg
gctgataactgcttgtaga

SEQ ID NO: 74 - DNA, 18 bp, Artificial Sequence
  forward primer_Has1
aactggctgctaactatg

SEQ ID NO: 75 - DNA, 18 bp, Artificial Sequence
  reverse primer_Has1
agtctccttacacctacc

SEQ ID NO: 76 - DNA, 18 bp, Artificial Sequence
  forward primer_Hpgd
caggagtgaacaatgaga

SEQ ID NO: 77 - DNA, 18 bp, Artificial Sequence
  reverse primer_Hpgd
ataaggttagcagccatc

SEQ ID NO: 78 - DNA, 18 bp, Artificial Sequence
  forward primer_LIF
gatttcccacctttccat

SEQ ID NO: 79 - DNA, 18 bp, Artificial Sequence
  reverse primer_LIF
ctgtagtcgcattgagtt

SEQ ID NO: 80 - DNA, 18 bp, Artificial Sequence
  forward primer_PAI-2
aatccattcagccttctc

SEQ ID NO: 81 - DNA, 20 bp, Artificial Sequence
  reverse primer_PAI-2
cagcgttgtatgtattcttc

SEQ ID NO: 82 - DNA, 18 bp, Artificial Sequence
  forward primer_Plk1
tattacctgcctcaccat

SEQ ID NO: 83 - DNA, 19 bp, Artificial Sequence
  reverse primer_Plk1
cttgtccgaatagtctacc

SEQ ID NO: 84 - DNA, 20 bp, Artificial Sequence
  forward primer_Reg3b
aatatacctggattggactc

SEQ ID NO: 85 - DNA, 20 bp, Artificial Sequence
  reverse primer_Reg3b
caatgtggactgtagataga

SEQ ID NO: 86 - DNA, 19 bp, Artificial Sequence
  forward primer_UBE2T
agccaatacaccttatgag

SEQ ID NO: 87 - DNA, 20 bp, Artificial Sequence
  reverse primer_UBE2T
tccttccactagaatcaatg

SEQ ID NO: 88 - RNA, 19 nt, Artificial Sequence
  the seqeunce of siRNA_Scrambled
ucccaauccuagggacaaa

SEQ ID NO: 89 - RNA, 19 nt, Artificial Sequence
  the sequence of siRNA_CaMKII-d9
uccacaacccugauggaaa

SEQ ID NO: 90 - RNA, 19 nt, Artificial Sequence
  COX-2 siRNA1
ccuuccuucggaauucaau

SEQ ID NO: 91 - RNA, 19 nt, Artificial Sequence
  COX-2 siRNA2
ccaugggugugaaaggaaa

SEQ ID NO: 92 - RNA, 19 nt, Artificial Sequence
  COX-2 siRNA3
ccaguaucagaaccgcauu

SEQ ID NO: 93 - RNA, 19 nt, Artificial Sequence
  UBE2T siRNA1
ccacuguauugaccucuau

SEQ ID NO: 94 - RNA, 19 nt, Artificial Sequence
  UBE2T siRNA2
ccaugcagcgauucuuuaa

SEQ ID NO: 95 - RNA, 19 nt, Artificial Sequence
  FANCD2 siRNA1
gcggcugaacauaaggcuu

SEQ ID NO: 96 - RNA, 19 nt, Artificial Sequence
  FANCD2 siRNA2
gcugucaucuguccgucua

SEQ ID NO: 97 - RNA, 19 nt, Artificial Sequence
  FANCI siRNA1
ccugagagccauccucaaa

SEQ ID NO: 98 - RNA, 19 nt, Artificial Sequence
  FANCI siRNA2
ccaucauccuuacugccuu